(19) United States
(12) Patent Application Publication
(10) Pub. No.: US 2022/0175758 A1
(43) Pub. Date: Jun. 9, 2022

(54) COMPOSITIONS AND METHODS TO TREAT NON-ALCOHOLIC FATTY LIVER DISEASES (NAFLD)

(71) Applicant: Coherus Biosciences, Inc., Redwood City, CA (US)

(72) Inventors: Christos Mantzoros, Watertown, MA (US); Glenn D. Rosen, Los Altos, CA (US)

(21) Appl. No.: 17/600,194

(22) PCT Filed: Dec. 27, 2019
(86) PCT No.: PCT/US2019/068688

§ 371 Date: Sep. 30, 2021

Related U.S. Application Data
(60) Provisional application No. 62/829,071, filed on Apr. 4, 2019.
(60) Provisional application No. 62/829,098, filed on Apr. 4, 2019.
(60) Provisional application No. 62/829,097, filed on Apr. 4, 2019.
(60) Provisional application No. 62/829,235, filed on Apr. 4, 2019.
(60) Provisional application No. 62/829,253, filed on Apr. 4, 2019.
(60) Provisional application No. 62/829,258, filed on Apr. 4, 2019.

(51) Int. Cl. A61K 31/473; A61K 31/437; A61K 31/4439; A61K 31/501; A61K 31/444; A61K 45/06; A61P 1/16

(52) U.S. Cl. CPC A61K 31/473; A61K 31/437; A61K 31/4439; A61P 1/16; A61K 31/444; A61K 45/06; A61K 31/501

(57) ABSTRACT

Provided herein are methods and combination therapies useful for the treatment of non-alcoholic fatty liver diseases (NAFLD). In particular, provided herein are methods and combination therapies for treating NAFLD by administering a combination therapy comprising (a) the compound of Formula (I), or a pharmaceutically acceptable salt thereof, and (b) an additional therapeutic agent, or a pharmaceutically acceptable salt thereof. Also provided are pharmaceutical compositions and pharmaceutical combinations comprising the compound of Formula (I), or a pharmaceutically acceptable salt thereof, and an additional therapeutic agent, or a pharmaceutically acceptable salt thereof.

### CROSS REFERENCE TO RELATED APPLICATIONS

This application claims priority to U.S. Provisional Application Ser. No. 62/829,071, filed on Apr. 4, 2019, 62/829,098, filed on Apr. 4, 2019, 62/829,097, filed on Apr. 4, 2019, 62/829,235, filed Apr. 4, 2019, 62/829,253, filed on Apr. 4, 2019, and 62/829,258, filed Apr. 4, 2019, each of which is herein incorporated by reference in its entirety.

### TECHNICAL FIELD

The present disclosure relates to methods and combination therapies useful for the treatment of non-alcoholic fatty liver diseases (NAFLD). In particular, this disclosure relates to methods and combination therapies for treating NAFLD by administering a combination therapy comprising a PPARγ inhibitor that is the compound of Formula (I), or a pharmaceutically acceptable salt thereof, and an additional therapeutic agent, or a pharmaceutically acceptable salt thereof.

### BACKGROUND

Non-alcoholic fatty liver disease (NAFLD) is characterized by the presence of hepatic fat accumulation in the absence of secondary causes of hepatic steatosis including excessive alcohol consumption, other known liver diseases, or long-term use of a steatogenic medication (Perumpail et al., World J Gastroenterol. 2017, 23(47):8263-8438 and Chalasani et al., Hepatology. 2018, 67(1):328-357). NAFLD encompasses two categories: simple non-alcoholic fatty liver (NAFL) and non-alcoholic steatohepatitis (NASH). Typically, NAFL has a more indolent course of progression whereas NASH is a more severe form associated with inflammation that may progress more rapidly to end-stage liver disease. NAFL and/or NASH may also include scarring of the liver known as liver fibrosis or in a more severe form, liver cirrhosis. Scarring of the liver reduces liver function up to and including liver failure.

NAFLD is currently the most common liver disease in the world (Perumpail et al., World J Gastroenterol. 2017, 23(47):8263-8438) with approximately one-fourth of the adult population suffering from NAFLD worldwide (Sumida, et al., J Gastroenterol. 2018, 53:362-376). There are many risk factors associated with NAFLD including hypertension, obesity, diabetes, and hyperipidemia with a particularly close association with type II diabetes mellitus and NAFLD (Vernon et al., Aliment Pharmacol Ther. 2011, 34:274-285).

Lifestyle interventions including dietary caloric restriction and exercise are the most effective methods of prevention and treatment for NAFLD (Sumida, et al., J Gastroenterol. 2018, 53:362-376). However, these can be difficult treatments to follow. Thus, there is a need for pharmaceuticals to treat NAFLD. Current pharmaceutical treatments that have been proposed or tested in prior trials, although are not yet approved for NAFLD include vitamin E, ω3 fatty acid, statin, metformin, orlistat, thiazolidinediones (“TZDs”), urodeoxycholic acid, pioglitazone, and pentoxifilline (Sumida, et al., J Gastroenterol. 2018, 53:362-376). However, there is currently no approved pharmacotherapy for NAFLD.

While some treatments have shown early promise in clinical trials, others have failed to shows efficacy as a monotherapy. For example, selonsertib—an apoptosis signal-regulating kinase 1 inhibitor—failed to meet the primary endpoint as a monotherapy in the STELLAR-4 phase 3 clinical trial. While a single treatment may not be efficacious in treating NAFLD, a combination of therapies may be efficacious. There is a need to identify combinations of therapeutic agents that will efficacious in treating NAFLD.

In subjects having NAFLD, oxidative stress activates apoptosis signal-regulating kinase 1 (ASK1), a serine/threonine signaling kinase, leading to phosphorylation of downstream targets (e.g., p38 and JNK), thus activating stress response pathways that worsen hepatic inflammation, apoptosis, and fibrosis. Budas, et al., J. Hepatol., 2016, 64(Suppl.): S170; and Wang, et al., Nat. Med., 2017, 23: 439-49. However, inhibitors of this pathway have failed to shows efficacy as a monotherapy. For example, selonsertib—an ASK1 inhibitor—failed to meet the primary endpoint in the STELLAR-4 phase 3 clinical trial.

Acetyl-CoA carboxylase (ACC) is a biotin-dependent enzyme that catalyzes the irreversible carboxylation of acetyl-CoA to produce malonyl-CoA, which is a substrate for the biosynthesis of fatty acids. Tong, L., Cell. Molec. Life Sci., 2005, 62(16): 1784-803. In mammals, two main isoforms of ACC are expressed, ACC1 and ACC2, which differ in both tissue distribution and function, though both isoforms are highly expressed in the liver where both fatty acid oxidation and synthesis are important. Barber, et al., Biochim. et Biophys. Acta., 2005, 1733(1): 1-28. Indeed, inhibition of liver-specific isoform ACC1 in mice reduced hepatic triglyceride levels by inhibiting fatty acid biosynthesis and activating fatty acid beta oxidation in the liver. Abu-Elheiga, et al., Proc. Nat. Acad. Sci. USA, 2005, 102: 12011-16.

The pathogenesis of NAFLD also includes disruption of several sophisticated signaling networks within both hepatocytes and parenchymal cells. Emerging evidence suggests that increased hepatocyte apoptosis contributes to liver inflammation and fibrogenesis in NAFLD. Brenner, et al., J. Hepatol., 2013, 59(3): 583-94. Indeed, apoptotic markers are increasingly recognized as indicators of NAFLD, even at sub-lethal expression levels. Feldstein et al., Hepatology, 2009, 50(4): 1072-78. Caspases are a family of cysteine-proteases that execute the final phase of apoptosis: caspases 2, 3, 7, 8, 9, and 10 are apoptotic caspases, and caspases 1, 4, 5, 11, and 12 are associated with inflammation. Shalini, et al., Cell Death Differ., 2015, 22(4): 526-39. Caspases are synthesized as inactive pro-caspases that are activated following an appropriate stimulus. Activation typically involves dimerization or oligomerization of pro-caspases, followed by cleavage into a two distinct subunits, which associated to form an active heterodimeric (or sometimes heterotetrameric) caspase. Shi, Y., Cell, 2004, 117(7): 855-58.

Mineralocorticoid receptor antagonists, initially developed for the treatment of edema related to heart and kidney disease, also modulate aldosterone- and glucocorticoid-induced adipocyte differentiation, with the link between the mechanisms controlling adipocyte differentiation and energy balance highlights the mineralocorticoid signaling system as a potential candidate for the development of obesity and its associated metabolic complications. Delyani, Kidney Int., 2000, 57(4): 1408-11 and Caprio, et al., Endocrinology, 2011, 152(1): 113-25. Local activation of the mineralocorticoid receptor in the liver is related to several hormones, including aldosterone and cortisol, which are often elevated in subjects with NAFLD. Tarantino and Finelli, W. J. Gastroenterol., 2013, 19: 6735-43. Dysregulation of mineralocorticoid receptor expression in adipose tissue has also been documented in these subjects. Wada, et al., Am. J. Physiol. Endocrinol. Metab., 2013, 305: E1415-25.

Dyslipidaemia and metabolic syndrome are also very common in patients with NAFLD and the metabolic syndrome and contribute to negative outcomes. Dyson, et al., Frontline Gastroenterol., 2014, 5: 277-86. One mechanism to address dyslipidemia is via administration of statins, which are competitive inhibitors of HMG-CoA reductase, the rate-limiting enzyme of the mevalonate pathway, which is critical for cholesterol biosynthesis. The lipid-lowering effect of statins may other effects, such as slowing progression of hepatic inflammation and fibrosis via anti-inflammatory, antiapoptotic, and/or antithrombotic, effects. Tziomalos, et al., Metabolism, 2015, 64: 1215-23.

Another mechanism to address dyslipidemia is via inhibition of proprotein convertase subtilisin/kexin type 9 (PCSK9). PCSK9 is a serine proprotein convertase enzyme that functions in the proteolytic processing and maturation of secretory proteins. Seidah, et al., Proc. Nat. Acad. Sci. USA, 2003, 100: 928-33. Mechanistic studies have demonstrated that overexpression or gain-of-function mutations in PCSK9 reduced low density lipoprotein receptor (LDLR) protein levels in liver, which significantly increased circulating plasma cholesterol. Horton et al., Trends Biochem. Sci., 2007, 32: 71-77. Deletion of Pcsk9 in mice (or knockdown with antisense oligonucleotides) resulted in increased LDLR levels, accelerated the clearance of low density lipoprotein cholesterol (LDLc), and reduced circulating cholesterol levels. Graham, et al., J. Lipid Res., 2007, 48: 763-67; and Cohen, et al., Nat. Genet., 2005, 37: 161-65.

While a single treatment may not be efficacious in treating NAFLD, a combination of therapies may be efficacious. There is a need to identify combinations of therapeutic agents that will efficacious in treating NAFLD.

### SUMMARY

Provided herein in some embodiments is a method of treating non-alcoholic fatty liver disease (NAFLD) in a subject in need thereof comprising administering to the subject
(a) the compound of Formula (I),

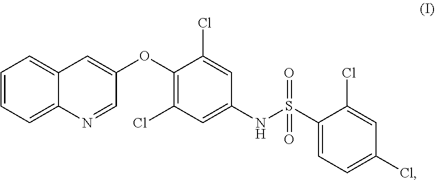 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ASK1 inhibitor, or a pharmaceutically acceptable salt thereof, wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(a) the compound of Formula (I),

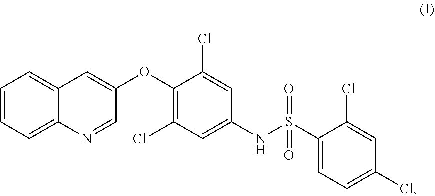 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ASK1 inhibitor, or a pharmaceutically acceptable salt thereof, to the selected subject, wherein the amounts of (a) and (b) together are effective in treating NAFLD.

Provided herein in some embodiments is a method of treating a subject, the method comprising:

identifying a subject having non-alcoholic fatty liver disease (NAFLD); and

administering
(a) the compound of Formula (I),

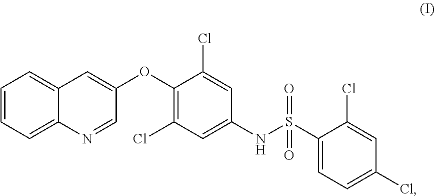 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ASK1 inhibitor, or a pharmaceutically acceptable salt thereof, to the selected subject, wherein the amounts of (a) and (b) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

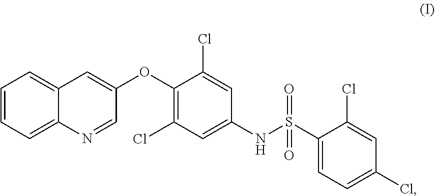 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an ASK1 inhibitor, or a pharmaceutically acceptable salt thereof.

Provided herein in some embodiments is a method of treating a subject, the method comprising:

selecting a subject having non-alcoholic fatty liver disease (NAFLD); and

administering
(a) a therapeutically effective amount of the compound of Formula (I),

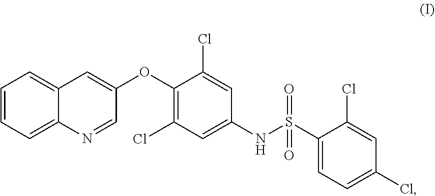 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an ASK1 inhibitor, or a pharmaceutically acceptable salt thereof, to the selected subject.

Provided herein in some embodiments is a method of treating fibrosis in a subject in need thereof comprising administering to the subject
(a) the compound of Formula (I),

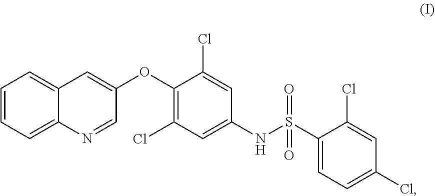 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ASK1 inhibitor, or a pharmaceutically acceptable salt thereof, wherein the amounts of (a) and (b) together are effective in treating fibrosis.

Provided herein in some embodiments is a method of treating fibrosis in a subject in need thereof comprising administering to the subject
(a) a therapeutically effective amount of the compound of Formula (I),

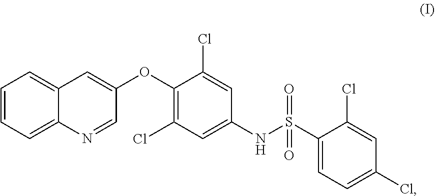 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an ASK1 inhibitor, or a pharmaceutically acceptable salt thereof.

Provided herein in some embodiments is a method of treating hepatic steatosis in a subject in need thereof comprising administering to the subject
(a) the compound of Formula (I),

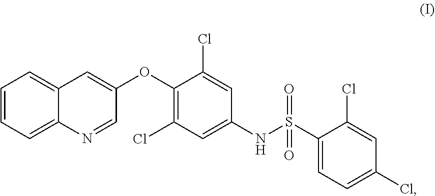 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ASK1 inhibitor, or a pharmaceutically acceptable salt thereof,

wherein the amounts of (a) and (b) together are effective in treating hepatic steatosis.

Provided herein in some embodiments is a method of treating hepatic steatosis in a subject in need thereof comprising administering to the subject
(a) a therapeutically effective amount of the compound of Formula (I),

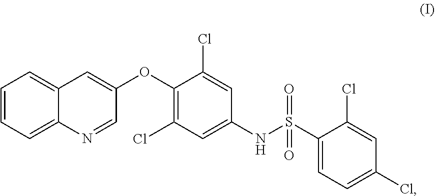 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an ASK1 inhibitor, or a pharmaceutically acceptable salt thereof.

In some more particular embodiments, (a) and (b) are administered concurrently.

In some more particular embodiments, (a) and (b) are administered sequentially in either order.

Provided herein in some embodiments is a method of treating non-alcoholic fatty liver disease (NAFLD) in a subject in need thereof comprising administering to the subject
(a) the compound of Formula (I),

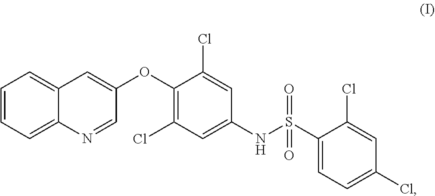 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ACC inhibitor, or a pharmaceutically acceptable salt thereof,

wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(a) the compound of Formula (I),

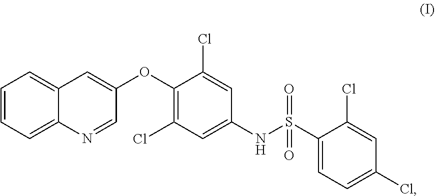 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ACC inhibitor, or a pharmaceutically acceptable salt thereof, to the selected subject,

wherein the amounts of (a) and (b) together are effective in treating NAFLD.

Provided herein in some embodiments is a method of treating a subject, the method comprising:
identifying a subject having non-alcoholic fatty liver disease (NAFLD); and

administering
(a) the compound of Formula (I),

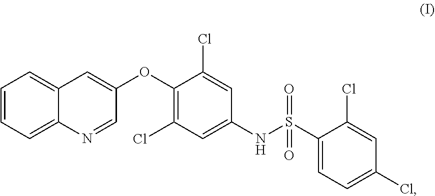 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an ACC inhibitor, or a pharmaceutically acceptable salt thereof, to the selected subject,

wherein the amounts of (a) and (b) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

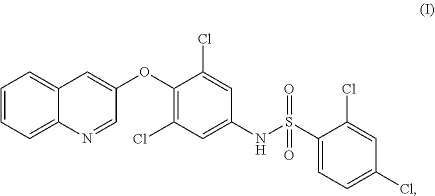 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an ACC inhibitor, or a pharmaceutically acceptable salt thereof.

Provided herein in some embodiments is a method of treating a subject, the method comprising:

selecting a subject having non-alcoholic fatty liver disease (NAFLD); and

administering
(a) a therapeutically effective amount of the compound of Formula (I),

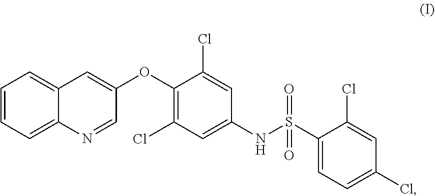 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an ACC inhibitor, or a pharmaceutically acceptable salt thereof, to the selected subject.

Provided herein in some embodiments is a method of treating fibrosis in a subject in need thereof comprising administering to the subject
(c) the compound of Formula (I),

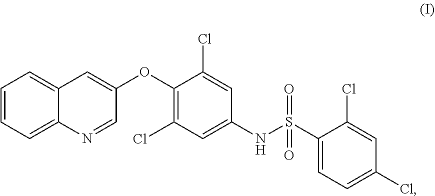 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) an ACC inhibitor, or a pharmaceutically acceptable salt thereof,

wherein the amounts of (a) and (b) together are effective in treating fibrosis.

Provided herein in some embodiments is a method of treating fibrosis in a subject in need thereof comprising administering to the subject
(c) a therapeutically effective amount of the compound of Formula (I),

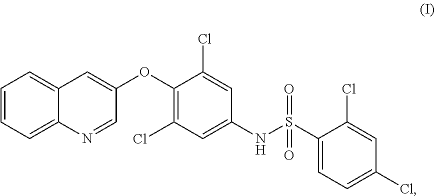 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of an ACC inhibitor, or a pharmaceutically acceptable salt thereof.

Provided herein in some embodiments is a method of treating hepatic steatosis in a subject in need thereof comprising administering to the subject
(c) the compound of Formula (I),

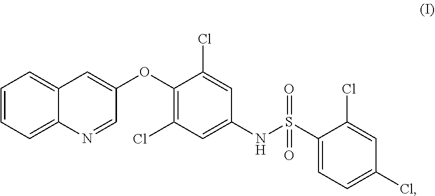 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) an ACC inhibitor, or a pharmaceutically acceptable salt thereof,

wherein the amounts of (a) and (b) together are effective in treating hepatic steatosis.

Provided herein in some embodiments is a method of treating hepatic steatosis in a subject in need thereof comprising administering to the subject
(c) a therapeutically effective amount of the compound of Formula (I),

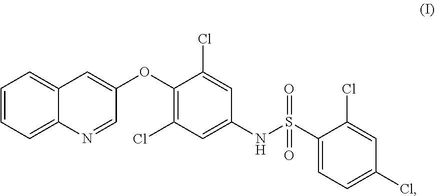 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of an ACC inhibitor, or a pharmaceutically acceptable salt thereof.

Provided herein in some embodiments is a method of treating non-alcoholic fatty liver disease (NAFLD) in a subject in need thereof comprising administering to the subject
(c) the compound of Formula (I),

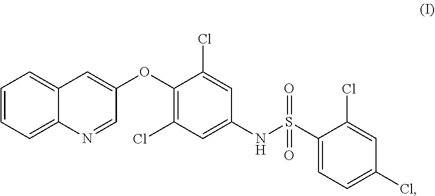 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a caspase inhibitor, or a pharmaceutically acceptable salt thereof,

wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(c) the compound of Formula (I),

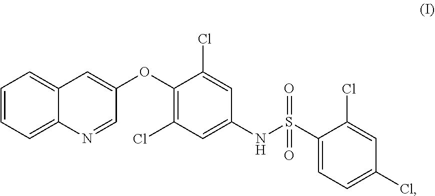 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a caspase inhibitor, or a pharmaceutically acceptable salt thereof, to the selected subject,

wherein the amounts of (a) and (b) together are effective in treating NAFLD.

Provided herein in some embodiments is a method of treating a subject, the method comprising:
identifying a subject having non-alcoholic fatty liver disease (NAFLD); and

administering
(c) the compound of Formula (I),

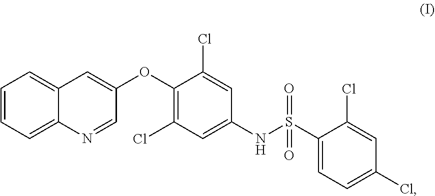 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a caspase inhibitor, or a pharmaceutically acceptable salt thereof, to the selected subject,

wherein the amounts of (a) and (b) together are effective in treating NAFLD.

Provided herein in some embodiments is a method of treating non-alcoholic fatty liver disease (NAFLD) in a subject in need thereof comprising administering to the subject
(c) a therapeutically effective amount of the compound of Formula (I),

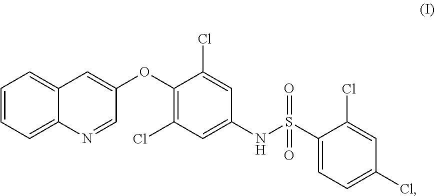 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of a caspase inhibitor, or a pharmaceutically acceptable salt thereof.

Provided herein in some embodiments is a method of treating a subject, the method comprising:

selecting a subject having non-alcoholic fatty liver disease (NAFLD); and

administering
(c) a therapeutically effective amount of the compound of Formula (I),

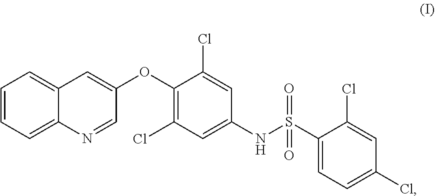 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of a caspase inhibitor, or a pharmaceutically acceptable salt thereof, to the selected subject.

Provided herein in some embodiments is a method of treating fibrosis in a subject in need thereof comprising administering to the subject
(e) the compound of Formula (I),

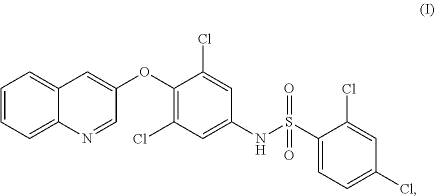 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a caspase inhibitor, or a pharmaceutically acceptable salt thereof,

wherein the amounts of (a) and (b) together are effective in treating fibrosis.

Provided herein in some embodiments is a method of treating fibrosis in a subject in need thereof comprising administering to the subject
(e) a therapeutically effective amount of the compound of Formula (I),

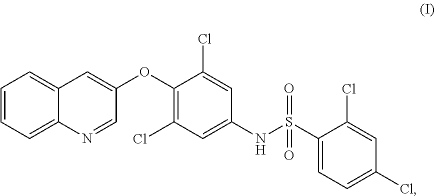 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a therapeutically effective amount of a caspase inhibitor, or a pharmaceutically acceptable salt thereof.

Provided herein in some embodiments is a method of treating hepatic steatosis in a subject in need thereof comprising administering to the subject
(e) the compound of Formula (I),

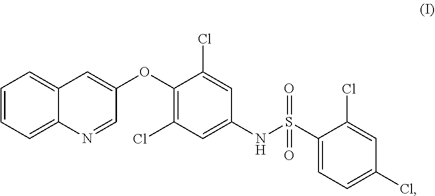 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a caspase inhibitor, or a pharmaceutically acceptable salt thereof,

wherein the amounts of (a) and (b) together are effective in treating hepatic steatosis.

Provided herein in some embodiments is a method of treating hepatic steatosis in a subject in need thereof comprising administering to the subject
(e) a therapeutically effective amount of the compound of Formula (I),

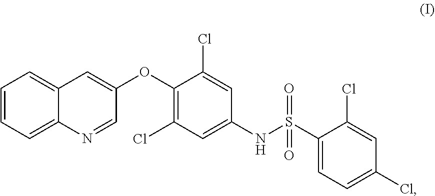 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and

a therapeutically effective amount of a caspase inhibitor, or a pharmaceutically acceptable salt thereof.

Provided herein in some embodiments is a method of treating non-alcoholic fatty liver disease (NAFLD) in a subject in need thereof comprising administering to the subject
(a) the compound of Formula (I),

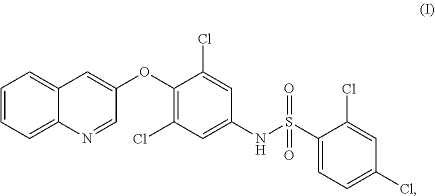 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an MCR antagonist, or a pharmaceutically acceptable salt thereof,

wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(a) the compound of Formula (I),

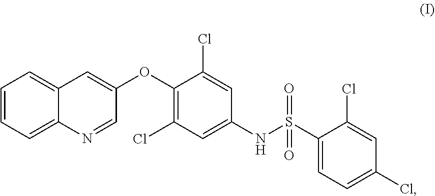 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an MCR antagonist, or a pharmaceutically acceptable salt thereof, to the selected subject,

wherein the amounts of (a) and (b) together are effective in treating NAFLD.

Provided herein in some embodiments is a method of treating a subject, the method comprising:

identifying a subject having non-alcoholic fatty liver disease (NAFLD); and

administering
(a) the compound of Formula (I),

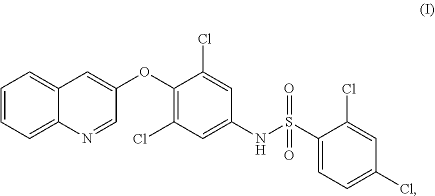 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an MCR antagonist, or a pharmaceutically acceptable salt thereof, to the selected subject,

wherein the amounts of (a) and (b) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

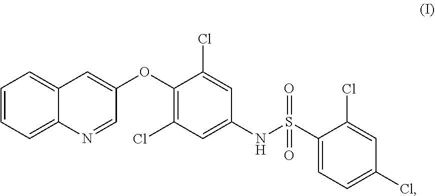 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an MCR antagonist, or a pharmaceutically acceptable salt thereof.

Provided herein in some embodiments is a method of treating a subject, the method comprising:

selecting a subject having non-alcoholic fatty liver disease (NAFLD); and

administering
(a) a therapeutically effective amount of the compound of Formula (I),

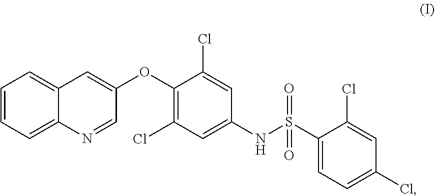 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an MCR antagonist, or a pharmaceutically acceptable salt thereof, to the selected subject.

Provided herein in some embodiments is a method of treating fibrosis in a subject in need thereof comprising administering to the subject
(a) the compound of Formula (I),

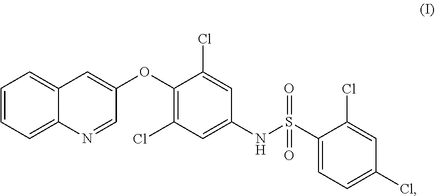 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an MCR antagonist, or a pharmaceutically acceptable salt thereof,

wherein the amounts of (a) and (b) together are effective in treating fibrosis.

Provided herein in some embodiments is a method of treating fibrosis in a subject in need thereof comprising administering to the subject
(a) a therapeutically effective amount of the compound of Formula (I),

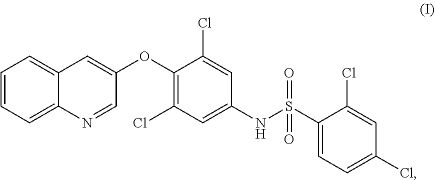 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an MCR antagonist, or a pharmaceutically acceptable salt thereof.

Provided herein in some embodiments is a method of treating hepatic steatosis in a subject in need thereof comprising administering to the subject
(a) the compound of Formula (I),

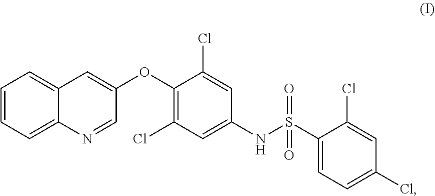 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an MCR antagonist, or a pharmaceutically acceptable salt thereof,

wherein the amounts of (a) and (b) together are effective in treating hepatic steatosis.

Provided herein in some embodiments is a method of treating hepatic steatosis in a subject in need thereof comprising administering to the subject
(a) a therapeutically effective amount of the compound of Formula (I),

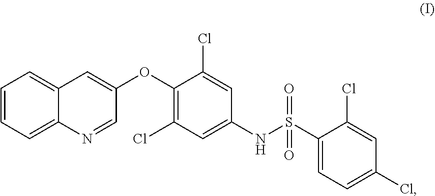 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an MCR antagonist, or a pharmaceutically acceptable salt thereof.

Provided herein in some embodiments is a method of treating non-alcoholic fatty liver disease (NAFLD) in a subject in need thereof comprising administering to the subject
(c) the compound of Formula (I),

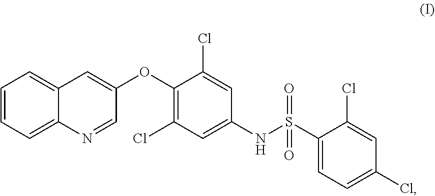 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a statin, or a pharmaceutically acceptable salt thereof,

wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(c) the compound of Formula (I),

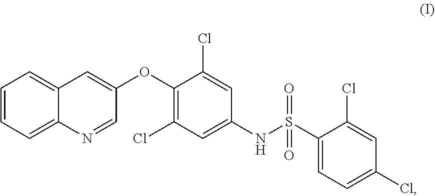 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a statin, or a pharmaceutically acceptable salt thereof, to the selected subject,

wherein the amounts of (a) and (b) together are effective in treating NAFLD.

Provided herein in some embodiments is a method of treating a subject, the method comprising:
identifying a subject having non-alcoholic fatty liver disease (NAFLD); and

administering
(c) the compound of Formula (I),

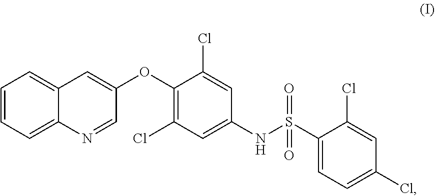 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a statin, or a pharmaceutically acceptable salt thereof, to the selected subject,

wherein the amounts of (a) and (b) together are effective in treating NAFLD.

Provided herein in some embodiments is a method of treating non-alcoholic fatty liver disease (NAFLD) in a subject in need thereof comprising administering to the subject
(c) a therapeutically effective amount of the compound of Formula (I),

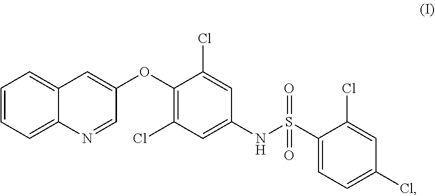 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of a statin, or a pharmaceutically acceptable salt thereof.

Provided herein in some embodiments is a method of treating a subject, the method comprising:

selecting a subject having non-alcoholic fatty liver disease (NAFLD); and

administering
(c) a therapeutically effective amount of the compound of Formula (I),

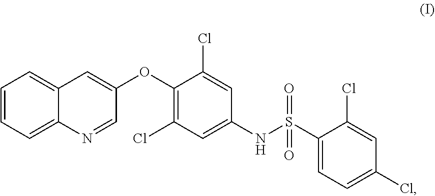 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of a statin, or a pharmaceutically acceptable salt thereof, to the selected subject.

Provided herein in some embodiments is a method of treating fibrosis in a subject in need thereof comprising administering to the subject
(c) the compound of Formula (I),

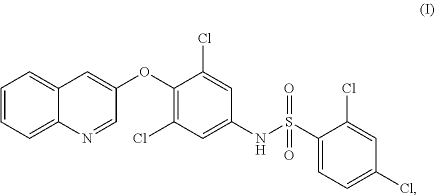 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a statin, or a pharmaceutically acceptable salt thereof,

wherein the amounts of (a) and (b) together are effective in treating fibrosis.

Provided herein in some embodiments is a method of treating fibrosis in a subject in need thereof comprising administering to the subject
(c) a therapeutically effective amount of the compound of Formula (I),

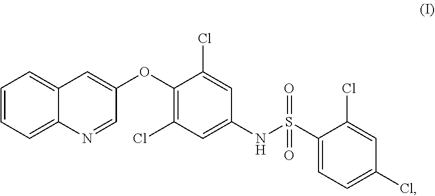 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of a statin, or a pharmaceutically acceptable salt thereof.

Provided herein in some embodiments is a method of treating hepatic steatosis in a subject in need thereof comprising administering to the subject
(c) the compound of Formula (I),

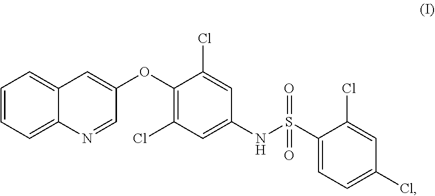 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a statin, or a pharmaceutically acceptable salt thereof,

wherein the amounts of (a) and (b) together are effective in treating hepatic steatosis.

Provided herein in some embodiments is a method of treating hepatic steatosis in a subject in need thereof comprising administering to the subject
(c) a therapeutically effective amount of the compound of Formula (I),

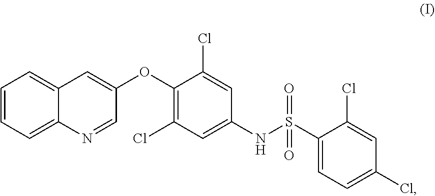 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(d) a therapeutically effective amount of a statin, or a pharmaceutically acceptable salt thereof.

Provided herein in some embodiments is a method of treating non-alcoholic fatty liver disease (NAFLD) in a subject in need thereof comprising administering to the subject
(e) the compound of Formula (I),

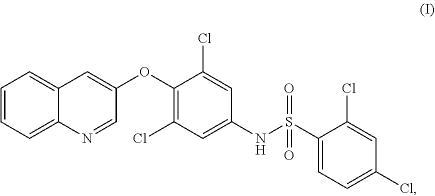 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a PCSK9 inhibitor,

wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(e) the compound of Formula (I),

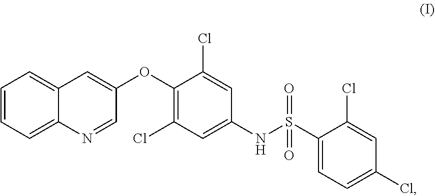 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a PCSK9 inhibitor, to the selected subject,

wherein the amounts of (a) and (b) together are effective in treating NAFLD.

Provided herein in some embodiments is a method of treating a subject, the method comprising:
identifying a subject having non-alcoholic fatty liver disease (NAFLD); and

administering
(e) the compound of Formula (I),

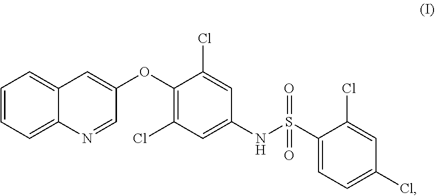 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a PCSK9 inhibitor, to the selected subject,

wherein the amounts of (a) and (b) together are effective in treating NAFLD.

Provided herein in some embodiments is a method of treating non-alcoholic fatty liver disease (NAFLD) in a subject in need thereof comprising administering to the subject
(e) a therapeutically effective amount of the compound of Formula (I),

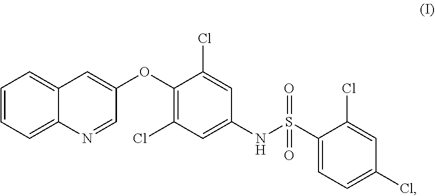 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a therapeutically effective amount of a PCSK9 inhibitor.

Provided herein in some embodiments is a method of treating a subject, the method comprising:

selecting a subject having non-alcoholic fatty liver disease (NAFLD); and

administering
(e) a therapeutically effective amount of the compound of Formula (I),

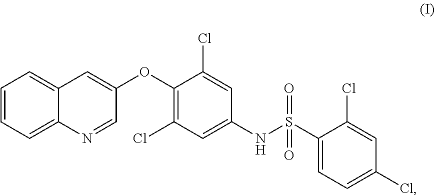 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a therapeutically effective amount of a PCSK9 inhibitor, to the selected subject.

Provided herein in some embodiments is a method of treating fibrosis in a subject in need thereof comprising administering to the subject
(e) the compound of Formula (I),

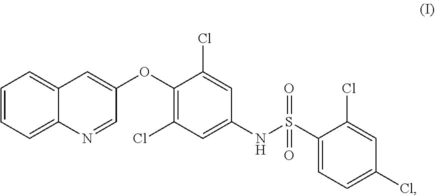 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a PCSK9 inhibitor,

wherein the amounts of (a) and (b) together are effective in treating fibrosis.

Provided herein in some embodiments is a method of treating fibrosis in a subject in need thereof comprising administering to the subject
(e) a therapeutically effective amount of the compound of Formula (I),

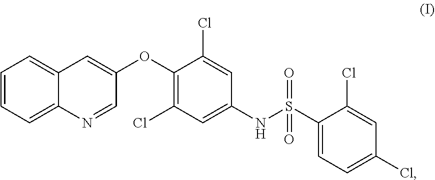 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a therapeutically effective amount of a PCSK9 inhibitor.

Provided herein in some embodiments is a method of treating hepatic steatosis in a subject in need thereof comprising administering to the subject
(e) the compound of Formula (I),

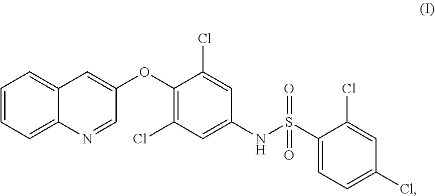 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a PCSK9 inhibitor,

wherein the amounts of (a) and (b) together are effective in treating hepatic steatosis.

Provided herein in some embodiments is a method of treating hepatic steatosis in a subject in need thereof comprising administering to the subject
(e) a therapeutically effective amount of the compound of Formula (I),

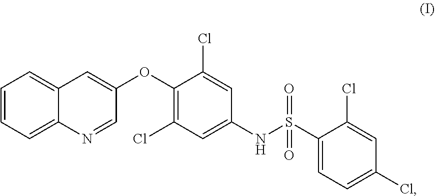 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(f) a therapeutically effective amount of a PCSK9 inhibitor.

Provided herein in some embodiments is a pharmaceutical composition comprising
(a) the compound of Formula (I),

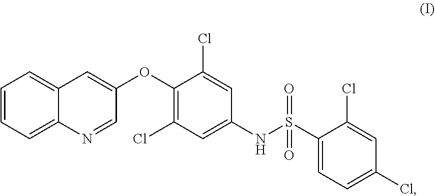 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an ASK1 inhibitor, or a pharmaceutically acceptable salt thereof, and one or more pharmaceutical excipients.

Provided herein in some embodiments is a pharmaceutical combination comprising
(a) the compound of Formula (I),

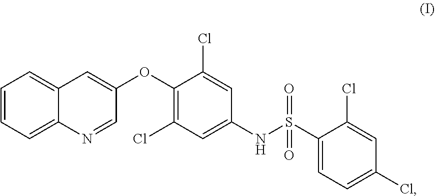 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an ASK1 inhibitor, or a pharmaceutically acceptable salt thereof, and

one or more pharmaceutical excipients, for concurrent or sequential administration during a period of time for use in the treatment of non-alcoholic fatty liver disease (NAFLD).

Provided herein in some embodiments is a pharmaceutical composition comprising
(c) the compound of Formula (I),

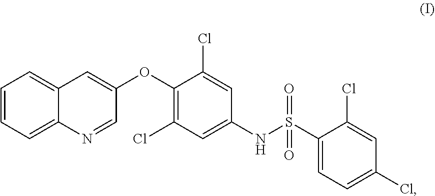 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(d) an ACC inhibitor, or a pharmaceutically acceptable salt thereof, and one or more pharmaceutical excipients.

In some embodiments, the amounts of (a) and (b) together are effective in treating NAFLD.

In some embodiments, the amount of (a) is a therapeutically effective amount and the amount of (b) is a therapeutically effective amount.

Provided herein in some embodiments is a pharmaceutical combination comprising
(c) the compound of Formula (I),

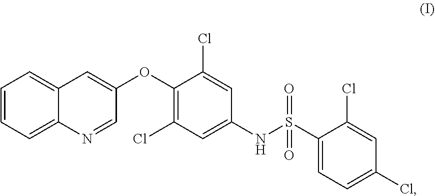 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(d) an ACC inhibitor, or a pharmaceutically acceptable salt thereof, and

one or more pharmaceutical excipients, for concurrent or sequential administration during a period of time for use in the treatment of non-alcoholic fatty liver disease (NAFLD).

Provided herein in some embodiments is a pharmaceutical composition comprising
(e) the compound of Formula (I),

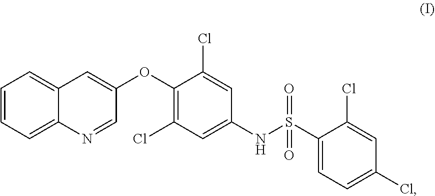 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(f) a caspase inhibitor, or a pharmaceutically acceptable salt thereof, and one or more pharmaceutical excipients.

Provided herein in some embodiments is a pharmaceutical combination comprising
(e) the compound of Formula (I),

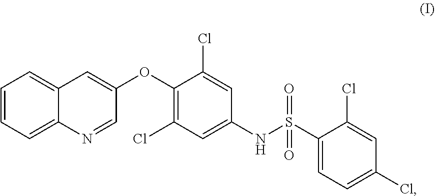 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(f) a caspase inhibitor, or a pharmaceutically acceptable salt thereof, and

one or more pharmaceutical excipients, for concurrent or sequential administration during a period of time for use in the treatment of non-alcoholic fatty liver disease (NAFLD).

Provided herein in some embodiments is a pharmaceutical composition comprising
(a) the compound of Formula (I),

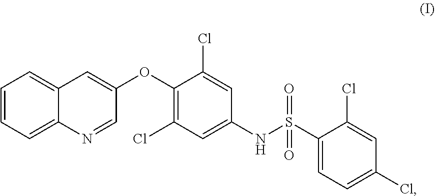 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an MCR antagonist, or a pharmaceutically acceptable salt thereof, and one or more pharmaceutical excipients.

Provided herein in some embodiments is a pharmaceutical combination comprising
(a) the compound of Formula (I),

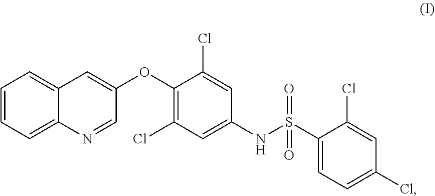 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an MCR antagonist, or a pharmaceutically acceptable salt thereof, and

one or more pharmaceutical excipients, for concurrent or sequential administration during a period of time for use in the treatment of non-alcoholic fatty liver disease (NAFLD).

Provided herein in some embodiments is a pharmaceutical composition comprising
(c) the compound of Formula (I),

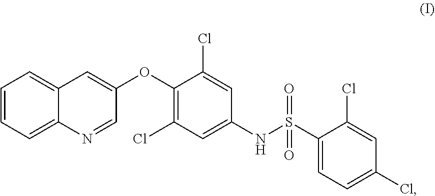 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(d) a statin, or a pharmaceutically acceptable salt thereof, and one or more pharmaceutical excipients.

In some embodiments, the amounts of (a) and (b) together are effective in treating NAFLD.

In some embodiments, the amount of (a) is a therapeutically effective amount and the amount of (b) is a therapeutically effective amount.

Provided herein in some embodiments is a pharmaceutical combination comprising
(c) the compound of Formula (I),

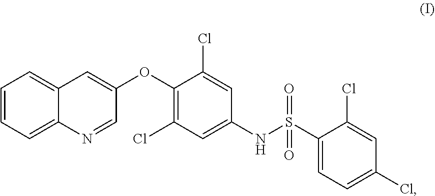 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(d) a statin, or a pharmaceutically acceptable salt thereof, and

one or more pharmaceutical excipients, for concurrent or sequential administration during a period of time for use in the treatment of non-alcoholic fatty liver disease (NAFLD).

Provided herein in some embodiments is a pharmaceutical composition comprising
(e) the compound of Formula (I),

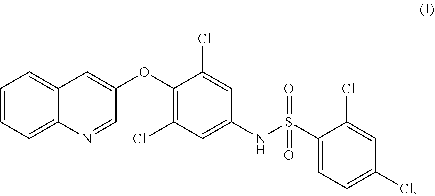 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(f) a PCSK9 inhibitor, and

one or more pharmaceutical excipients.

In some embodiments, the amounts of (a) and (b) together are effective in treating NAFLD.

In some embodiments, the amount of (a) is a therapeutically effective amount and the amount of (b) is a therapeutically effective amount.

Provided herein in some embodiments is a pharmaceutical combination comprising
(e) the compound of Formula (I),

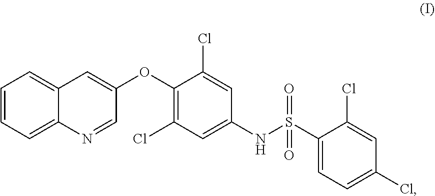 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(f) a PCSK9 inhibitor, and

one or more pharmaceutical excipients, for concurrent or sequential administration during a period of time for use in the treatment of non-alcoholic fatty liver disease (NAFLD).

In some embodiments, the amounts of (a) and (b) together are effective in treating NAFLD.

In some embodiments of the pharmaceutical compositions provided herein, the pharmaceutical compositions comprise at least one pharmaceutically acceptable carrier.

In some more particular embodiments, a method as provided herein comprises administering a pharmaceutical composition as provided herein to a subject twice a day, daily, every other day, three times a week, twice a week, weekly, every other week, twice a month, or monthly.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGURES, and from the claims.

### BRIEF DESCRIPTION OF THE FIGURES

FIG. 1 provides an outline for a study to assess the effects of treatment with CHS-131 (Compound of Formula (I)), alone and in combination with other therapeutic agents, to treat NASH, as described in Example 3.

### DETAILED DESCRIPTION

### Definitions

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 up to and including 22.

The term “administration” or “administering” refers to a method of giving a dosage of a compound or pharmaceutical composition to a vertebrate or invertebrate, including a mammal, a bird, a fish, or an amphibian. The preferred method of administration can vary depending on various factors, e.g., the components of the pharmaceutical composition, the site of the disease, and the severity of the disease.

The term “CHS-131” as used herein refers to a compound of Formula (I):

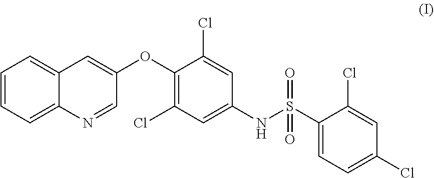 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
The compound of Formula (I) is a selective peroxisome proliferator-activated receptor (PPAR) γ modulator. The compound of Formula (I) is disclosed in, for example, U.S. Pat. Nos. 7,041,691; 6,200,995; 6,583,157; 6,653,332; and U.S. Publication Application No. 2016/0260398, the contents of each of which are incorporated by reference herein in their entireties.

The compound of Formula (I) can be prepared, for example, by the methods described in U.S. Pat. Nos. 6,583,157 or 6,200,995, each of which is incorporated by reference in its entirety herein. In some embodiments, different salts, e.g., besylate, tosylate HCl, or HBr salts, and/or polymorphs of the compound of Formula (I) are used within the methods and compositions described herein. Salts and polymorphs of the compound of Formula (I), such as those provided herein, can be prepared according to the methods described in U.S. Pat. Nos. 6,583,157 and 7,223,761, the contents of each of which are incorporated by reference in their entireties.

In some embodiments described herein, the compound of Formula I is a free base. In other embodiments, the compound of Formula I is a pharmaceutically acceptable salt, for example a hydrochloride or besylate salt.

The term “ASK1 inhibitor” as used herein refers to a compound that inhibits the activity of apoptosis signal-regulating kinase 1 (ASK1). Examples of ASK1 inhibitors include, but are not limited to:

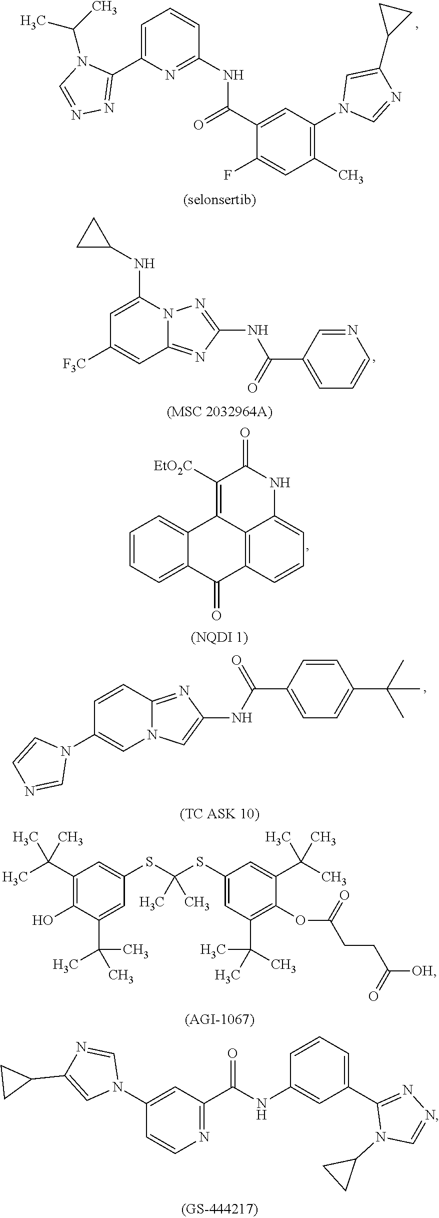

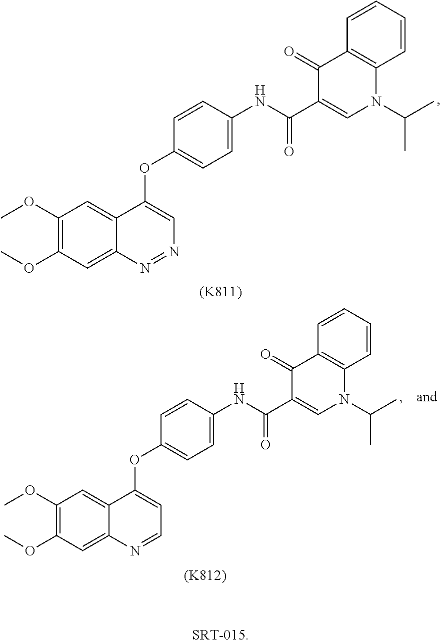

The term “ACC inhibitor” as used herein refers to a compound that inhibitors the activity of one or more ACC enzymes. In some embodiments, the ACC inhibitor is a compound that selectively inhibits the activity of ACC1. In other embodiments, the ACC inhibitor is a compound that selectively inhibits the activity of ACC2. In yet still other embodiments, the ACC inhibitor is a compound that inhibits the activity of both ACC1 and ACC2 (e.g., a dual, or non-selective, ACC inhibitor). Examples of ACC inhibitors include, but are not limited to:

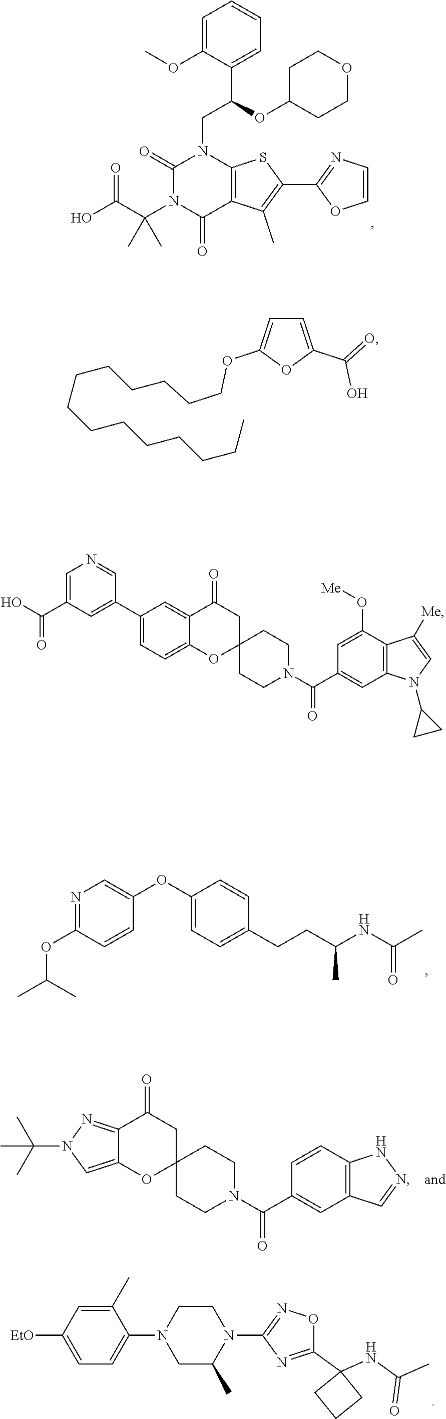

The term “caspase inhibitor” as used herein refers to a compound that inhibits the activity of one or more caspases. For example, one or more of caspase 1, 2, 3, 4, 5, 6, 7, 8, 9, 10, 12, and 14. Caspase inhibitors include, but are not limited to

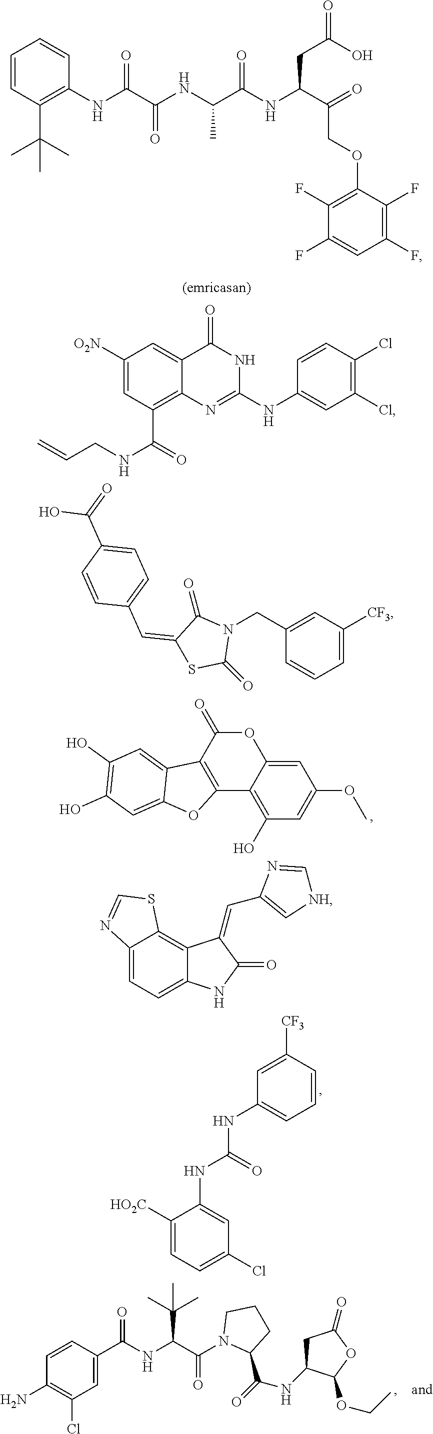

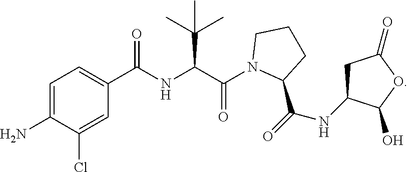 [SMILES: CC(C)(C)[C@H](NC(=O)c1ccc(N)c(Cl)c1)C(=O)N1CCC[C@H]1C(=O)N[C@H]1CC(=O)O[C@H]1O]

The term “mineralocorticoid receptor antagonist” (MCR antagonist) as used herein refers to a compound that binds to decreases the activation of the mineralocorticoid receptor. Examples of MCR antagonists include, but are not limited to spironolactone, finerenone, eplerenone, canrenone, mexrenone, amlodipine, apararenone, benidipine, esaxerenone, felodipine, nefedipine, nimodipine, and nitrendipine.

The term “statin,” as used herein refers to a compound that inhibits the activity of HMG-CoA reductase. Exemplary statins include, but are not limited to, atorvastatin, cerivastatin, fluvastatin, lovastatin, mevastatin, pitavastatin, pravastatin, rosuvastatin, and simvastatin.

The term “PCSK9 inhibitor” as used herein refers to a compound that inhibits the activity of PCSK9. Examples of PCSK9 inhibitors include, but are not limited to, alirocumab, evolocumab, bococizumab, 1D05-IgG2, RG-7652, LY3015014, and inclisiran.

In some embodiments, the ASK1 inhibitor is a free base. In other embodiments, the ASK1 inhibitor is a pharmaceutically acceptable salt, for example, a hydrochloride salt or a sodium salt. In some embodiments, the ACC inhibitor is a free base. In other embodiments, the ACC inhibitor is a pharmaceutically acceptable salt, for example, a hydrochloride salt or a sodium salt. In some embodiments, the caspase inhibitor is a free base. In other embodiments, the caspase inhibitor is a pharmaceutically acceptable salt, for example, a hydrochloride salt or a sodium salt. In some embodiments, the MCR antagonist is a free base. In other embodiments, the MCR antagonist is a pharmaceutically acceptable salt, for example, a hydrochloride salt or a sodium salt. In some embodiments, the statin is a free base. In other embodiments, the statin is a pharmaceutically acceptable salt, for example, a hydrochloride salt or a sodium salt. In some embodiments, the PCSK9 inhibitor is a free base. In other embodiments, the PCSK9 inhibitor is a pharmaceutically acceptable salt, for example, a hydrochloride salt or a sodium salt.

By “effective dosage” or “therapeutically effective amount” or “pharmaceutically effective amount” of a compound as provided herein is an amount that is sufficient to achieve the desired therapeutic effect and can vary according to the nature and severity of the disease condition, and the potency of the compound. In some embodiments, the therapeutic effect is determined from one or more parameters selected from the NAFLD Activity Score (NAS), hepatic steatosis, hepatic inflammation, biomarkers indicative of liver damage, and liver fibrosis and/or liver cirrhosis. For example, a therapeutic effect can include one or more of a decrease in symptoms, a decrease in the NAS, a reduction in the amount of hepatic steatosis, a decrease in hepatic inflammation, a decrease in the level of biomarkers indicative of liver damage, and a reduction in liver fibrosis and/or liver cirrhosis, a lack of further progression of liver fibrosis and/or liver cirrhosis, or a slowing of the progression of liver fibrosis and/or liver cirrhosis following administration of a compound or compounds as described herein.

A “therapeutic effect,” as used herein, refers to the relief, to some extent, of one or more of the symptoms of the disease, and can include curing a disease. “Curing” means that the symptoms of active disease are eliminated. However, certain long-term or permanent effects of the disease can exist even after a cure is obtained (such as, e.g., extensive tissue damage). In some embodiments, a therapeutically effective amount of a compound as provided herein refers to an amount of the compound that is effective as a monotherapy.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g., side effects and adverse events) of at least one of the therapeutic agents.

For example, a synergistic ratio of two therapeutic agents can be identified by determining a synergistic effect in, for example, an art-accepted in vivo model (e.g., an animal model) of NAFLD (e.g., the diet induced obese (DIO)-NASH mouse model or any of the models described in Van Herck et al. Nutrients. 2017 October; 9(10): 1072, and Kristiansen et al. World J Hepatol. 2016; 8(16):673-84, which are incorporated by reference herein in their entirety). In one embodiment of a DIO-NASH model, the mouse model is induced by feeding male C57BL/6JRj mice a high fat diet containing 40% fat with trans-fat, 20% fructose and 2% cholesterol (AMLN diet or D09100301, Research Diets Inc., USA). In another embodiment, the model is a male Lep^{ob}/Lep^{ob} (ob/ob) mouse model.

The term “preventing” as used herein means the prevention of the onset, recurrence or spread, in whole or in part, of the disease or condition as described herein, or a symptom thereof.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “subject” or “patient” refers to any subject, particularly a mammalian subject, for whom diagnosis, prognosis, or therapy is desired, for example, a human.

The terms “treatment regimen” and “dosing regimen” are used interchangeably to refer to the dose and timing of administration of each therapeutic agent in a combination of the invention.

The term “pharmaceutical combination”, as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination) (e.g., the compound of Formula (I), or a pharmaceutically acceptable salt thereof, and an additional therapeutic agent), wherein the therapeutically active agents are administered together or separately in a manner prescribed by a medical care taker or according to a regulatory agency as defined herein. The “additional therapeutic agent” refers to those classes of compounds described herein for use with the compound Formula (I) (e.g., ASK1 inhibitors, ACC inhibitors, caspase inhibitors, MCR antagonists, statins, and PCSK9 inhibitors.

In one embodiment, a combination therapy comprises a combination of the compound of Formula (I), or a pharmaceutically acceptable salt thereof, and an ASK1 inhibitor (e.g., selonsertib, MSC 2032964A, NQDI 1, TC ASK 10, AGI-1067, GS-444217, K811, K812, or SRT-015), or a pharmaceutically acceptable salt thereof. In one embodiment, a combination therapy consists essentially of a combination of (a) the compound of Formula (I), or a pharmaceutically acceptable salt thereof, and (b) an ASK1 inhibitor, or a pharmaceutically acceptable salt thereof (e.g., selonsertib, MSC 2032964A, NQDI 1, TC ASK 10, AGI-1067, GS-444217, K811, K812, or SRT-015).

In one embodiment, a combination therapy comprises a combination of the compound of Formula (I), or a pharmaceutically acceptable salt thereof, and ACC inhibitor (such as those described herein), or a pharmaceutically acceptable salt thereof. In one embodiment, a combination therapy consists essentially of a combination of (a) the compound of Formula (I), or a pharmaceutically acceptable salt thereof, and (b) an ACC inhibitor, or a pharmaceutically acceptable salt thereof (such as those described herein).

In one embodiment, a combination therapy comprises a combination of the compound of Formula (I), or a pharmaceutically acceptable salt thereof, and a caspase inhibitor (such as those described herein), or a pharmaceutically acceptable salt thereof. In one embodiment, a combination therapy consists essentially of a combination of (a) the compound of Formula (I), or a pharmaceutically acceptable salt thereof, and (b) a caspase inhibitor, or a pharmaceutically acceptable salt thereof (such as those described herein).

In one embodiment, a combination therapy comprises a combination of the compound of Formula (I), or a pharmaceutically acceptable salt thereof, and an MCR antagonist (e.g., spironolactone, finerenone, eplerenone, canrenone, mexrenone, amlodipine, apararenone, benidipine, esaxerenone, felodipine, nefedipine, nimodipine, and nitrendipine), or a pharmaceutically acceptable salt thereof. In one embodiment, a combination therapy consists essentially of a combination of (a) the compound of Formula (I), or a pharmaceutically acceptable salt thereof, and (b) an MCR antagonist, or a pharmaceutically acceptable salt thereof (e.g., spironolactone, finerenone, eplerenone, canrenone, mexrenone, amlodipine, apararenone, benidipine, esaxerenone, felodipine, nefedipine, nimodipine, and nitrendipine).

In one embodiment, a combination therapy comprises a combination of the compound of Formula (I), or a pharmaceutically acceptable salt thereof, and statin (e.g., atorvastatin, cerivastatin, fluvastatin, lovastatin, mevastatin, pitavastatin, pravastatin, rosuvastatin, or simvastatin), or a pharmaceutically acceptable salt thereof. In one embodiment, a combination therapy consists essentially of a combination of (a) the compound of Formula (I), or a pharmaceutically acceptable salt thereof, and (b) a statin, or a pharmaceutically acceptable salt thereof (e.g., atorvastatin, cerivastatin, fluvastatin, lovastatin, mevastatin, pitavastatin, pravastatin, rosuvastatin, or simvastatin).

In one embodiment, a combination therapy comprises a combination of the compound of Formula (I), or a pharmaceutically acceptable salt thereof, and a PCSK9 inhibitor (e.g., alirocumab, evolocumab, bococizumab, 1D05-IgG2, RG-7652, LY3015014, and inclisiran). In one embodiment, a combination therapy consists essentially of a combination of (a) the compound of Formula (I), or a pharmaceutically acceptable salt thereof, and (b) a PCSK9 inhibitor (e.g., alirocumab, evolocumab, bococizumab, 1D05-IgG2, RG-7652, LY3015014, and inclisiran).

The term “fixed combination” means that the compound of Formula (I), or a pharmaceutically acceptable salt thereof, and an additional therapeutic agent as described herein, are each administered to a subject simultaneously in the form of a single composition or dosage.

The term “non-fixed combination” means that the compound of Formula (I), or a pharmaceutically acceptable salt thereof, and an additional therapeutic agent as described herein, are formulated as separate compositions or dosages such that they may be administered to a subject in need thereof concurrently or sequentially with variable intervening time limits, wherein such administration provides effective levels of the two or more compounds in the body of the subject. These also apply to cocktail therapies, e.g., the administration of three or more active ingredients.

As can be appreciated in the art, a combination therapy can be administered to a patient for a period of time. In some embodiments, the period of time occurs following the administration of a different therapeutic treatment/agent or a different combination of therapeutic treatments/agents to the patient. In some embodiments, the period of time occurs before the administration of a different therapeutic treatment/agent or a different combination of therapeutic treatments/agents to the subject.

A suitable period of time can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. In some embodiments, a suitable period of time can be from 1 week to 2 years, for example, 1 week, 2, weeks, 4 weeks, 6 weeks, 8 weeks, 12 weeks, 16 weeks, 6 months, 9 months, 12 months, 18 months, or 2 years, or any value in between. In other embodiments, a suitable period of time can be from 1 month to 10 years, for example, 1 month, 6 months, 1 year, 18 months, 2 years, 3 years, 4 years, 5 years, 6 years, 7 years, 8 years, 9 years, or 10 years, or any value in between

The phrases “prior to a period of time” or “before a period of time” refer to (1) the completion of administration of treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

In some embodiments, a combination of (a) the compound of Formula (I), or a pharmaceutically acceptable salt thereof, and (b) an ASK1 inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the ASK1 inhibitor, or a pharmaceutically acceptable salt thereof are each administered alone.

In some embodiments, a combination of (a) the compound of Formula (I), or a pharmaceutically acceptable salt thereof, and (b) an ASK1 inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the ASK1 inhibitor, or a pharmaceutically acceptable salt thereof as in the combination are each administered alone.

In some more particular embodiments a combination of (a) the compound of Formula (I), or a pharmaceutically acceptable salt thereof, and (b) an ASK1 inhibitor, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments, the amounts of the two or more compounds as provided herein together are effective in treating NAFLD (e.g., the amounts of the compound of Formula (I) and an ASK1 inhibitor together are effective in treating NAFLD). For example, wherein the amounts of (a) the compound of Formula (I), or a pharmaceutically acceptable salt thereof, (b) an ASK1 inhibitor, or a pharmaceutically acceptable salt thereof, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some embodiments, a combination of (a) the compound of Formula (I), or a pharmaceutically acceptable salt thereof, and (b) an ACC inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the ACC inhibitor, or a pharmaceutically acceptable salt thereof are each administered alone.

In some embodiments, a combination of (a) the compound of Formula (I), or a pharmaceutically acceptable salt thereof, and (b) an ACC inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the ACC inhibitor, or a pharmaceutically acceptable salt thereof as in the combination are each administered alone.

In some more particular embodiments a combination of (a) the compound of Formula (I), or a pharmaceutically acceptable salt thereof, and (b) an ACC inhibitor, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments, the amounts of the two or more compounds as provided herein together are effective in treating NAFLD (e.g., the amounts of the compound of Formula (I) and An ACC inhibitor together are effective in treating NAFLD). For example, wherein the amounts of (a) the compound of Formula (I), or a pharmaceutically acceptable salt thereof, (b) An ACC inhibitor, or a pharmaceutically acceptable salt thereof, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some embodiments, a combination of (a) the compound of Formula (I), or a pharmaceutically acceptable salt thereof, and (b) a caspase inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the caspase inhibitor, or a pharmaceutically acceptable salt thereof are each administered alone.

In some embodiments, a combination of (a) the compound of Formula (I), or a pharmaceutically acceptable salt thereof, and (b) an caspase inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the caspase inhibitor, or a pharmaceutically acceptable salt thereof as in the combination are each administered alone.

In some more particular embodiments a combination of (a) the compound of Formula (I), or a pharmaceutically acceptable salt thereof, and (b) a caspase inhibitor, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments, a combination of (a) the compound of Formula (I), or a pharmaceutically acceptable salt thereof, and (b) an MCR antagonist, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the MCR antagonist, or a pharmaceutically acceptable salt thereof are each administered alone.

In some embodiments, a combination of (a) the compound of Formula (I), or a pharmaceutically acceptable salt thereof, and (b) an MCR antagonist, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the MCR antagonist, or a pharmaceutically acceptable salt thereof as in the combination are each administered alone.

In some more particular embodiments a combination of (a) the compound of Formula (I), or a pharmaceutically acceptable salt thereof, and (b) an MCR antagonist, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments, a combination of (a) the compound of Formula (I), or a pharmaceutically acceptable salt thereof, and (b) a statin, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the statin, or a pharmaceutically acceptable salt thereof are each administered alone.

In some embodiments, a combination of (a) the compound of Formula (I), or a pharmaceutically acceptable salt thereof, and (b) a statin, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the statin, or a pharmaceutically acceptable salt thereof as in the combination are each administered alone.

In some more particular embodiments a combination of (a) the compound of Formula (I), or a pharmaceutically acceptable salt thereof, and (b) a statin, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments, a combination of (a) the compound of Formula (I), or a pharmaceutically acceptable salt thereof, and (b) a PCSK9 inhibitor, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the PCSK9 inhibitor, are each administered alone.

In some embodiments, a combination of (a) the compound of Formula (I), or a pharmaceutically acceptable salt thereof, and (b) a PCSK9 inhibitor, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the PCSK9 inhibitor, or a pharmaceutically acceptable salt thereof as in the combination are each administered alone.

In some more particular embodiments a combination of (a) the compound of Formula (I), or a pharmaceutically acceptable salt thereof, and (b) a PCSK9 inhibitor, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments of the methods or combinations herein, a subject may be administered an amount of a compound that produces a therapeutic effect in the absence of another compound of the combinations disclosed herein. In particular embodiments of the methods or combinations herein, a subject may be administered two compounds which together produce a therapeutic effect. For example, two compounds when dosed together may have an additive or synergistic effect, such that the dose of each individual compound may independently be an effective amount, or may be a sub-therapeutic amount, but together the total amount of the combination of compounds provides a therapeutically effective amount.

In some more particular embodiments, a combination of (a) the compound of Formula (I), or a pharmaceutically acceptable salt thereof, and (b) an ASK1 inhibitor, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) an ASK1 inhibitor, or a pharmaceutically acceptable salt thereof, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, an ASK1 inhibitor, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or an ASK1 inhibitor, or a pharmaceutically acceptable salt thereof. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

In some more particular embodiments, a combination of (a) the compound of Formula (I), or a pharmaceutically acceptable salt thereof, and (b) an ACC inhibitor, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) an ACC inhibitor, or a pharmaceutically acceptable salt thereof, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, an ACC inhibitor, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or an ACC inhibitor, or a pharmaceutically acceptable salt thereof. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

In some embodiments, the amounts of the two or more compounds as provided herein together are effective in treating NAFLD (e.g., the amounts of the compound of Formula (I) and a caspase inhibitor together are effective in treating NAFLD). For example, wherein the amounts of (a) the compound of Formula (I), or a pharmaceutically acceptable salt thereof, (b) a caspase inhibitor, or a pharmaceutically acceptable salt thereof, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some more particular embodiments, a combination of (a) the compound of Formula (I), or a pharmaceutically acceptable salt thereof, and (b) a caspase inhibitor, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) a caspase inhibitor, or a pharmaceutically acceptable salt thereof, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, a caspase inhibitor, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or a caspase inhibitor, or a pharmaceutically acceptable salt thereof. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

In some embodiments, the amounts of the two or more compounds as provided herein together are effective in treating NAFLD (e.g., the amounts of the compound of Formula (I) and an MCR antagonist together are effective in treating NAFLD). For example, wherein the amounts of (a) the compound of Formula (I), or a pharmaceutically acceptable salt thereof, (b) an MCR antagonist, or a pharmaceutically acceptable salt thereof, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some more particular embodiments, a combination of (a) the compound of Formula (I), or a pharmaceutically acceptable salt thereof, and (b) an MCR antagonist, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) an MCR antagonist, or a pharmaceutically acceptable salt thereof, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, an MCR antagonist, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or an MCR antagonist, or a pharmaceutically acceptable salt thereof. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

In some embodiments, the amounts of the two or more compounds as provided herein together are effective in treating NAFLD (e.g., the amounts of the compound of Formula (I) and a statin together are effective in treating NAFLD). For example, wherein the amounts of (a) the compound of Formula (I), or a pharmaceutically acceptable salt thereof, (b) a statin, or a pharmaceutically acceptable salt thereof, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a a pharmaceutically acceptable salt thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some more particular embodiments, a combination of (a) the compound of Formula (I), or a pharmaceutically acceptable salt thereof, and (b) a statin, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) a statin, or a pharmaceutically acceptable salt thereof, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, a statin, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or a statin, or a pharmaceutically acceptable salt thereof. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

In some embodiments, the amounts of the two or more compounds as provided herein together are effective in treating NAFLD (e.g., the amounts of the compound of Formula (I) and a PCSK9 inhibitor together are effective in treating NAFLD). For example, wherein the amounts of (a) the compound of Formula (I), or a pharmaceutically acceptable salt thereof, (b) a PCSK9 inhibitor, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some more particular embodiments, a combination of (a) the compound of Formula (I), or a pharmaceutically acceptable salt thereof, and (b) a PCSK9 inhibitor, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) a PCSK9 inhibitor, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, a PCSK9 inhibitor, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or a PCSK9 inhibitor. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

### Methods and Combination Therapies

The combination therapies described herein refer to an (a) and a (b) component of the combination. Unless expressly stated otherwise, (a) refers to the compound of Formula (I), or a pharmaceutically acceptable salt thereof and (b) refers to any of the ASK1 inhibitors, ACC inhibitors, caspase inhibitors, MCR antagonists, statins, or PCSK9 inhibitors described herein.

The present disclosure relates to methods and combination therapies for treating non-alcoholic fatty liver disease (NAFLD) in a subject in need thereof by administering (a) the compound of Formula (I):

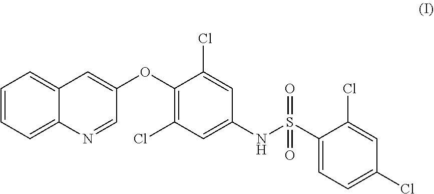 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) an ASK1 inhibitor, or a pharmaceutically acceptable salt thereof.

The present disclosure also relates to methods and combination therapies for treating non-alcoholic fatty liver disease (NAFLD) in a subject in need thereof by administering (a) the compound of Formula (I):

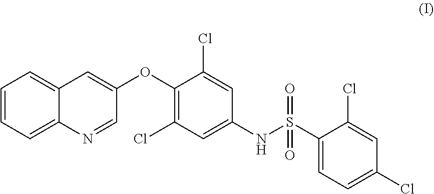 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) an ACC inhibitor, or a pharmaceutically acceptable salt thereof.

The present disclosure also relates to methods and combination therapies for treating non-alcoholic fatty liver disease (NAFLD) in a subject in need thereof by administering (a) the compound of Formula (I):

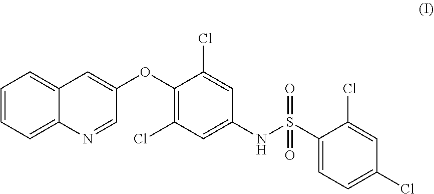 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) an caspase inhibitor, or a pharmaceutically acceptable salt thereof.

The present disclosure also relates to methods and combination therapies for treating non-alcoholic fatty liver disease (NAFLD) in a subject in need thereof by administering (a) the compound of Formula (I):

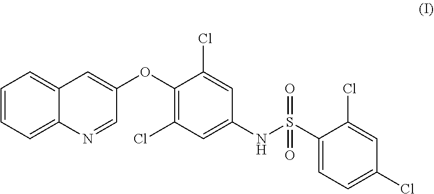 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) an MCR antagonist, or a pharmaceutically acceptable salt thereof.

The present disclosure also relates to methods and combination therapies for treating non-alcoholic fatty liver disease (NAFLD) in a subject in need thereof by administering (a) the compound of Formula (I):

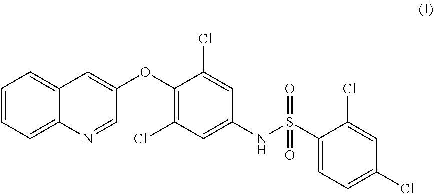 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) a statin, or a pharmaceutically acceptable salt thereof.

The present disclosure also relates to methods and combination therapies for treating non-alcoholic fatty liver disease (NAFLD) in a subject in need thereof by administering (a) the compound of Formula (I):

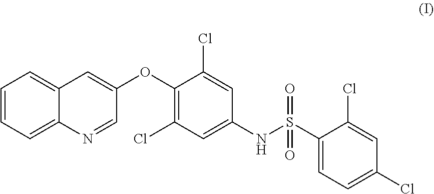 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) a PCSK9 inhibitor, or a pharmaceutically acceptable salt thereof.

NAFLD is characterized by hepatic steatosis with no secondary causes of hepatic steatosis including excessive alcohol consumption, other known liver diseases, or long-term use of a steatogenic medication (Chalasani et al., Hepatology. 2018, 67(1):328-357, which is hereby incorporated by reference in its entirety). NAFLD can be categorized into non-alcoholic fatty liver (NAFL) and non-alcoholic steatohepatitis (NASH). According to Chalasani et al., NAFL is defined as the presence of ≥5% hepatic steatosis without evidence of hepatocellular injury in the form of hepatocyte ballooning. NASH is defined as the presence of ≥5% hepatic steatosis and inflammation with hepatocyte injury (e.g., ballooning), with or without any liver fibrosis. Additionally, NASH is commonly associated with hepatic inflammation and liver fibrosis, which can progress to cirrhosis, end-stage liver disease, and hepatocellular carcinoma. However, liver fibrosis is not always present in NASH, but the severity of fibrosis can be linked to long-term outcomes.

There are many approaches used to assess and evaluate whether a subject has NAFLD and if so, the severity of the disease including differentiating whether the NAFLD is NAFL or NASH. For example, these approaches include determining one or more of hepatic steatosis (e.g., accumulation of fat in the liver); the NAFLD Activity Score (NAS); hepatic inflammation; biomarkers indicative of one or more of liver damage, hepatic inflammation, liver fibrosis, and/or liver cirrhosis (e.g., serum markers and panels); and liver fibrosis and/or cirrhosis. Further examples of physiological indicators of NAFLD can include liver morphology, liver stiffness, and the size or weight of the subject's liver. In some embodiments, NAFLD in the subject is evidenced by an accumulation of hepatic fat and detection of a biomarker indicative of liver damage. For example, elevated serum ferritin and low titers of serum autoantibodies can be common features of NAFLD. In some embodiments, methods to assess NAFLD include magnetic resonance imaging, either by spectroscopy or by proton density fat fraction (MRI-PDFF) to quantify steatosis, transient elastography (FIBROSCAN®), hepatic venous pressure gradient (HPVG), hepatic stiffness measurement with MRE for diagnosing significant liver fibrosis and/or cirrhosis, and assessing histological features of liver biopsy. In some embodiments, magnetic resonance imaging is used to detect one or more of steatohepatitis (NASH-MRI), liver fibrosis (Fibro-MRI), and steatosis see, for example, U.S. Application Publication Nos. 2016/146715 and 2005/0215882, each of which are incorporated herein by reference in their entireties. In some embodiments, treatment of NAFLD comprises one or more of a decrease in symptoms; a reduction in the amount of hepatic steatosis; a decrease in the NAS; a decrease in hepatic inflammation; a decrease in the level of biomarkers indicative of one or more of liver damage, inflammation, liver fibrosis, and/or liver cirrhosis; and a reduction in fibrosis and/or cirrhosis, a lack of further progression of fibrosis and/or cirrhosis, or a slowing of the progression of fibrosis and/or cirrhosis.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, hepatic steatosis is determined by one or more methods selected from the group consisting of ultrasonography, computed tomography (CT), magnetic resonance imaging, magnetic resonance spectroscopy (MRS), magnetic resonance elastography (MRE), transient elastography (TE) (e.g., FIBROSCAN®), measurement of liver size or weight, or by liver biopsy (see, e.g., Di Lascio et al., Ultrasound Med Biol. 2018 August; 44(8):1585-1596; Lv et al., J Clin Transl Hepatol. 2018 Jun. 28; 6(2): 217-221; Reeder, et al., J Magn Reson Imaging. 2011 October; 34(4): 848-855; and de Lédinghen V, et al., J Gastroenterol Hepatol. 2016 April; 31(4):848-55, each of which are incorporated herein by reference in their entireties). A subject diagnosed with NAFLD can have more than about 5% hepatic steatosis, for example, about 5% to about 25%, about 25% to about 45%, about 45% to about 65%, or greater than about 65% hepatic steatosis. In some embodiments, a subject with about 5% to about 33% hepatic steatosis has stage 1 hepatic steatosis, a subject with about 33% to about 66% hepatic steatosis has stage 2 hepatic steatosis, and a subject with greater than about 66% hepatic steatosis has stage 3 hepatic steatosis. In some embodiments, treatment of NAFLD can be assessed by measuring hepatic steatosis. In some embodiments, treatment of NAFLD comprises a reduction in hepatic steatosis following administration of one or more compounds described herein.

In some embodiments, the amount of hepatic steatosis is determined prior to administration of the combination of (a) the compound of Formula (I), or a pharmaceutically acceptable salt thereof, and (b) an ASK1 inhibitor, or a pharmaceutically acceptable salt thereof. In some embodiments, the amount of hepatic steatosis is determined prior to administration of the combination of (a) the compound of Formula (I), or a pharmaceutically acceptable salt thereof, and (b) an ACC inhibitor, or a pharmaceutically acceptable salt thereof. In some embodiments, the amount of hepatic steatosis is determined prior to administration of the combination of (a) the compound of Formula (I), or a pharmaceutically acceptable salt thereof, and (b) an caspase inhibitor, or a pharmaceutically acceptable salt thereof. In some embodiments, the amount of hepatic steatosis is determined prior to administration of the combination of (a) the compound of Formula (I), or a pharmaceutically acceptable salt thereof, and (b) an MCR antagonist, or a pharmaceutically acceptable salt thereof. In some embodiments, the amount of hepatic steatosis is determined prior to administration of the combination of (a) the compound of Formula (I), or a pharmaceutically acceptable salt thereof, and (b) a statin, or a pharmaceutically acceptable salt thereof. In some embodiments, the amount of hepatic steatosis is determined prior to administration of the combination of (a) the compound of Formula (I), or a pharmaceutically acceptable salt thereof, and (b) a PCSK9 inhibitor, or a pharmaceutically acceptable salt thereof.

In some embodiments, the amount of hepatic steatosis is determined during the period of time or after the period of time of administration of the combination of (a) and (b). In some embodiments, a reduction in the amount of hepatic steatosis during the period of time or after the period of time of administration of the combination of (a) and (b) compared to prior to administration of the combination of (a) and (b) indicates treatment of NAFLD. For example, a reduction in the amount of hepatic steatosis by about 1% to about 50%, about 25% to about 75%, or about 50% to about 100% indicates treatment of NAFLD. In some embodiments, a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, the severity of NALFD can be assessed using the NAS. In some embodiments, treatment of NAFLD can be assessed using the NAS. In some embodiments, treatment of NAFLD comprises a reduction in the NAS following administration of one or more compounds described herein. In some embodiments, the NAS can be determined as described in Kleiner et al., Hepatology. 2005, 41(6):1313-1321, which is hereby incorporated by reference in its entirety. See, for example, Table 2 for a simplified NAS scheme adapted from Kleiner.

TABLE 2
Example of the NAFLD
Activity Score (NAS) with Fibrosis Stage

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| Inflammation | <2 foci/200x | 1 |
| | 2-4 foci/200x | 2 |
| | >4 foci/200x | 3 |
| Ballooning | None | 0 |
| degeneration | Few | 1 |
| | Many cells/Prominent | 2 |
| | ballooning | |
| Fibrosis | None | 0 |
| | Perisinusoidal or | 1 |
| | periportal | |
| | Perisinusoidal & | 2 |
| | portal/periportal | |
| | Bridging fibrosis | 3 |
| | Cirrhosis | 4 |

In some embodiments, the NAS is determined non-invasively, for example, as described in U.S. Application Publication No. 2018/0140219, which is incorporated by reference herein in its entirety. In some embodiments, the NAS is determined for a sample from the subject prior to administration of the combination of (a) the compound of Formula (I), or a pharmaceutically acceptable salt thereof, and (b) an ASK1 inhibitor, or a pharmaceutically acceptable salt thereof. In some embodiments, the NAS is determined for a sample from the subject prior to administration of the combination of (a) the compound of Formula (I), or a pharmaceutically acceptable salt thereof, and (b) an ACC inhibitor, or a pharmaceutically acceptable salt thereof. In some embodiments, the NAS is determined for a sample from the subject prior to administration of the combination of (a) the compound of Formula (I), or a pharmaceutically acceptable salt thereof, and (b) a caspase inhibitor, or a pharmaceutically acceptable salt thereof. In some embodiments, the NAS is determined for a sample from the subject prior to administration of the combination of
(a) the compound of Formula (I), or a pharmaceutically acceptable salt thereof, and (b) an MCR antagonist, or a pharmaceutically acceptable salt thereof. In some embodiments, the NAS is determined for a sample from the subject prior to administration of the combination of (a) the compound of Formula (I), or a pharmaceutically acceptable salt thereof, and (b) a statin, or a pharmaceutically acceptable salt thereof. In some embodiments, the NAS is determined for a sample from the subject prior to administration of the combination of (a) the compound of Formula (I), or a pharmaceutically acceptable salt thereof, and (b) an PCSK9 inhibitor, or a pharmaceutically acceptable salt thereof.

In some embodiments, the NAS is determined during the period of time or after the period of time of administration of the combination of (a) and (b). In some embodiments, a lower NAS score during the period of time or after the period of time of administration of the combination of (a) and (b) compared to prior to administration of the combination of (a) and (b) indicates treatment of NAFLD. For example, a decrease in the NAS by 1, by 2, by 3, by 4, by 5, by 6, or by 7 indicates treatment of NAFLD. In some embodiments, the NAS following administration of the combination of (a) and (b) is 7 or less. In some embodiments, the NAS during the period of time of administration of the combination of (a) and (b) is 5 or less, 4 or less, 3 or less, or 2 or less. In some embodiments, the NAS during the period of time of administration of the combination of (a) and (b) is 7 or less. In some embodiments, the NAS during the period of time of administration of the combination of (a) and (b) is 5 or less, 4 or less, 3 or less, or 2 or less. In some embodiments, the NAS after the period of time of administration of the combination of (a) and (b) is 7 or less. In some embodiments, the NAS after the period of time of administration of the combination of (a) and (b) is 5 or less, 4 or less, 3 or less, or 2 or less.

In some embodiments, the presence of hepatic inflammation is determined by one or more methods selected from the group consisting of biomarkers indicative of hepatic inflammation and a liver biopsy sample(s) from the subject. In some embodiments, the severity of hepatic inflammation is determined from a liver biopsy sample(s) from the subject. For example, hepatic inflammation in a liver biopsy sample can be assessed as described in Kleiner et al., Hepatology. 2005, 41(6):1313-1321 and Brunt et al., Am J Gastroenterol 1999, 94:2467-2474, each of which are hereby incorporated by reference in their entireties.

In some embodiments, the severity of hepatic inflammation is determined prior to administration of the combination of (a) the compound of Formula (I), or a pharmaceutically acceptable salt thereof, and (b) an ASK1 inhibitor, or a pharmaceutically acceptable salt thereof. In some embodiments, the severity of hepatic inflammation is determined prior to administration of the combination of (a) the compound of Formula (I), or a pharmaceutically acceptable salt thereof, and (b) an ASK1 inhibitor, or a pharmaceutically acceptable salt thereof.

In some embodiments, the severity of hepatic inflammation is determined prior to administration of the combination of (a) the compound of Formula (I), or a pharmaceutically acceptable salt thereof, and (b) an ACC inhibitor, or a pharmaceutically acceptable salt thereof. In some embodiments, the severity of hepatic inflammation is determined prior to administration of the combination of (a) the compound of Formula (I), or a pharmaceutically acceptable salt thereof, and (b) an ACC inhibitor, or a pharmaceutically acceptable salt thereof.

In some embodiments, the severity of hepatic inflammation is determined prior to administration of the combination of (a) the compound of Formula (I), or a pharmaceutically acceptable salt thereof, and (b) a caspase inhibitor, or a pharmaceutically acceptable salt thereof. In some embodiments, the severity of hepatic inflammation is determined prior to administration of the combination of (a) the compound of Formula (I), or a pharmaceutically acceptable salt thereof, and (b) a caspase inhibitor, or a pharmaceutically acceptable salt thereof.

In some embodiments, the severity of hepatic inflammation is determined prior to administration of the combination of (a) the compound of Formula (I), or a pharmaceutically acceptable salt thereof, and (b) MCR antagonist, or a pharmaceutically acceptable salt thereof. In some embodiments, the severity of hepatic inflammation is determined prior to administration of the combination of (a) the compound of Formula (I), or a pharmaceutically acceptable salt thereof, and (b) a MCR antagonist, or a pharmaceutically acceptable salt thereof.

In some embodiments, the severity of hepatic inflammation is determined prior to administration of the combination of (a) the compound of Formula (I), or a pharmaceutically acceptable salt thereof, and (b) a statin, or a pharmaceutically acceptable salt thereof. In some embodiments, the severity of hepatic inflammation is determined prior to administration of the combination of (a) the compound of Formula (I), or a pharmaceutically acceptable salt thereof, and (b) a statin, or a pharmaceutically acceptable salt thereof.

In some embodiments, the severity of hepatic inflammation is determined prior to administration of the combination of (a) the compound of Formula (I), or a pharmaceutically acceptable salt thereof, and (b) a PCSK9 inhibitor, or a pharmaceutically acceptable salt thereof. In some embodiments, the severity of hepatic inflammation is determined prior to administration of the combination of (a) the compound of Formula (I), or a pharmaceutically acceptable salt thereof, and (b) a PCSK9 inhibitor, or a pharmaceutically acceptable salt thereof.

In some embodiments, the severity of hepatic inflammation is determined during the period of time or after the period of time of administration of the combination of (a) and (b). In some embodiments, a decrease in the severity of hepatic inflammation during the period of time or after the period of time of administration of the combination of (a) and (b) compared to prior to administration of the combination of (a) and (b) indicates treatment of NAFLD. For example, a decrease in the severity of hepatic inflammation by about 1% to about 50%, about 25% to about 75%, or about 50% to about 100% indicates treatment of NAFLD. In some embodiments, a decrease in the severity of hepatic inflammation by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, treatment of NAFLD comprises treatment of fibrosis and/or cirrhosis, e.g., a decrease in the severity of fibrosis, a lack of further progression of fibrosis and/or cirrhosis, or a slowing of the progression of fibrosis and/or cirrhosis. In some embodiments, the presence of fibrosis and/or cirrhosis is determined by one or more methods selected from the group consisting of transient elastography (e.g., FIBROSCAN®), non-invasive markers of hepatic fibrosis, and histological features of a liver biopsy. In some embodiments, the severity (e.g., stage) of fibrosis is determined by one or more methods selected from the group consisting of transient elastography (e.g., FIBROSCAN®), a fibrosis-scoring system, biomarkers of hepatic fibrosis (e.g., non-invasive biomarkers), and hepatic venous pressure gradient (HVPG). Non-limiting examples of fibrosis scoring systems include the NAFLD fibrosis scoring system (see, e.g., Angulo, et al., Hepatology. 2007; 45(4):846-54), the fibrosis scoring system in Brunt et al., Am J Gastroenterol. 1999, 94:2467-2474, the fibrosis scoring system in Kleiner et al., Hepatology. 2005, 41(6):1313-1321, and the ISHAK fibrosis scoring system (see Ishak et al., J Hepatol. 1995; 22:696-9), the contents of each of which are incorporated by reference herein in their entireties.

In some embodiments, the severity of fibrosis is determined prior to administration of the combination of (a) the compound of Formula (I), or a pharmaceutically acceptable salt thereof, and (b) an ASK1 inhibitor, or a pharmaceutically acceptable salt thereof. In some embodiments, the severity of fibrosis is determined prior to administration of the combination of (a) the compound of Formula (I), or a pharmaceutically acceptable salt thereof, and (b) an ASK1 inhibitor, or a pharmaceutically acceptable salt thereof. In some embodiments, the severity of fibrosis is determined during the period of time or after the period of time of administration of the combination of (a) and (b).

In some embodiments, the severity of flibrosis is determined prior to administration of the combination of (a) the compound of Formula (I), or a pharmaceutically acceptable salt thereof, and (b) an ACC inhibitor, or a pharmaceutically acceptable salt thereof. In some embodiments, the severity of fibrosis is determined prior to administration of the combination of (a) the compound of Formula (I), or a pharmaceutically acceptable salt thereof, and (b) an ACC inhibitor, or a pharmaceutically acceptable salt thereof.

In some embodiments, the severity of flibrosis is determined prior to administration of the combination of (a) the compound of Formula (I), or a pharmaceutically acceptable salt thereof, and (b) a caspase inhibitor, or a pharmaceutically acceptable salt thereof. In some embodiments, the severity of flibrosis is determined prior to administration of the combination of (a) the compound of Formula (I), or a pharmaceutically acceptable salt thereof, and (b) a caspase inhibitor, or a pharmaceutically acceptable salt thereof.

In some embodiments, the severity of flibrosis is determined prior to administration of the combination of (a) the compound of Formula (I), or a pharmaceutically acceptable salt thereof, and (b) MCR antagonist, or a pharmaceutically acceptable salt thereof. In some embodiments, the severity of fibrosis is determined prior to administration of the combination of (a) the compound of Formula (I), or a pharmaceutically acceptable salt thereof, and (b) a MCR antagonist, or a pharmaceutically acceptable salt thereof.

In some embodiments, the severity of flibrosis is determined prior to administration of the combination of (a) the compound of Formula (I), or a pharmaceutically acceptable salt thereof, and (b) a statin, or a pharmaceutically acceptable salt thereof. In some embodiments, the severity of flibrosis is determined prior to administration of the combination of (a) the compound of Formula (I), or a pharmaceutically acceptable salt thereof, and (b) a statin, or a pharmaceutically acceptable salt thereof.

In some embodiments, the severity of flibrosis is determined prior to administration of the combination of (a) the compound of Formula (I), or a pharmaceutically acceptable salt thereof, and (b) a PCSK9 inhibitor, or a pharmaceutically acceptable salt thereof. In some embodiments, the severity of flibrosis is determined prior to administration of the combination of (a) the compound of Formula (I), or a pharmaceutically acceptable salt thereof, and (b) a PCSK9 inhibitor, or a pharmaceutically acceptable salt thereof.

In some embodiments, a decrease in the severity of fibrosis during the period of time or after the period of time of administration of the combination of (a) and (b) compared to prior to administration of the combination of (a) and (b) indicates treatment of NAFLD. In some embodiments, a decrease in the severity of fibrosis, a lack of further progression of fibrosis and/or cirrhosis, or a slowing of the progression of fibrosis and/or cirrhosis indicates treatment of NAFLD. In some embodiments, the severity of fibrosis is determined using a scoring system such as any of the fibrosis scoring systems described herein, for example, the score can indicate the stage of fibrosis, e.g., stage 0 (no fibrosis), stage 1, stage 2, stage 3, and stage 4 (cirrhosis) (see, e.g., Kleiner et al). In some embodiments, a decrease in the stage of the fibrosis is a decrease in the severity of the fibrosis. For example, a decrease by 1, 2, 3, or 4 stages is a decrease in the severity of the fibrosis. In some embodiments, a decrease in the stage, e.g., from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 indicates treatment of NAFLD. In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 following administration of the combination of (a) and (b) compared to prior to administration of the combination of (a) and (b). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 during the period of time of administration of the combination of (a) and (b) compared to prior to administration of the combination of (a) and (b). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 after the period of time of administration of the combination of (a) and (b) compared to prior to administration of the combination of (a) and (b).

In some embodiments, the presence of NAFLD is determined by one or more biomarkers indicative of one or more of liver damage, inflammation, liver fibrosis, and/or liver cirrhosis or scoring systems thereof. In some embodiments, the severity of NAFLD is determined by one or more biomarkers indicative of one or more of liver damage, inflammation, liver fibrosis, and/or liver cirrhosis or scoring systems thereof. The level of the biomarker can be determined by, for example, measuring, quantifying, and monitoring the expression level of the gene or mRNA encoding the biomarker and/or the peptide or protein of the biomarker. Non-limiting examples of biomarkers indicative of one or more of liver damage, inflammation, liver fibrosis, and/or liver cirrhosis and/or scoring systems thereof include the aspartate aminotransferase (AST) to platelet ratio index (APRI); the aspartate aminotransferase (AST) and alanine aminotransferase (ALT) ratio (AAR); the FIB-4 score, which is based on the APRI, alanine aminotransferase (ALT) levels, and age of the subject (see, e.g., McPherson et al., Gut. 2010 September; 59(9):1265-9, which is incorporated by reference herein in its entirety); hyaluronic acid; pro-inflammatory cytokines; a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score, see, e.g., Lichtinghagen R, et al., J Hepatol. 2013 August; 59(2):236-42, which is incorporated by reference herein in its entirety). In some embodiments, the presence of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, the level of aspartate aminotransferase (AST) does not increase. In some embodiments, the level of aspartate aminotransferase (AST) decreases. In some embodiments, the level of alanine aminotransferase (ALT) does not increase. In some embodiments, the level of alanine aminotransferase (ALT) decreases. In some embodiments, the “level” of an enzyme refers to the concentration of the enzyme, e.g., within blood. For example, the level of AST or ALT can be expressed as Units/L.

In some embodiments, the severity of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73, which is incorporated by reference herein in its entirety), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, hepatic inflammation is determined by the level of liver inflammation biomarkers, e.g., pro-inflammatory cytokines. Non-limiting examples of biomarkers indicative of liver inflammation include interleukin-(IL) 6, interleukin-(IL) 1β, tumor necrosis factor (TNF)-α, transforming growth factor (TGF)-β, monocyte chemotactic protein (MCP)-1, C-reactive protein (CRP), PAI-1, and collagen isoforms such as Col1a1, Col1a2, and Col4a1 (see, e.g., Neuman, et al., Can J Gastroenterol Hepatol. 2014 December; 28(11): 607-618 and U.S. Pat. No. 9,872,844, each of which are incorporated by reference herein in their entireties). Liver inflammation can also be assessed by change of macrophage infiltration, e.g., measuring a change of CD68 expression level. In some embodiments, liver inflammation can be determined by measuring or monitoring serum levels or circulating levels of one or more of interleukin-(IL) 6, interleukin-(IL) 1β, tumor necrosis factor (TNF)-α, transforming growth factor (TGF)-β, monocyte chemotactic protein (MCP)-1, and C-reactive protein (CRP).

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) an ASK1 inhibitor, or a pharmaceutically acceptable salt thereof.

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) an ACC inhibitor, or a pharmaceutically acceptable salt thereof.

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) a caspase inhibitor, or a pharmaceutically acceptable salt thereof.

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) MCR antagonist, or a pharmaceutically acceptable salt thereof.

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) a statin, or a pharmaceutically acceptable salt thereof.

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) a PCSK9 inhibitor, or a pharmaceutically acceptable salt thereof.

In some embodiments, the level of one or more biomarkers indicative of one or more of liver damage, inflammation, liver fibrosis, and/or liver cirrhosis is determined during the period of time or after the period of time of administration of the combination of (a) and (b). In some embodiments, a decrease in the level of one or more biomarkers indicative of one or more of liver damage, inflammation, liver fibrosis, and/or liver cirrhosis during the period of time or after the period of time of administration of the combination of (a) and (b) compared to prior to administration of the combination of (a) and (b) indicates treatment of NAFLD. For example, a decrease in the level of one or more biomarkers indicative of one or more of liver damage, inflammation, liver fibrosis, and/or liver cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% indicates treatment of NAFLD. In some embodiments, the decrease in the level of one or more biomarkers indicative of one or more of liver damage, inflammation, liver fibrosis, and/or liver cirrhosis following administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis during the period of time of administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis after the period of time of administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the level of serum bile acids is determined by, for example, an ELISA enzymatic assay or the assays for the measurement of total bile acids as described in Danese et al., PLoS One. 2017; 12(6): e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of (a) and (b). In some embodiments, the NAFLD is NAFLD with attendant cholestasis. In cholestasis, the release of bile, including bile acids, from the liver is blocked. Bile acids can cause hepatocyte damage (see, e.g., Perez M J, Briz O. World J Gastroenterol. 2009 Apr. 14; 15(14):1677-89) likely leading to or increasing the progression of fibrosis (e.g., cirrhosis) and increasing the risk of hepatocellular carcinoma (see, e.g., Sorrentino P et al. Dig Dis Sci. 2005 June; 50(6):1130-5 and Satapathy S K and Sanyal A J. Semin Liver Dis. 2015, 35(3):221-35, each of which are incorporated by reference herein in their entireties). In some embodiments, the NAFLD with attendant cholestasis is NASH with attendant cholestasis. In some embodiments, the treatment of NAFLD comprises treatment of pruritus. In some embodiments, the treatment of NAFLD with attendant cholestasis comprises treatment of pruritus. In some embodiments, a subject with NAFLD with attendant cholestasis has pruritus.

In some embodiments, treatment of NAFLD comprises an increase in adiponectin. It is thought that the compound of Formula (I) may be a selective activator of a highly limited number of PPARγ pathways including pathways regulated by adiponectin. Adiponectin is an anti-fibrotic and anti-inflammatory adipokine in the liver (see e.g., Park et al., Curr Pathobiol Rep. 2015 Dec. 1; 3(4): 243-252.). In some embodiments, the level of adiponectin is determined by, for example, an ELISA enzymatic assay. In some embodiments, the adiponectin level in the subject is increased by at least about 30%, at least about 68%, at least about 175%, or at least about 200%. In some embodiments, the increase is by at least about 175%.

In some embodiments, the level of adiponectin is determined for a sample from the subject prior to administration of the combination of (a) the compound of Formula (I), or a pharmaceutically acceptable salt thereof, and (b) an ASK1 inhibitor, or a pharmaceutically acceptable salt thereof.

In some embodiments, the level of adiponectin is determined for a sample from the subject prior to administration of the combination of (a) the compound of Formula (I), or a pharmaceutically acceptable salt thereof, and (b) an ACC inhibitor, or a pharmaceutically acceptable salt thereof.

In some embodiments, the level of adiponectin is determined for a sample from the subject prior to administration of the combination of (a) the compound of Formula (I), or a pharmaceutically acceptable salt thereof, and (b) a caspase inhibitor, or a pharmaceutically acceptable salt thereof.

In some embodiments, the level of adiponectin is determined for a sample from the subject prior to administration of the combination of (a) the compound of Formula (I), or a pharmaceutically acceptable salt thereof, and (b) MCR antagonist, or a pharmaceutically acceptable salt thereof.

In some embodiments, the level of adiponectin is determined for a sample from the subject prior to administration of the combination of (a) the compound of Formula (I), or a pharmaceutically acceptable salt thereof, and (b) a statin, or a pharmaceutically acceptable salt thereof.

In some embodiments, the level of adiponectin is determined for a sample from the subject prior to administration of the combination of (a) the compound of Formula (I), or a pharmaceutically acceptable salt thereof, and (b) a PCSK9 inhibitor, or a pharmaceutically acceptable salt thereof.

In some embodiments, the level of adiponectin is determined for a sample from the subject prior to administration of the combination of (a) the compound of Formula (I), or a pharmaceutically acceptable salt thereof, and (b) an ASK1 inhibitor, or a pharmaceutically acceptable salt thereof. In some embodiments, the level of adiponectin is determined during the period of time or after the period of time of administration of the combination of (a) and (b). In some embodiments, an increase in the level of adiponectin during the period of time or after the period of time of administration of the combination of (a) and (b) compared to prior to administration of the combination of (a) and (b) indicates treatment of NAFLD. For example, an increase in the level of adiponectin by at least about 30%, at least about 68%, at least about 175%, or at least about 200% indicates treatment of NAFLD. In some embodiments, the increase in the level of adiponectin following administration of the combination of (a) and (b) is at least about 200%.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) an ASK1 inhibitor, or a pharmaceutically acceptable salt thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) an ASK1 inhibitor, or a pharmaceutically acceptable salt thereof, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) an ASK1 inhibitor, or a pharmaceutically acceptable salt thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) an ASK1 inhibitor, or a pharmaceutically acceptable salt thereof, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) an ASK1 inhibitor, or a pharmaceutically acceptable salt thereof, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) an ASK1 inhibitor, or a pharmaceutically acceptable salt thereof, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) an ASK1 inhibitor, or a pharmaceutically acceptable salt thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) an ASK1 inhibitor, or a pharmaceutically acceptable salt thereof, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of an ASK1 inhibitor, or a pharmaceutically acceptable salt thereof, or a pharmaceutically acceptable salt thereof. In some embodiments, the amounts of (a) and (b) together are effective in treating NAFLD.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) an ACC inhibitor, or a pharmaceutically acceptable salt thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) an ACC inhibitor, or a pharmaceutically acceptable salt thereof, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) an ACC inhibitor, or a pharmaceutically acceptable salt thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) an ACC inhibitor, or a pharmaceutically acceptable salt thereof, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) an ACC inhibitor, or a pharmaceutically acceptable salt thereof, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) an ACC inhibitor, or a pharmaceutically acceptable salt thereof, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) an ACC inhibitor, or a pharmaceutically acceptable salt thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) an ACC inhibitor, or a pharmaceutically acceptable salt thereof, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of an ACC inhibitor, or a pharmaceutically acceptable salt thereof, or a pharmaceutically acceptable salt thereof. In some embodiments, the amounts of (a) and (b) together are effective in treating NAFLD.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) a caspase inhibitor, or a pharmaceutically acceptable salt thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) a caspase inhibitor, or a pharmaceutically acceptable salt thereof, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) a caspase inhibitor, or a pharmaceutically acceptable salt thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) a caspase inhibitor, or a pharmaceutically acceptable salt thereof, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) a caspase inhibitor, or a pharmaceutically acceptable salt thereof, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) a caspase inhibitor, or a pharmaceutically acceptable salt thereof, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) a caspase inhibitor, or a pharmaceutically acceptable salt thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) a caspase inhibitor, or a pharmaceutically acceptable salt thereof, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of a caspase inhibitor, or a pharmaceutically acceptable salt thereof, or a pharmaceutically acceptable salt thereof. In some embodiments, the amounts of (a) and (b) together are effective in treating NAFLD.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) an MCR antagonist, or a pharmaceutically acceptable salt thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) an MCR antagonist, or a pharmaceutically acceptable salt thereof, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) an MCR antagonist, or a pharmaceutically acceptable salt thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) an MCR antagonist, or a pharmaceutically acceptable salt thereof, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) an MCR antagonist, or a pharmaceutically acceptable salt thereof, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) an MCR antagonist, or a pharmaceutically acceptable salt thereof, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) an MCR antagonist, or a pharmaceutically acceptable salt thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) an MCR antagonist, or a pharmaceutically acceptable salt thereof, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of an MCR antagonist, or a pharmaceutically acceptable salt thereof, or a pharmaceutically acceptable salt thereof. In some embodiments, the amounts of (a) and (b) together are effective in treating NAFLD.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) a statin, or a pharmaceutically acceptable salt thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) a statin, or a pharmaceutically acceptable salt thereof, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) a statin, or a pharmaceutically acceptable salt thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) a statin, or a pharmaceutically acceptable salt thereof, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) a statin, or a pharmaceutically acceptable salt thereof, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) a statin, or a pharmaceutically acceptable salt thereof, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) a statin, or a pharmaceutically acceptable salt thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) a statin, or a pharmaceutically acceptable salt thereof, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of a statin, or a pharmaceutically acceptable salt thereof, or a pharmaceutically acceptable salt thereof. In some embodiments, the amounts of (a) and (b) together are effective in treating NAFLD.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) a PCSK9 inhibitor, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) a PCSK9 inhibitor, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) a PCSK9 inhibitor, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) a PCSK9 inhibitor, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) a PCSK9 inhibitor, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) a PCSK9 inhibitor, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) a PCSK9 inhibitor, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) a PCSK9 inhibitor, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of a PCSK9 inhibitor. In some embodiments, the amounts of (a) and (b) together are effective in treating NAFLD.

In some embodiments, the ACC inhibitor, or a pharmaceutically acceptable salt thereof, is selected from the group consisting of:

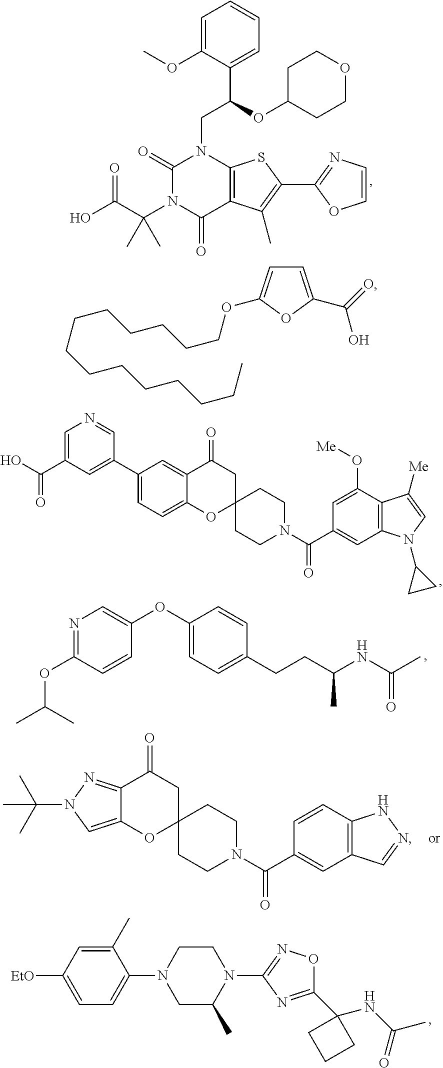

or a pharmaceutically acceptable salt thereof. In some embodiments, the ACC inhibitor is

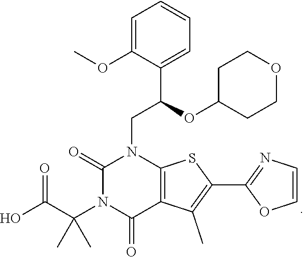 [SMILES: COc1ccccc1[C@H](Cn1c(=O)n(C(C)(C)C(=O)O)c(=O)c2c(C)c(-c3ncco3)sc21)OC1CCOCC1]

In some embodiments, the amount of the compound of Formula (I), or a pharmaceutically acceptable salt thereof, is from about 1 to about 350 mg, or any value in between. For example, about 1 to about 175 mg, about 175 to about 350 mg, about 90 to about 260 mg, or about 150 to 200 mg.

In some embodiments, the amount of the ACC inhibitor, or a pharmaceutically acceptable salt thereof, is from about 1 to about 400 mg, or any value in between. For example, about 1 to about 150 mg, about 50 to about 250 mg, about 100 to about 300 mg, or about 150 to about 400 mg.

In some embodiments, the ACC inhibitor, or a pharmaceutically acceptable salt thereof, is administered to the subject twice a day, daily, every other day, three times a week, twice a week, weekly, every other week, twice a month, or monthly. In some embodiments, the ACC inhibitor, or a pharmaceutically acceptable salt thereof, is administered to the subject daily.

In some embodiments, the caspase inhibitor, or a pharmaceutically acceptable salt thereof, is selected from the group consisting of:

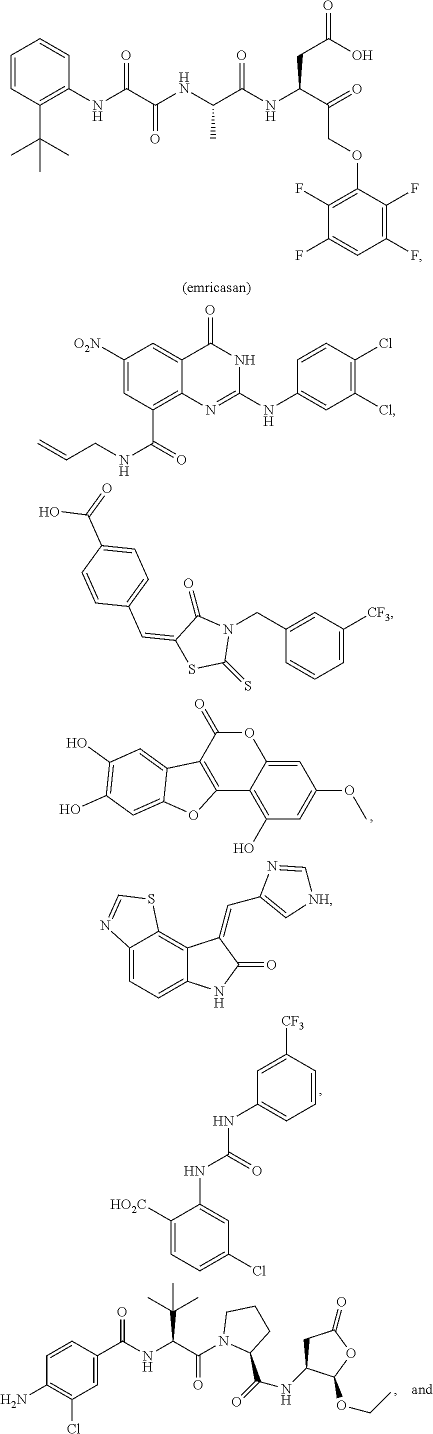

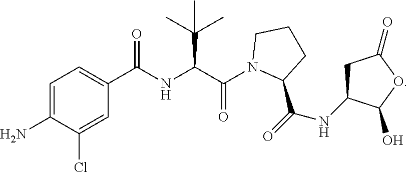 [SMILES: CC(C)(C)[C@H](NC(=O)c1ccc(N)c(Cl)c1)C(=O)N1CCC[C@H]1C(=O)N[C@H]1CC(=O)O[C@H]1O]

a pharmaceutically acceptable salt thereof. In some embodiments, the caspase inhibitor is emricasan.

In some embodiments, the amount of the compound of Formula (I), or a pharmaceutically acceptable salt thereof, is from about 1 to about 350 mg, or any value in between. For example, about 1 to about 175 mg, about 175 to about 350 mg, about 90 to about 260 mg, or about 150 to 200 mg.

In some embodiments, the amount of the caspase inhibitor, or a pharmaceutically acceptable salt thereof, is from about 1 to about 500 mg, or any value in between. For example, about 1 to about 10 mg, about 5 to about 25 mg, about 20 to about 100 mg, about 50 to about 200 mg, about 100 to about 300 mg, about 200 to about 350 mg, about 250 to about 400 mg, about 300 to about 450 mg, or about 350 to about 500 mg.

In some embodiments, the caspase inhibitor, or a pharmaceutically acceptable salt thereof, is administered to the subject twice a day, daily, every other day, three times a week, twice a week, weekly, every other week, twice a month, or monthly. In some embodiments, the caspase inhibitor, or a pharmaceutically acceptable salt thereof, is administered to the subject daily.

In some embodiments, the MCR antagonist, or a pharmaceutically acceptable salt thereof, is selected from the group consisting of: spironolactone, finerenone, eplerenone, canrenone, mexrenone, amlodipine, apararenone, benidipine, esaxerenone, felodipine, nefedipine, nimodipine, and nitrendipine, or a pharmaceutically acceptable salt thereof. In some embodiments, the MCR antagonist is spironolactone.

In some embodiments, the amount of the compound of Formula (I), or a pharmaceutically acceptable salt thereof, is from about 0.5 to about 150 mg, or any value in between. For example, about 0.5 to about 5 mg, about 1 to about 10 mg, about 8 to about 20 mg, about 15 to about 30 mg, about 20 to about 40 mg, about 25 to about 50 mg, about 40 to about 75 mg, about 60 to about 90 mg, about 75 to about 100 mg, about 80 to about 125 mg, about 120 to about 140 mg, or about 130 to about 150 mg.

In some embodiments, the MCR antagonist is spironolactone. In some embodiments, about 10 to 100 mg of spironolactone is administered, or any value in between. For example, 10 mg, 25 mg, 50 mg, 75 mg, or 100 mg. In some embodiments, the MCR antagonist is eplerenone. In some embodiments, about 10 to 100 mg of eplerenone is administered, or any value in between. For example, 10 mg, 25 mg, 50 mg, 75 mg, or 100 mg. In some embodiments, the MCR antagonist is canrenone. In some embodiments, about 10 to 100 mg canrenone is administered, or any value in between. For example, 10 mg, 25 mg, 50 mg, 75 mg, or 100 mg.

In some embodiments, the MCR antagonist, or a pharmaceutically acceptable salt thereof, is administered to the subject twice a day, daily, every other day, three times a week, twice a week, weekly, every other week, twice a month, or monthly. In some embodiments, the MCR antagonist, or a pharmaceutically acceptable salt thereof, is administered to the subject daily.

In some embodiments, the statin, or a pharmaceutically acceptable salt thereof, is selected from the group consisting of: atorvastatin, cerivastatin, fluvastatin, lovastatin, mevastatin, pitavastatin, pravastatin, rosuvastatin, or simvastatin, or a pharmaceutically acceptable salt thereof. In some embodiments, the statin is atorvastatin.

In some embodiments, the amount of the compound of Formula (I), or a pharmaceutically acceptable salt thereof, is from about 1 to about 350 mg, or any value in between. For example, about 1 to about 175 mg, about 175 to about 350 mg, about 90 to about 260 mg, or about 150 to 200 mg.

In some embodiments, the amount of the statin, or a pharmaceutically acceptable salt thereof, is from about 1 to about 80 mg, or any value in between. For example, about 1 to about 20 mg, about 5 to about 30 mg, about 10 to about 40 mg, about 15 to about 50 mg, about 20 to about 60 mg, about 30 to about 70 mg, or about 40 to about 80 mg.

In some embodiments, the statin is atorvastatin. In some embodiments, about 5 to 80 mg of atorvastatin is administered, or any value in between. For example, 5 mg, 10 mg, 20 mg, 40 mg, 60 mg, or 80 mg. In some embodiments, the statin is fluvastatin. In some embodiments, about 10 to 80 mg of fluvastatin is administered, or any value in between For example, 10 mg, 20 mg, 30 mg, 40 mg, 50 mg, 60 mg, or 80 mg. In some embodiments, the statin is lovastatin. In some embodiments, about 5 to 80 mg of lovastatin is administered, or any value in between. For example, 5 mg, 10 mg, 20 mg, 40 mg, 60 mg, or 80 mg. In some embodiments, the statin is rosuvastatin. In some embodiments, about 1 to 40 mg rosuvastatin is administered, or any value in between. For example, 1 mg, 5 mg, 10 mg, 15 mg, 20 mg, 25 mg, 30 mg, 35 mg, or 40 mg. In some embodiments, the statin is simvastatin. In some embodiments, about 1 to 40 mg of simvastatin is administered, or any value in between. For example, 1 mg, 5 mg, 10 mg, 15 mg, 20 mg, 25 mg, 30 mg, 35 mg, or 40 mg.

In some embodiments, the statin, or a pharmaceutically acceptable salt thereof, is administered to the subject twice a day, daily, every other day, three times a week, twice a week, weekly, every other week, twice a month, or monthly. In some embodiments, the statin, or a pharmaceutically acceptable salt thereof, is administered to the subject daily.

In some embodiments, the PCSK9 inhibitor is selected from the group consisting of: alirocumab, evolocumab, bococizumab, 1D05-IgG2, RG-7652, LY3015014, and inclisiran. In some embodiments, the PCSK9 inhibitor is alirocumab.

In some embodiments, the amount of the compound of Formula (I), or a pharmaceutically acceptable salt thereof, is from about 1 to about 350 mg, or any value in between. For example, about 1 to about 175 mg, about 175 to about 350 mg, about 90 to about 260 mg, or about 150 to 200 mg.

In some embodiments, the total amount of the PCSK9 inhibitor administered per month is from about 50 to about 450 mg, or any value in between. For example, about 25 to about 50 mg, about 40 to about 65 mg, about 55 to about 80 mg, about 70 to about 95 mg, about 85 to about 110 mg, about 90 to about 125 mg, about 95 to about 140 mg, about 100 to about 155 mg, about 125 to about 175 mg, about 150 to about 200 mg, or about 175 to about 225 mg every two weeks (twice per month); or about 150 to about 200 mg, about 175 to about 225 mg, about 200 to about 250 mg, about 225 to about 275 mg, about 250 to about 300 mg, about 275 to about 325 mg, about 300 to about 350 mg, about 325 to about 375 mg, about 350 to about 400 mg, about 375 to about 425 mg, or about 400 to about 450 mg once per month.

In some embodiments, the PCSK9 inhibitor is administered to the subject daily, every other day, three times a week, twice a week, weekly, every other week, twice a month, or monthly. In some embodiments, the PCSK9 inhibitor is administered to the subject twice a month, or monthly.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the amount of the compound of Formula (I), or a pharmaceutically acceptable salt thereof, is from about 0.1 to about 15 milligrams (mg), or any value in between. For example, from about 0.1 to about 10 mg, about 5 to about 15 mg, or about 2 to about 12 mg. In some embodiments, the compound of Formula (I), or a pharmaceutically acceptable salt thereof, is administered at a dose of about 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, or 15.0 mg. In some embodiments, the dose is a therapeutically effective amount.

In some embodiments, the compound of Formula (I), or a pharmaceutically acceptable salt thereof, is administered to the subject twice a day, daily, every other day, three times a week, twice a week, weekly, every other week, twice a month, or monthly. In some embodiments, the compound of Formula (I), or a pharmaceutically acceptable salt thereof, is administered to the subject daily.

In some embodiments, the compound of Formula (I), or a pharmaceutically acceptable salt thereof, is administered to the subject daily and at a dose of about 3 mg. In some embodiments, the compound of Formula (I), or a pharmaceutically acceptable salt thereof, is administered at a dose from about 0.1 to about 10.0 mg per day. In some embodiments, the compound of Formula (I), or a pharmaceutically acceptable salt thereof, is administered at a dose from about 0.1 to about 3 mg per day. In some embodiments, the compound of Formula (I), or a pharmaceutically acceptable salt thereof, is administered at a dose about 0.5 mg per day. In some embodiments, the compound of Formula (I), or a pharmaceutically acceptable salt thereof, is administered at a dose about 1 mg per day. In some embodiments, the compound of Formula (I), or a pharmaceutically acceptable salt thereof, is administered at a dose about 2 mg per day.

In some of any of the above embodiments, the compound of Formula (I) is in the form of a besylate salt. In some embodiments, the compound of Formula (I) is in the form of an HCl salt. In some embodiments, the compound of Formula (I) is in the form of an HBr salt. In some embodiments, the compound of Formula (I) is in the form of a tosylate salt.

In some embodiments, the ASK1 inhibitor, or a pharmaceutically acceptable salt thereof, is selected from the group consisting of: selonsertib, MSC 2032964A, NQDI 1, TC ASK 10, AGI-1067, GS-444217, K811, K812, or SRT-015, or a pharmaceutically acceptable salt thereof. In some embodiments, the ASK1 inhibitor is selonsertib.

In some embodiments, the amount of the compound of Formula (I), or a pharmaceutically acceptable salt thereof, is from about 1 to about 350 mg, or any value in between. For example, about 1 to about 175 mg, about 175 to about 350 mg, about 90 to about 260 mg, or about 150 to 200 mg.

In some embodiments, the amount of the ASK1 inhibitor, or a pharmaceutically acceptable salt thereof, is from about 1 to about 25 mg, or any value in between. For example, about 1 to about 5 mg, about 5 to about 10 mg, about 10 to about 15 mg, about 15 to about 20 mg, or about 20 to about 25 mg.

In some embodiments, the ASK1 inhibitor, or a pharmaceutically acceptable salt thereof, is administered to the subject twice a day, daily, every other day, three times a week, twice a week, weekly, every other week, twice a month, or monthly. In some embodiments, the ASK1 inhibitor, or a pharmaceutically acceptable salt thereof, is administered to the subject daily.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic.

In some embodiments, the treatment of NAFLD, e.g., NAFL or NASH, comprises a reduction in hepatic steatosis. For example, hepatic steatosis is decreased by at least 2%, 3%, 4%, 5%, 6%, 7%, 8%. 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more than 99% following administration of (a) and (b) for a period of time.

In some embodiments, the treatment of NAFLD, e.g., NAFL or NASH, is assessed using the NAFLD Activity Score (NAS). In some embodiments, treatment of NAFLD comprises a decrease in the NAS. In some embodiments, the NAS for a sample from the subject following administration is 7 or less. In some embodiments, the NAS for a sample from the subject following administration is 5 or less, 4 or less, 3 or less, or 2 or less. In some embodiments, the NAFLD activity score (NAS) for a sample from the subject following administration during the period of time is 7 or less. In some embodiments, the NAS for a sample from the subject following administration during the period of time is 5 or less, 4 or less, 3 or less, or 2 or less. In some embodiments, the sample from the subject is from a liver biopsy.

In some embodiments, the treatment of NAFLD, e.g., NAFL or NASH, can be assessed using the NAFLD Activity Score (NAS). In some embodiments, the NAS for a sample from the subject following administration is reduced by 1 or more, 2 or more, 3 or more, 4 or more, 5 or more, or 6 or more. In some embodiments, the NAS for a sample from the subject following administration is reduced by 1, 2, 3, 4, 5, or 6. In some embodiments, the NAFLD activity score (NAS) for a sample from the subject following administration during the period of time is reduced by 1 or more, 2 or more, 3 or more, 4 or more, 5 or more, or 6 or more. In some embodiments, the NAS for a sample from the subject following administration during the period of time is reduced by 1, 2, 3, 4, 5, or 6. In some embodiments, the sample from the subject is from a liver biopsy.

In some embodiments, the treatment of NAFLD, e.g., NAFL or NASH, comprises treatment of hepatic inflammation. In some embodiments, the severity of the hepatic inflammation is decreased by about 1% to about 50%, about 25% to about 75%, or about 50% to about 100%. In some embodiments, the severity of hepatic inflammation is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the treatment of NAFLD, e.g., NAFL or NASH, comprises treatment of fibrosis. In some embodiments, the treatment of the NAFLD comprises treatment of cirrhosis (e.g., stage 4 of fibrosis). In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

In some embodiments, the adiponectin level in the subject is increased by at least about 30%, at least about 68%, at least about 175%, or at least about 200%. In some embodiments, the increase is by at least about 175%.

In some embodiments, the level of aspartate aminotransferase (AST) in the subject does not increase. In some embodiments, the level of aspartate aminotransferase (AST) in the subject decreases. In some embodiments, the level of alanine aminotransferase (ALT) in the subject does not increase. In some embodiments, the level of alanine aminotransferase (ALT) in the subject decreases. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, a non-invasive liver fibrosis marker does not increase or decreases. In some embodiments, the non-invasive liver fibrosis marker is Enhanced Liver Fibrosis (ELF) panel.

In some embodiments, treatment of NAFLD comprises a decrease in the level of one or more biomarkers indicative of one or more of liver damage, inflammation, fibrosis, and/or cirrhosis, e.g., any of the biomarkers as described herein. In some embodiments, treatment of NAFLD comprises a decrease in the level of one or more biomarkers indicative of one or more of liver damage, inflammation, fibrosis, and/or cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the treatment of NAFLD comprises treatment of pruritus.

In some embodiments, the subject has liver fibrosis associated with the NAFLD. In some embodiments, the subject has hepatic cirrhosis (e.g., stage 4 fibrosis) associated with the NAFLD. In some embodiments, the subject has liver fibrosis as a comorbidity. In some embodiments, the subject has hepatic cirrhosis (e.g., stage 4 fibrosis) as a comorbidity. In some embodiments, the subject has liver fibrosis caused by the NAFLD. In some embodiments, the subject has hepatic cirrhosis (e.g., stage 4 fibrosis) caused by the NAFLD.

In some embodiments, the NAFLD is simple nonalcoholic fatty liver (NAFL). In some embodiments, the NAFLD is NAFL with attendant liver fibrosis. In some embodiments, the NAFLD is NAFL with attendant liver cirrhosis.

In some embodiments, the NAFLD is nonalcoholic steatohepatitis (NASH). In some embodiments, the NAFLD is NASH with attendant liver fibrosis. In some embodiments, the NAFLD is NASH with attendant liver cirrhosis.

In some embodiments, the method further comprises performing a liver biopsy to determine the NAFLD activity score of the biopsy sample obtained from the subject.

In some embodiments, (a) and (b) are administered prophylactically.

In some embodiments, the subject was previously treated, before the period of time, with one or more therapeutic agents, e.g., treatment with at least one NAFLD treatment, NASH treatment, type 2 diabetes treatment, obesity treatment, metabolic syndrome treatment, liver disease treatment, cardiovascular treatment, heart failure treatment, hypertension treatment. In some embodiments, the one or more therapeutic agents that were administered to the patient before the period of time was unsuccessful (e.g., therapeutically unsuccessful as determined by a physician). In some embodiments, the unsuccessful treatment did not comprises or consist essentially of administration of (a) and (b).

The following exemplary embodiments are also provided herein:
1) A method of treating non-alcoholic fatty liver disease (NAFLD) in a subject in need thereof comprising administering to the subject

(a) the compound of Formula (I), or a pharmaceutically acceptable salt thereof, and

(b) an ACC inhibitor, or a pharmaceutically acceptable salt thereof,

wherein the amounts of (a) and (b) together are effective in treating NAFLD.

2) A method of treating a subject, the method comprising:

selecting a subject having non-alcoholic fatty liver disease (NAFLD); and

administering

(a) the compound of Formula (I), or a pharmaceutically acceptable salt thereof,

(b) an ACC inhibitor, or a pharmaceutically acceptable salt thereof, to the selected subject, wherein the amounts of (a) and (b) together are effective in treating NAFLD.

3) A method of treating a subject, the method comprising:

identifying a subject having non-alcoholic fatty liver disease (NAFLD); and administering

(a) the compound of Formula (I), or a pharmaceutically acceptable salt thereof, and

(b) an ACC inhibitor, or a pharmaceutically acceptable salt thereof, to the selected subject, wherein the amounts of (a) and (b), together are effective in treating NAFLD.

4) The method of any one of embodiments 1 to 3, wherein the compound of Formula I is

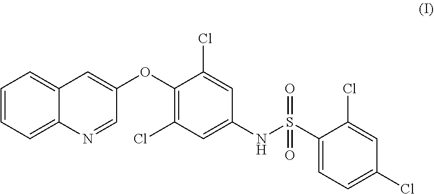 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
5) The method of any one of embodiments 1 to 4 wherein the ACC inhibitor, or a pharmaceutically acceptable salt thereof, is a dual ACC inhibitor, or a pharmaceutically acceptable salt thereof.
6) The method of any one of embodiments 1 to 5, wherein the treatment of NAFLD comprises a reduction in hepatic steatosis.
7) The method of any one of embodiments 1 to 6, wherein the treatment of NAFLD comprises a reduction in hepatic inflammation.
8) The method of any one of embodiments 1 to 7, wherein the NAFLD activity score (NAS) following administration is 7 or less.
9) The method of any one of embodiments 1 to 8, wherein the NAS is 5 or less.
10) The method of any one of embodiments 1 to 9, wherein the NAS is 3 or less.
11) The method of any one of embodiments 1 to 10, wherein the subject has hepatic cirrhosis associated with the NAFLD.
12) The method of any one of embodiments 1 to 11, wherein the subject has hepatic cirrhosis as a comorbidity.
13) The method of any one of embodiments 1 to 12, wherein the subject has hepatic cirrhosis caused by the NAFLD.
14) The method of any one of embodiments 1 to 13, wherein the NAFLD is NAFL with attendant liver cirrhosis.
15) The method of any one of embodiments 1 to 14, wherein the treatment of the NAFLD comprises treatment of liver cirrhosis.
16) The method of any one of embodiments 1 to 15, wherein the treatment of NAFLD decreases the level of serum bile acids in the subject.
17) The method of any one of embodiments 1 to 16, wherein the treatment of NAFLD comprises treatment of pruritus.
18) The method of any one of embodiments 1 to 10, wherein the NAFLD is simple nonalcoholic fatty liver (NAFL).
19) The method of embodiment 18, wherein the treatment of NAFL comprises treatment of pruritus.
20) The method of embodiment 18, wherein the treatment of NAFL decreases the level of serum bile acids in the subject.
21) The method of any one of embodiments 1 to 13 or 15 to 17, wherein the NAFLD is nonalcoholic steatohepatitis (NASH).

22) The method of any one of embodiments 1 to 13, 15 to 17, or 21, wherein the NAFLD is NASH with attendant liver cirrhosis.

23) The method of any one of embodiments 1 to 13, 15 to 17, or 21 to 22, wherein the treatment of NASH decreases the level of serum bile acids in the subject.
24) The method of any one of embodiments 1 to 13, 15 to 17, or 21 to 23, wherein the treatment of NASH comprises treatment of pruritus.
25) A method of treating fibrosis in a subject in need thereof comprising administering to the subject

(a) the compound of Formula (I), or a pharmaceutically acceptable salt thereof, and

(b) an ACC inhibitor, or a pharmaceutically acceptable salt thereof,

wherein the amounts of (a) and (b) together are effective in treating NAFLD.

26) The method of embodiment 25, wherein the compound of Formula I is

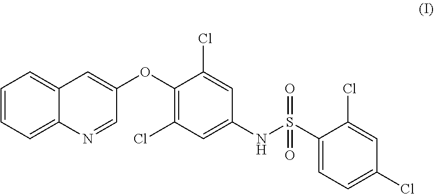 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
27) The method of embodiment 25 or 26, wherein the ACC inhibitor, or a pharmaceutically acceptable salt thereof, is

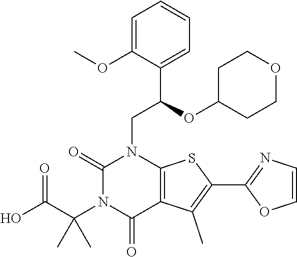 [SMILES: COc1ccccc1[C@H](Cn1c(=O)n(C(C)(C)C(=O)O)c(=O)c2c(C)c(-c3ncco3)sc21)OC1CCOCC1]

or a pharmaceutically acceptable salt thereof.
28) The method of any one of embodiments 25 to 27, wherein the fibrosis is cirrhosis.
29) The method of any one of embodiments 25 to 28, wherein the fibrosis is associated with NAFLD.
30) The method of any one of embodiments 25 to 29, wherein the fibrosis is caused by NAFLD.
31) The method of any one of embodiments 25 to 30, wherein the NAFLD is NASH.
32) The method of any one of embodiments 25 to 31, wherein the treatment of fibrosis comprises a decrease in the stage of fibrosis, a lack of progression of the fibrosis, or a slowing in the progression of the fibrosis.
33) The method of any one of embodiments 25 to 32, wherein the treatment of fibrosis comprises a decrease in the stage of fibrosis.
34) The method of any one of embodiments 25 to 33, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.
35) The method of any one of embodiments 1 to 34, wherein the ACC inhibitor is selected from the group consisting of:

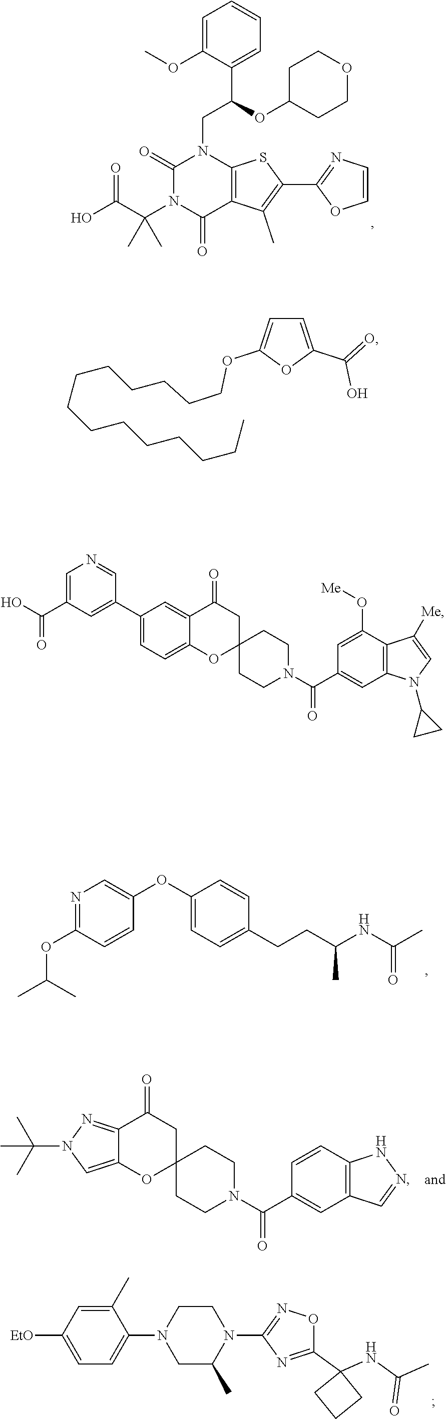

or a pharmaceutically acceptable salt of any of the foregoing.
36) The method of any one of embodiments 1 to 35, wherein the ACC inhibitor is

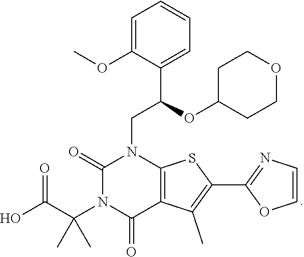 [SMILES: COc1ccccc1[C@H](Cn1c(=O)n(C(C)(C)C(=O)O)c(=O)c2c(C)c(-c3ncco3)sc21)OC1CCOCC1]

37) The method of any one of embodiments 1 to 36, wherein (a) and (b) are administered concurrently.
38) The method of any one of embodiments 1 to 36, wherein (a) and (b) are administered sequentially in either order.

39) The method of any one of embodiments 1 to 38, wherein the ACC inhibitor, or a pharmaceutically acceptable salt thereof, is administered at a dose from about 1 to about 400 mg.

40) The method of any one of embodiments 1 to 39, wherein the ACC inhibitor, or a pharmaceutically acceptable salt thereof, is administered at a dose from about 1 to about 150 mg.
41) The method of any one of embodiments 1 to 39, wherein the ACC inhibitor, or a pharmaceutically acceptable salt thereof, is administered at a dose from about 50 to about 250 mg.
42) The method of any one of embodiments 1 to 39, wherein the ACC inhibitor, or a pharmaceutically acceptable salt thereof, is administered at a dose of about 100 to about 300 mg.
43) The method of any one of embodiments 1 to 39, wherein the ACC inhibitor, or a pharmaceutically acceptable salt thereof, is administered at a dose of about 150 to about 400 mg.
44) The method of any one of embodiments 1 to 39, wherein the ACC inhibitor, or a pharmaceutically acceptable salt thereof, is administered at a dose of about 100 to about 200 mg.
45) The method of any one of embodiments 1 to 44, wherein the ACC inhibitor, or a pharmaceutically acceptable salt thereof, is administered to the subject twice a day, daily, every other day, three times a week, twice a week, weekly, every other week, twice a month, or monthly.
46) The method of any one of embodiments 1 to 45, wherein the ACC inhibitor, or a pharmaceutically acceptable salt thereof, is administered to the subject daily.
47) The method of any one of embodiments 1 to 46, wherein the adiponectin level in the subject is increased by at least about 30%, at least about 68%, at least about 175%, or at least about 200%.
48) The method of any one of embodiments 1 to 47, wherein the level of one or more biomarkers indicative of one or more of liver damage, inflammation, fibrosis, and/or cirrhosis is decreased.
49) The method of embodiment 48, wherein the increase is by at least about 175%.
50) The method of any one of embodiments 1 to 49, wherein the compound of Formula (I), a pharmaceutically acceptable salt thereof, is administered prophylactically.
51) The method of any one of embodiments 1 to 50, wherein the compound of Formula (I), or a pharmaceutically acceptable salt thereof, is administered at a dose from about 0.1 to about 15 mg.
52) The method of any one of embodiments 1 to 51, wherein the compound of Formula (I), or a pharmaceutically acceptable salt thereof, is administered at a dose from about 1 to about 10 mg.
53) The method of any one of embodiments 1 to 52, wherein the compound of Formula (I), or a pharmaceutically acceptable salt thereof, is administered at a dose from about 2 to about 6 mg.
54) The method of any one of embodiments 1 to 51, wherein the compound of Formula (I), or a pharmaceutically acceptable salt thereof, is administered at a dose from about 0.5 to about 3 mg.
55) The method of any one of embodiments 1 to 54, wherein the compound of Formula (I), or a pharmaceutically acceptable salt thereof, is administered at a dose of about 3 mg.
56) The method of any one of embodiments 1 to 54, wherein the compound of Formula (I), or a pharmaceutically acceptable salt thereof, is administered at a dose of about 2 mg.
57) The method of any one of embodiments 1 to 52 or 54, wherein the compound of Formula (I), or a pharmaceutically acceptable salt thereof, is administered at a dose of about 1 mg.
58) The method of any one of embodiments 1 to 57, wherein the compound of Formula (I), or a pharmaceutically acceptable salt thereof, is administered to the subject twice a day, daily, every other day, three times a week, twice a week, weekly, every other week, twice a month, or monthly.
59) The method of any one of embodiments 1 to 58, wherein the compound of Formula (I), or a pharmaceutically acceptable salt thereof, is administered to the subject daily.
60) The method of any one of embodiments 1 to 51, wherein the compound of Formula (I), or a pharmaceutically acceptable salt thereof, is administered to the subject daily and the dose of the compound of Formula (I) is about 3 mg.
61) The method of any one of embodiments 1 to 51, wherein the compound of Formula (I), or a pharmaceutically acceptable salt thereof, is administered at a dose from about 0.1 to about 10.0 mg per day.
62) The method of any one of embodiments 1 to 51 Or 61, wherein the compound of Formula (I), or a pharmaceutically acceptable salt thereof, is administered at a dose from about 0.1 to about 3 mg per day.
63) The method of any one of embodiments 1 to 51 or 61 to 62, wherein the compound of Formula (I), or a pharmaceutically acceptable salt thereof, is administered at a dose of about 0.5 mg per day.
64) The method of any one of embodiments 1 to 51 or 61 to 62, wherein the compound of Formula (I), or a pharmaceutically acceptable salt thereof, is administered at a dose of about 1 mg per day.
65) The method of any one of embodiments 1 to 51 or 61 to 62, wherein the compound of Formula (I), or a pharmaceutically acceptable salt thereof, is administered at a dose of about 2 mg per day.
66) The method of any one of embodiments 1 to 65, wherein the method further comprises performing a liver biopsy to determine the NAFLD activity score of the biopsy sample obtained from the subject.
67) A method of treating non-alcoholic fatty liver disease (NAFLD) in a subject in need thereof consisting essentially of administering to the subject

(a) the compound of Formula (I), or a pharmaceutically acceptable salt thereof, and

(b) an ACC inhibitor, or a pharmaceutically acceptable salt thereof,

wherein the amounts of (a) and (b) together are effective in treating NAFLD.

68) The method of any one of embodiments 1 to 67, wherein the compound of Formula (I) is in the form of a pharmaceutically acceptable salt.
69) The method of any one of embodiments 1 to 68, wherein the compound of Formula (I) is in the form of a besylate salt.
70) The method of any one of embodiments 1 to 67, wherein the compound of Formula (I) is in the form of a free base.
71) The method of any one of embodiments 1 to 70, wherein the ACC inhibitor is in the form of a pharmaceutically acceptable salt.
72) The method of any one of embodiments 1 to 70, wherein the ACC inhibitor is in the form of a free base.
73) A pharmaceutical composition comprising

(a) the compound of Formula (I), or a pharmaceutically acceptable salt thereof,

(b) an ACC inhibitor, or a pharmaceutically acceptable salt thereof, and
one or more pharmaceutical excipients.
   74) The pharmaceutical composition of embodiment 73, wherein the compound of Formula (I) is in the form of a pharmaceutically acceptable salt; and the ACC inhibitor is in the form of a pharmaceutically acceptable salt or a free base.
   75) A pharmaceutical combination comprising

(a) the compound of Formula (I), or a pharmaceutically acceptable salt thereof, and

(b) an ACC inhibitor, or a pharmaceutically acceptable salt thereof,

for concurrent or sequential administration for use in the treatment of non-alcoholic fatty liver disease (NAFLD).

76) The pharmaceutical combination of embodiment 75, wherein the compound of Formula (I) is in the form of a pharmaceutically acceptable salt; and the ACC inhibitor is in the form of a pharmaceutically acceptable salt or a free base.
77) A pharmaceutical combination of embodiment 75 or 76, further comprising at least one pharmaceutically acceptable carrier.
78) A method of treating non-alcoholic fatty liver disease (NAFLD) in a subject in need thereof comprising administering to the subject

(c) the compound of Formula (I), or a pharmaceutically acceptable salt thereof, and

(d) a caspase inhibitor, or a pharmaceutically acceptable salt thereof,
wherein the amounts of (a) and (b) together are effective in treating NAFLD.
   79) A method of treating a subject, the method comprising:

selecting a subject having non-alcoholic fatty liver disease (NAFLD); and

administering

(b) the compound of Formula (I), or a pharmaceutically acceptable salt thereof,

(b) a caspase inhibitor, or a pharmaceutically acceptable salt thereof, to the selected subject, wherein the amounts of (a) and (b) together are effective in treating NAFLD.

80) A method of treating a subject, the method comprising:

identifying a subject having non-alcoholic fatty liver disease (NAFLD); and

administering

(b) the compound of Formula (I), or a pharmaceutically acceptable salt thereof, and

(b) a caspase inhibitor, or a pharmaceutically acceptable salt thereof, to the selected subject, wherein the amounts of (a) and (b), together are effective in treating NAFLD.

81) The method of any one of embodiments 78 to 80, wherein the compound of Formula I is

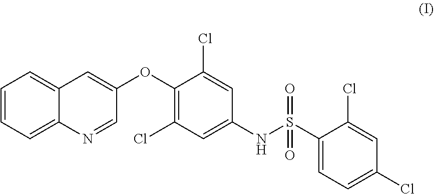 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
82) The method of any one of embodiments 78 to 81, wherein the treatment of NAFLD comprises a reduction in hepatic steatosis.
83) The method of any one of embodiments 78 to 82, wherein the treatment of NAFLD comprises a reduction in hepatic inflammation.
84) The method of any one of embodiments 78 to 83, wherein the NAFLD activity score (NAS) following administration is 7 or less.
85) The method of any one of embodiments 78 to 84, wherein the NAS is 5 or less.
86) The method of any one of embodiments 78 to 85, wherein the NAS is 3 or less.
87) The method of any one of embodiments 78 to 86, wherein the subject has hepatic cirrhosis associated with the NAFLD.
88) The method of any one of embodiments 78 to 87, wherein the subject has hepatic cirrhosis as a comorbidity.
89) The method of any one of embodiments 78 to 88, wherein the subject has hepatic cirrhosis caused by the NAFLD.
90) The method of any one of embodiments 78 to 89, wherein the NAFLD is NAFL with attendant liver cirrhosis.
91) The method of any one of embodiments 78 to 90, wherein the treatment of the NAFLD comprises treatment of liver cirrhosis.
92) The method of any one of embodiments 78 to 91, wherein the treatment of NAFLD decreases the level of serum bile acids in the subject.
93) The method of any one of embodiments 78 to 92, wherein the treatment of NAFLD comprises treatment of pruritus.
94) The method of any one of embodiments 78 to 93, wherein the NAFLD is simple nonalcoholic fatty liver (NAFL).
95) The method of embodiment 94, wherein the treatment of NAFL comprises treatment of pruritus.
96) The method of embodiment 94, wherein the treatment of NAFL decreases the level of serum bile acids in the subject.
97) The method of any one of embodiments 78 to 89 or 91 to 93, wherein the NAFLD is nonalcoholic steatohepatitis (NASH).
98) The method of any one of embodiments 78 to 89, 91 to 93, or 97, wherein the NAFLD is NASH with attendant liver cirrhosis.
99) The method of any one of embodiments 78 to 89, 91 to 93, or 97 to 98, wherein the treatment of NASH decreases the level of serum bile acids in the subject.
100) The method of any one of embodiments 78 to 89, 91 to 93, or 97 to 99, wherein the treatment of NASH comprises treatment of pruritus.
101) A method of treating fibrosis in a subject in need thereof comprising administering to the subject

(c) the compound of Formula (I), or a pharmaceutically acceptable salt thereof, and

(d) a caspase inhibitor, or a pharmaceutically acceptable salt thereof,
wherein the amounts of (a) and (b) together are effective in treating NAFLD.
   102) The method of embodiment 101, wherein the compound of Formula I is

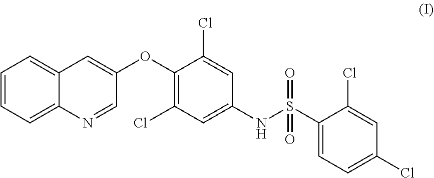 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
103) The method of embodiment 101 or 102, wherein the fibrosis is cirrhosis.
104) The method of any one of embodiments 101 to 103, wherein the fibrosis is associated with NAFLD.
105) The method of any one of embodiments 101 to 104, wherein the fibrosis is caused by NAFLD.
106) The method of any one of embodiments 101 to 105, wherein the NAFLD is NASH.
107) The method of any one of embodiments 101 to 106, wherein the treatment of fibrosis comprises a decrease in the stage of fibrosis, a lack of progression of the fibrosis, or a slowing in the progression of the fibrosis.
108) The method of any one of embodiments 101 to 107, wherein the treatment of fibrosis comprises a decrease in the stage of fibrosis.
109) The method of any one of embodiments 101 to 108, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0. 110) The method of any one of embodiments 78 to 109, wherein the caspase inhibitor is selected from the group consisting of:

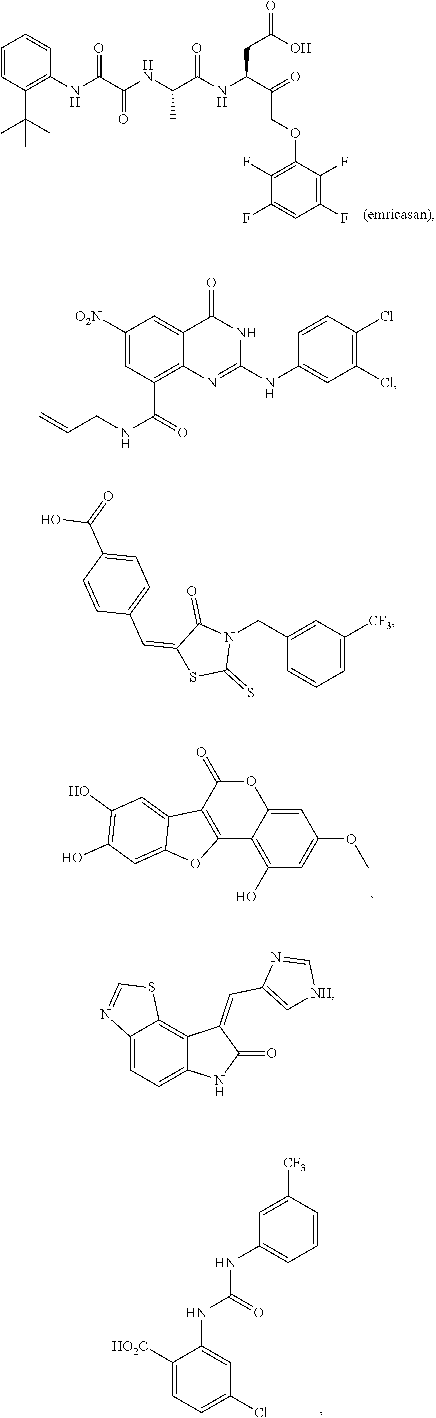 [SMILES: C[C@H](NC(=O)C(=O)Nc1ccccc1C(C)(C)C)C(=O)N[C@@H](CC(=O)O)C(=O)COc1c(F)c(F)cc(F)c1F ; C=CCNC(=O)c1cc([N+](=O)[O-])cc2c(=O)[nH]c(Nc3ccc(Cl)c(Cl)c3)nc12 ; O=C(O)c1ccc(/C=C2/SC(=S)N(Cc3cccc(C(F)(F)F)c3)C2=O)cc1 ; COc1cc(O)c2c(c1)oc(=O)c1c3cc(O)c(O)cc3oc21 ; O=C1Nc2ccc3ncsc3c2/C1=C/c1c[nH]cn1 ; O=C(Nc1cccc(C(F)(F)F)c1)Nc1cc(Cl)ccc1C(=O)O]

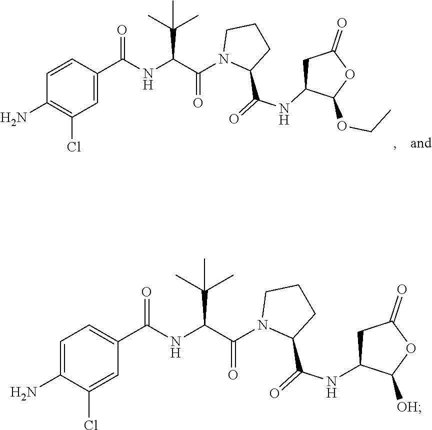 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C ; CC(C)(C)[C@H](NC(=O)c1ccc(N)c(Cl)c1)C(=O)N1CCC[C@H]1C(=O)N[C@H]1CC(=O)O[C@H]1O]

or a pharmaceutically acceptable salt of any of the foregoing.
111) The method of any one of embodiments 78 to 110, wherein the caspase inhibitor is emricasan.
112) The method of any one of embodiments 78 to 111, wherein (a) and (b) are administered concurrently.
113) The method of any one of embodiments 78 to 111, wherein (a) and (b) are administered sequentially in either order.
114) The method of any one of embodiments 78 to 113, wherein the caspase inhibitor, or a pharmaceutically acceptable salt thereof, is administered at a dose from about 1 to about 500 mg.
115) The method of any one of embodiments 78 to 114, wherein the caspase inhibitor, or a pharmaceutically acceptable salt thereof, is administered at a dose from about 1 to about 10 mg.
116) The method of any one of embodiments 78 to 114, wherein the caspase inhibitor, or a pharmaceutically acceptable salt thereof, is administered at a dose from about 5 to about 25 mg.
117) The method of any one of embodiments 78 to 114, wherein the caspase inhibitor, or a pharmaceutically acceptable salt thereof, is administered at a dose of about 20 to about 100 mg.
118) The method of any one of embodiments 78 to 114, wherein the caspase inhibitor, or a pharmaceutically acceptable salt thereof, is administered at a dose of about 25 to about 100 mg.
119) The method of any one of embodiments 78 to 114, wherein the caspase inhibitor, or a pharmaceutically acceptable salt thereof, is administered at a dose of about 50 to about 200 mg.
120) The method of any one of embodiments 78 to 114, wherein the caspase inhibitor, or a pharmaceutically acceptable salt thereof, is administered at a dose of about 100 to about 300 mg.
121) The method of any one of embodiments 78 to 114, wherein the caspase inhibitor, or a pharmaceutically acceptable salt thereof, is administered at a dose of about 200 to about 350 mg.
122) The method of any one of embodiments 78 to 121, wherein the caspase inhibitor, or a pharmaceutically acceptable salt thereof, is administered to the subject twice a day, daily, every other day, three times a week, twice a week, weekly, every other week, twice a month, or monthly.
123) The method of any one of embodiments 78 to 122, wherein the caspase inhibitor, or a pharmaceutically acceptable salt thereof, is administered to the subject daily.
124) The method of any one of embodiments 78 to 123, wherein the adiponectin level in the subject is increased by at least about 30%, at least about 68%, at least about 175%, or at least about 200%.
125) The method of any one of embodiments 78 to 124, wherein the level of one or more biomarkers indicative of one or more of liver damage, inflammation, fibrosis, and/or cirrhosis is decreased.
126) The method of embodiment 125, wherein the increase is by at least about 175%.
127) The method of any one of embodiments 78 to 126, wherein the compound of Formula (I), a pharmaceutically acceptable salt thereof, is administered prophylactically.
128) The method of any one of embodiments 78 to 127, wherein the compound of Formula (I), or a pharmaceutically acceptable salt thereof, is administered at a dose from about 0.1 to about 15 mg.
129) The method of any one of embodiments 78 to 128, wherein the compound of Formula (I), or a pharmaceutically acceptable salt thereof, is administered at a dose from about 1 to about 10 mg.
130) The method of any one of embodiments 78 to 129, wherein the compound of Formula (I), or a pharmaceutically acceptable salt thereof, is administered at a dose from about 2 to about 6 mg.
131) The method of any one of embodiments 78 to 128, wherein the compound of Formula (I), or a pharmaceutically acceptable salt thereof, is administered at a dose from about 0.5 to about 3 mg.
132) The method of any one of embodiments 78 to 131, wherein the compound of Formula (I), or a pharmaceutically acceptable salt thereof, is administered at a dose of about 3 mg.
133) The method of any one of embodiments 78 to 131, wherein the compound of Formula (I), or a pharmaceutically acceptable salt thereof, is administered at a dose of about 2 mg.
134) The method of any one of embodiments 78 to 129 or 131, wherein the compound of Formula (I), or a pharmaceutically acceptable salt thereof, is administered at a dose of about 1 mg.
135) The method of any one of embodiments 78 to 134, wherein the compound of Formula (I), or a pharmaceutically acceptable salt thereof, is administered to the subject twice a day, daily, every other day, three times a week, twice a week, weekly, every other week, twice a month, or monthly.
136) The method of any one of embodiments 78 to 135, wherein the compound of Formula (I), or a pharmaceutically acceptable salt thereof, is administered to the subject daily.
137) The method of any one of embodiments 78 to 128, wherein the compound of Formula (I), or a pharmaceutically acceptable salt thereof, is administered to the subject daily and the dose of the compound of Formula (I) is about 3 mg.
138) The method of any one of embodiments 78 to 128, wherein the compound of Formula (I), or a pharmaceutically acceptable salt thereof, is administered at a dose from about 0.1 to about 10.0 mg per day.
139) The method of any one of embodiments 78 to 128 or 138, wherein the compound of Formula (I), or a pharmaceutically acceptable salt thereof, is administered at a dose from about 0.1 to about 3 mg per day.
140) The method of any one of embodiments 78 to 128 or 138 to 139, wherein the compound of Formula (I), or a pharmaceutically acceptable salt thereof, is administered at a dose of about 0.5 mg per day.
141) The method of any one of embodiments 78 to 128 or 138 to 139, wherein the compound of Formula (I), or a pharmaceutically acceptable salt thereof, is administered at a dose of about 1 mg per day.
142) The method of any one of embodiments 78 to 128 or 138 to 139, wherein the compound of Formula (I), or a pharmaceutically acceptable salt thereof, is administered at a dose of about 2 mg per day.
143) The method of any one of embodiments 78 to 142, wherein the method further comprises performing a liver biopsy to determine the NAFLD activity score of the biopsy sample obtained from the subject.
144) A method of treating non-alcoholic fatty liver disease (NAFLD) in a subject in need thereof consisting essentially of administering to the subject

(c) the compound of Formula (I), or a pharmaceutically acceptable salt thereof, and

(d) a caspase inhibitor, or a pharmaceutically acceptable salt thereof,
wherein the amounts of (a) and (b) together are effective in treating NAFLD.
   145) The method of any one of embodiments 78 to 144, wherein the compound of Formula (I) is in the form of a pharmaceutically acceptable salt.
   146) The method of any one of embodiments 78 to 145, wherein the compound of Formula (I) is in the form of a besylate salt.
   147) The method of any one of embodiments 78 to 144, wherein the compound of Formula (I) is in the form of a free base.
   148) The method of any one of embodiments 78 to 147, wherein the caspase inhibitor is in the form of a pharmaceutically acceptable salt.
   149) The method of any one of embodiments 78 to 147, wherein the caspase inhibitor is in the form of a free base.
   150) A pharmaceutical composition comprising

(c) the compound of Formula (I), or a pharmaceutically acceptable salt thereof,

(d) a caspase inhibitor, or a pharmaceutically acceptable salt thereof, and
one or more pharmaceutical excipients.
   151) The pharmaceutical composition of embodiment 150, wherein the compound of Formula (I) is in the form of a pharmaceutically acceptable salt; and the caspase inhibitor is in the form of a pharmaceutically acceptable salt or a free base.
   152) A pharmaceutical combination comprising

(c) the compound of Formula (I), or a pharmaceutically acceptable salt thereof, and

(d) a caspase inhibitor, or a pharmaceutically acceptable salt thereof,

for concurrent or sequential administration for use in the treatment of non-alcoholic fatty liver disease (NAFLD).

153) The pharmaceutical combination of embodiment 152, wherein the compound of Formula (I) is in the form of a pharmaceutically acceptable salt; and the caspase inhibitor is in the form of a pharmaceutically acceptable salt or a free base.
154) A pharmaceutical combination of embodiment 152 or 153, further comprising at least one pharmaceutically acceptable carrier.
155) A method of treating non-alcoholic fatty liver disease (NAFLD) in a subject in need thereof comprising administering to the subject

(a) the compound of Formula (I), or a pharmaceutically acceptable salt thereof, and

(b) an MCR antagonist, or a pharmaceutically acceptable salt thereof,
wherein the amounts of (a) and (b) together are effective in treating NAFLD.
   156) A method of treating a subject, the method comprising:

selecting a subject having non-alcoholic fatty liver disease (NAFLD); and

administering

(a) the compound of Formula (I), or a pharmaceutically acceptable salt thereof,

(b) an MCR antagonist, or a pharmaceutically acceptable salt thereof, to the selected subject, wherein the amounts of (a) and (b) together are effective in treating NAFLD.

157) A method of treating a subject, the method comprising:

identifying a subject having non-alcoholic fatty liver disease (NAFLD); and

administering

(a) the compound of Formula (I), or a pharmaceutically acceptable salt thereof, and

(b) an MCR antagonist, or a pharmaceutically acceptable salt thereof, to the selected subject, wherein the amounts of (a) and (b), together are effective in treating NAFLD.

158) The method of any one of embodiments 155 to 157, wherein the compound of Formula I is

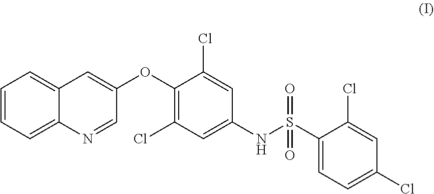 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
159) The method of any one of embodiments 155 to 158, wherein the treatment of NAFLD comprises a reduction in hepatic steatosis.
160) The method of any one of embodiments 155 to 159, wherein the treatment of NAFLD comprises a reduction in hepatic inflammation.
161) The method of any one of embodiments 155 to 160, wherein the NAFLD activity score (NAS) following administration is 7 or less.
162) The method of any one of embodiments 155 to 161, wherein the NAS is 5 or less.
163) The method of any one of embodiments 155 to 162, wherein the NAS is 3 or less.
164) The method of any one of embodiments 155 to 163, wherein the subject has hepatic cirrhosis associated with the NAFLD.
165) The method of any one of embodiments 155 to 164, wherein the subject has hepatic cirrhosis as a comorbidity.
166) The method of any one of embodiments 155 to 165, wherein the subject has hepatic cirrhosis caused by the NAFLD.
167) The method of any one of embodiments 155 to 166, wherein the NAFLD is NAFL with attendant liver cirrhosis.
168) The method of any one of embodiments 155 to 167, wherein the treatment of the NAFLD comprises treatment of liver cirrhosis.
169) The method of any one of embodiments 155 to 168, wherein the treatment of NAFLD decreases the level of serum bile acids in the subject.
170) The method of any one of embodiments 155 to 169, wherein the treatment of NAFLD comprises treatment of pruritus.
171) The method of any one of embodiments 155 to 163, wherein the NAFLD is simple nonalcoholic fatty liver (NAFL).
172) The method of embodiment 171, wherein the treatment of NAFL comprises treatment of pruritus.
173) The method of embodiment 171, wherein the treatment of NAFL decreases the level of serum bile acids in the subject.
174) The method of any one of embodiments 155 to 166 or 168 to 170, wherein the NAFLD is nonalcoholic steatohepatitis (NASH).
175) The method of any one of embodiments 155 to 166, 168 to 170, or 174, wherein the NAFLD is NASH with attendant liver cirrhosis.
176) The method of any one of embodiments 155 to 166, 168 to 170, or 174 to 175, wherein the treatment of NASH decreases the level of serum bile acids in the subject.
177) The method of any one of embodiments 155 to 166, 168 to 170, or 174 to 176, wherein the treatment of NASH comprises treatment of pruritus.
178) A method of treating fibrosis in a subject in need thereof comprising administering to the subject (a) the compound of Formula (I), or a pharmaceutically acceptable salt thereof, and (b) an MCR antagonist, or a pharmaceutically acceptable salt thereof, wherein the amounts of (a) and (b) together are effective in treating NAFLD.
179) The method of any one of embodiment 178, wherein the compound of Formula I is

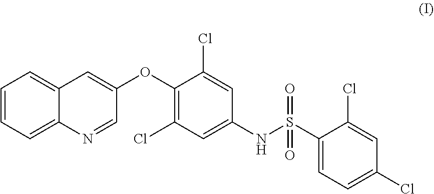 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
180) The method of embodiment 178 or 179, wherein the fibrosis is cirrhosis.
181) The method of any one of embodiments 178 to 180, wherein the fibrosis is associated with NAFLD.
182) The method of any one of embodiments 178 to 181, wherein the fibrosis is caused by NAFLD.
183) The method of any one of embodiments 178 to 182, wherein the NAFLD is NASH.
184) The method of any one of embodiments 178 to 183, wherein the treatment of fibrosis comprises a decrease in the stage of fibrosis, a lack of progression of the fibrosis, or a slowing in the progression of the fibrosis.
185) The method of any one of embodiments 178 to 184, wherein the treatment of fibrosis comprises a decrease in the stage of fibrosis.
186) The method of any one of embodiments 178 to 185, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.
187) The method of any one of embodiments 155 to 186, wherein the MCR antagonist is selected from the group consisting of: spironolactone, finerenone, eplerenone, canrenone, mexrenone, amlodipine, apararenone, benidipine, esaxerenone, felodipine, nefedipine, nimodipine, and nitrendipine; or a pharmaceutically acceptable salt of any of the foregoing.
188) The method of any one of embodiments 155 to 187, wherein the MCR antagonist is selected from the group consisting of: spironolactone, finerenone, eplerenone, canrenone, and mexrenone; or a pharmaceutically acceptable salt, of any of the foregoing.
189) The method of any one of embodiments 155 to 187, wherein the MCR antagonist is selected from the group consisting of: amlodipine, apararenone, benidipine, esaxerenone, felodipine, nefedipine, nimodipine, and nitrendipine; or a pharmaceutically acceptable salt, of any of the foregoing.
190) The method of any one of embodiments 155 to 189, wherein the MCR antagonist is spironolactone.
191) The method of any one of embodiments 155 to 190, wherein (a) and (b) are administered concurrently.
192) The method of any one of embodiments 155 to 190, wherein (a) and (b) are administered sequentially in either order.
193) The method of any one of embodiments 155 to 192, wherein the MCR antagonist, or a pharmaceutically acceptable salt thereof, is administered at a dose from about 0.5 to about 150 mg.
194) The method of any one of embodiments 155 to 193, wherein the MCR antagonist, or a pharmaceutically acceptable salt thereof, is administered at a dose from about 8 to about 20 mg.
195) The method of any one of embodiments 155 to 193, wherein the MCR antagonist, or a pharmaceutically acceptable salt thereof, is administered at a dose from about 15 to about 30 mg.
196) The method of any one of embodiments 155 to 193, wherein the MCR antagonist, or a pharmaceutically acceptable salt thereof, is administered at a dose of about 20 to about 40 mg.
197) The method of any one of embodiments 155 to 193, wherein the MCR antagonist, or a pharmaceutically acceptable salt thereof, is administered at a dose of about 25 to about 50 mg.
198) The method of any one of embodiments 155 to 193, wherein the MCR antagonist, or a pharmaceutically acceptable salt thereof, is administered at a dose of about 40 to about 75 mg.
199) The method of any one of embodiments 155 to 198, wherein the MCR antagonist, or a pharmaceutically acceptable salt thereof, is administered to the subject twice a day, daily, every other day, three times a week, twice a week, weekly, every other week, twice a month, or monthly.
200) The method of any one of embodiments 155 to 199, wherein the MCR antagonist, or a pharmaceutically acceptable salt thereof, is administered to the subject daily.
201) The method of any one of embodiments 155 to 200, wherein the adiponectin level in the subject is increased by at least about 30%, at least about 68%, at least about 175%, or at least about 200%.
202) The method of any one of embodiments 155 to 201, wherein the level of one or more biomarkers indicative of one or more of liver damage, inflammation, fibrosis, and/or cirrhosis is decreased.
203) The method of embodiment 202, wherein the increase is by at least about 175%.
204) The method of any one of embodiments 155 to 203, wherein the compound of Formula (I), a pharmaceutically acceptable salt thereof, is administered prophylactically.
205) The method of any one of embodiments 155 to 204, wherein the compound of Formula (I), or a pharmaceutically acceptable salt thereof, is administered at a dose from about 0.1 to about 15 mg.
206) The method of any one of embodiments 155 to 205, wherein the compound of Formula (I), or a pharmaceutically acceptable salt thereof, is administered at a dose from about 1 to about 10 mg.
207) The method of any one of embodiments 155 to 206, wherein the compound of Formula (I), or a pharmaceutically acceptable salt thereof, is administered at a dose from about 2 to about 6 mg.
208) The method of any one of embodiments 155 to 205, wherein the compound of Formula (I), or a pharmaceutically acceptable salt thereof, is administered at a dose from about 0.5 to about 3 mg.
209) The method of any one of embodiments 155 to 208, wherein the compound of Formula (I), or a pharmaceutically acceptable salt thereof, is administered at a dose of about 3 mg.
210) The method of any one of embodiments 155 to 208, wherein the compound of Formula (I), or a pharmaceutically acceptable salt thereof, is administered at a dose of about 2 mg.
211) The method of any one of embodiments 155 to 206 or 208, wherein the compound of Formula (I), or a pharmaceutically acceptable salt thereof, is administered at a dose of about 1 mg.
212) The method of any one of embodiments 155 to 211, wherein the compound of Formula (I), or a pharmaceutically acceptable salt thereof, is administered to the subject twice a day, daily, every other day, three times a week, twice a week, weekly, every other week, twice a month, or monthly.
213) The method of any one of embodiments 155 to 212, wherein the compound of Formula (I), or a pharmaceutically acceptable salt thereof, is administered to the subject daily.
214) The method of any one of embodiments 155 to 205, wherein the compound of Formula (I), or a pharmaceutically acceptable salt thereof, is administered to the subject daily and the dose of the compound of Formula (I) is about 3 mg.
215) The method of any one of embodiments 155 to 205, wherein the compound of Formula (I), or a pharmaceutically acceptable salt thereof, is administered at a dose from about 0.1 to about 10.0 mg per day.
216) The method of any one of embodiments 155 to 205 or 215, wherein the compound of Formula (I), or a pharmaceutically acceptable salt thereof, is administered at a dose from about 0.1 to about 3 mg per day.
217) The method of any one of embodiments 155 to 205 or 215 to 216, wherein the compound of Formula (I), or a pharmaceutically acceptable salt thereof, is administered at a dose of about 0.5 mg per day.
218) The method of any one of embodiments 155 to 205 or 215 to 216, wherein the compound of Formula (I), or a pharmaceutically acceptable salt thereof, is administered at a dose of about 1 mg per day.
219) The method of any one of embodiments 155 to 205 or 215 to 216, wherein the compound of Formula (I), or a pharmaceutically acceptable salt thereof, is administered at a dose of about 2 mg per day.
220) The method of any one of embodiments 155 to 219, wherein the method further comprises performing a liver biopsy to determine the NAFLD activity score of the biopsy sample obtained from the subject.
221) A method of treating non-alcoholic fatty liver disease (NAFLD) in a subject in need thereof consisting essentially of administering to the subject

(a) the compound of Formula (I), or a pharmaceutically acceptable salt thereof, and

(b) an MCR antagonist, or a pharmaceutically acceptable salt thereof,
wherein the amounts of (a) and (b) together are effective in treating NAFLD.
   222) The method of any one of embodiments 155 to 221, wherein the compound of Formula (I) is in the form of a pharmaceutically acceptable salt.
   223) The method of any one of embodiments 155 to 222, wherein the compound of Formula (I) is in the form of a besylate salt.
   224) The method of any one of embodiments 155 to 221, wherein the compound of Formula (I) is in the form of a free base.
   225) The method of any one of embodiments 155 to 224, wherein the MCR antagonist is in the form of a pharmaceutically acceptable salt.
   226) The method of any one of embodiments 155 to 224, wherein the MCR antagonist is in the form of a free base.
   227) A pharmaceutical composition comprising

(a) the compound of Formula (I), or a pharmaceutically acceptable salt thereof,

(b) an MCR antagonist, or a pharmaceutically acceptable salt thereof, and
one or more pharmaceutical excipients.
   228) The pharmaceutical composition of embodiment 227, wherein the compound of Formula (I) is in the form of a pharmaceutically acceptable salt; and the MCR antagonist is in the form of a pharmaceutically acceptable salt or a free base.
   229) A pharmaceutical combination comprising

(a) the compound of Formula (I), or a pharmaceutically acceptable salt thereof, and

(b) an MCR antagonist, or a pharmaceutically acceptable salt thereof,

for concurrent or sequential administration for use in the treatment of non-alcoholic fatty liver disease (NAFLD).

230) The pharmaceutical combination of embodiment 229, wherein the compound of Formula (I) is in the form of a pharmaceutically acceptable salt; and the MCR antagonist is in the form of a pharmaceutically acceptable salt or a free base.
231) A pharmaceutical combination of embodiment 229 or 230, further comprising at least one pharmaceutically acceptable carrier.
232) A method of treating non-alcoholic fatty liver disease (NAFLD) in a subject in need thereof comprising administering to the subject

(c) the compound of Formula (I), or a pharmaceutically acceptable salt thereof, and

(d) a statin, or a pharmaceutically acceptable salt thereof,
wherein the amounts of (a) and (b) together are effective in treating NAFLD.
   233) A method of treating a subject, the method comprising:

selecting a subject having non-alcoholic fatty liver disease (NAFLD); and

administering

(b) the compound of Formula (I), or a pharmaceutically acceptable salt thereof,

(b) a statin, or a pharmaceutically acceptable salt thereof, to the selected subject, wherein the amounts of (a) and (b) together are effective in treating NAFLD.

234) A method of treating a subject, the method comprising:

identifying a subject having non-alcoholic fatty liver disease (NAFLD); and

administering

(b) the compound of Formula (I), or a pharmaceutically acceptable salt thereof, and

(b) a statin, or a pharmaceutically acceptable salt thereof, to the selected subject, wherein the amounts of (a) and (b), together are effective in treating NAFLD.

235) The method of any one of embodiments 231 to 233, wherein the compound of Formula I

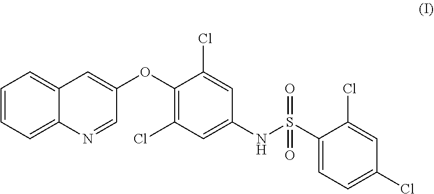 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
236) The method of any one of embodiments 231 to 234 wherein the statin, or a pharmaceutically acceptable salt thereof, is atorvastatin, cerivastatin, fluvastatin, lovastatin, mevastatin, pitavastatin, pravastatin, rosuvastatin, or simvastatin, or a pharmaceutically acceptable salt thereof.
237) The method of any one of embodiments 231 to 235, wherein the treatment of NAFLD comprises a reduction in hepatic steatosis.
238) The method of any one of embodiments 231 to 236, wherein the treatment of NAFLD comprises a reduction in hepatic inflammation.
239) The method of any one of embodiments 231 to 237, wherein the NAFLD activity score (NAS) following administration is 7 or less.
240) The method of any one of embodiments 231 to 238, wherein the NAS is 5 or less.
241) The method of any one of embodiments 231 to 239, wherein the NAS is 3 or less.
242) The method of any one of embodiments 231 to 240, wherein the subject has hepatic cirrhosis associated with the NAFLD.
243) The method of any one of embodiments 231 to 241, wherein the subject has hepatic cirrhosis as a comorbidity.
244) The method of any one of embodiments 231 to 242, wherein the subject has hepatic cirrhosis caused by the NAFLD.
245) The method of any one of embodiments 231 to 243, wherein the NAFLD is NAFL with attendant liver cirrhosis.
246) The method of any one of embodiments 231 to 244, wherein the treatment of the NAFLD comprises treatment of liver cirrhosis.
247) The method of any one of embodiments 231 to 245, wherein the treatment of NAFLD decreases the level of serum bile acids in the subject.
248) The method of any one of embodiments 231 to 246, wherein the treatment of NAFLD comprises treatment of pruritus.
249) The method of any one of embodiments 231 to 240, wherein the NAFLD is simple nonalcoholic fatty liver (NAFL).
250) The method of embodiment 248, wherein the treatment of NAFL comprises treatment of pruritus.
251) The method of embodiment 248, wherein the treatment of NAFL decreases the level of serum bile acids in the subject.
252) The method of any one of embodiments 231 to 243 or 245 to 247, wherein the NAFLD is nonalcoholic steatohepatitis (NASH).
253) The method of any one of embodiments 231 to 243, 245 to 247, or 251, wherein the NAFLD is NASH with attendant liver cirrhosis.
254) The method of any one of embodiments 231 to 243, 245 to 247, or 251 to 252, wherein the treatment of NASH decreases the level of serum bile acids in the subject.
255) The method of any one of embodiments 231 to 243, 245 to 247, or 251 to 253, wherein the treatment of NASH comprises treatment of pruritus.
256) A method of treating fibrosis in a subject in need thereof comprising administering to the subject

(a) the compound of Formula (I), or a pharmaceutically acceptable salt thereof, and

(b) a statin, or a pharmaceutically acceptable salt thereof,
wherein the amounts of (a) and (b) together are effective in treating NAFLD.
   257) The method of embodiment 255, wherein the compound of Formula I is

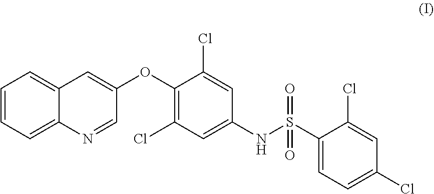 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
258) The method of embodiment 255 or 256, wherein the statin, or a pharmaceutically acceptable salt thereof, is atorvastatin, cerivastatin, fluvastatin, lovastatin, mevastatin, pitavastatin, pravastatin, rosuvastatin, or simvastatin, or a pharmaceutically acceptable salt thereof.
259) The method of any one of embodiments 255 to 257, wherein the fibrosis is cirrhosis.
260) The method of any one of embodiments 255 to 258, wherein the fibrosis is associated with NAFLD.
261) The method of any one of embodiments 255 to 259, wherein the fibrosis is caused by NAFLD.
262) The method of any one of embodiments 255 to 260, wherein the NAFLD is NASH.
263) The method of any one of embodiments 255 to 261, wherein the treatment of fibrosis comprises a decrease in the stage of fibrosis, a lack of progression of the fibrosis, or a slowing in the progression of the fibrosis.
264) The method of any one of embodiments 255 to 262, wherein the treatment of fibrosis comprises a decrease in the stage of fibrosis.
265) The method of any one of embodiments 255 to 263, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.
266) The method of any one of embodiments 231 to 264, wherein the statin is selected from the group consisting of: atorvastatin, rosuvastatin, and simvastatin, or a pharmaceutically acceptable salt thereof.
267) The method of any one of embodiments 231 to 265, wherein the statin is atorvastatin.
268) The method of any one of embodiments 231 to 266, wherein (a) and (b) are administered concurrently.
269) The method of any one of embodiments 231 to 266, wherein (a) and (b) are administered sequentially in either order.
270) The method of any one of embodiments 231 to 268, wherein the statin, or a pharmaceutically acceptable salt thereof, is administered at a dose from about 1 to about 80 mg.
271) The method of any one of embodiments 231 to 269, wherein the statin, or a pharmaceutically acceptable salt thereof, is administered at a dose from about 1 to about 20 mg.
272) The method of any one of embodiments 231 to 269, wherein the statin, or a pharmaceutically acceptable salt thereof, is administered at a dose from about 15 to about 50 mg.
273) The method of any one of embodiments 231 to 269, wherein the statin, or a pharmaceutically acceptable salt thereof, is administered at a dose of about 5 to about 30 mg.
274) The method of any one of embodiments 231 to 269, wherein the statin, or a pharmaceutically acceptable salt thereof, is administered at a dose of 10 to about 40 mg.
275) The method of any one of embodiments 231 to 269, wherein the statin, or a pharmaceutically acceptable salt thereof, is administered at a dose of about 20 to about 60 mg.
276) The method of any one of embodiments 231 to 274, wherein the statin, or a pharmaceutically acceptable salt thereof, is administered to the subject twice a day, daily, every other day, three times a week, twice a week, weekly, every other week, twice a month, or monthly.
277) The method of any one of embodiments 231 to 275, wherein the statin, or a pharmaceutically acceptable salt thereof, is administered to the subject daily.
278) The method of any one of embodiments 231 to 276, wherein the adiponectin level in the subject is increased by at least about 30%, at least about 68%, at least about 175%, or at least about 200%.
279) The method of any one of embodiments 231 to 277, wherein the level of one or more biomarkers indicative of one or more of liver damage, inflammation, fibrosis, and/or cirrhosis is decreased.
280) The method of embodiment 278, wherein the increase is by at least about 175%.
281) The method of any one of embodiments 231 to 279, wherein the compound of Formula (I), a pharmaceutically acceptable salt thereof, is administered prophylactically.
282) The method of any one of embodiments 231 to 280, wherein the compound of Formula (I), or a pharmaceutically acceptable salt thereof, is administered at a dose from about 0.1 to about 15 mg.
283) The method of any one of embodiments 231 to 281, wherein the compound of Formula (I), or a pharmaceutically acceptable salt thereof, is administered at a dose from about 1 to about 10 mg.
284) The method of any one of embodiments 231 to 282, wherein the compound of Formula (I), or a pharmaceutically acceptable salt thereof, is administered at a dose from about 2 to about 6 mg.
285) The method of any one of embodiments 231 to 281, wherein the compound of Formula (I), or a pharmaceutically acceptable salt thereof, is administered at a dose from about 0.5 to about 3 mg.
286) The method of any one of embodiments 231 to 284, wherein the compound of Formula (I), or a pharmaceutically acceptable salt thereof, is administered at a dose of about 3 mg.
287) The method of any one of embodiments 231 to 284, wherein the compound of Formula (I), or a pharmaceutically acceptable salt thereof, is administered at a dose of about 2 mg.
288) The method of any one of embodiments 231 to 282 or 284, wherein the compound of Formula (I), or a pharmaceutically acceptable salt thereof, is administered at a dose of about 1 mg.
289) The method of any one of embodiments 231 to 287, wherein the compound of Formula (I), or a pharmaceutically acceptable salt thereof, is administered to the subject twice a day, daily, every other day, three times a week, twice a week, weekly, every other week, twice a month, or monthly.
290) The method of any one of embodiments 231 to 288, wherein the compound of Formula (I), or a pharmaceutically acceptable salt thereof, is administered to the subject daily.
291) The method of any one of embodiments 231 to 281, wherein the compound of Formula (I), or a pharmaceutically acceptable salt thereof, is administered to the subject daily and the dose of the compound of Formula (I) is about 3 mg.
292) The method of any one of embodiments 231 to 281, wherein the compound of Formula (I), or a pharmaceutically acceptable salt thereof, is administered at a dose from about 0.1 to about 10.0 mg per day.
293) The method of any one of embodiments 231 to 281 or 291, wherein the compound of Formula (I), or a pharmaceutically acceptable salt thereof, is administered at a dose from about 0.1 to about 3 mg per day.
294) The method of any one of embodiments 231 to 281 or 291 to 292, wherein the compound of Formula (I), or a pharmaceutically acceptable salt thereof, is administered at a dose of about 0.5 mg per day.
295) The method of any one of embodiments 231 to 281 or 291 to 292, wherein the compound of Formula (I), or a pharmaceutically acceptable salt thereof, is administered at a dose of about 1 mg per day.
296) The method of any one of embodiments 231 to 281 or 291 to 292, wherein the compound of Formula (I), or a pharmaceutically acceptable salt thereof, is administered at a dose of about 2 mg per day.
297) The method of any one of embodiments 231 to 295, wherein the method further comprises performing a liver biopsy to determine the NAFLD activity score of the biopsy sample obtained from the subject.
298) A method of treating non-alcoholic fatty liver disease (NAFLD) in a subject in need thereof consisting essentially of administering to the subject

(c) the compound of Formula (I), or a pharmaceutically acceptable salt thereof, and

(d) a statin, or a pharmaceutically acceptable salt thereof,
wherein the amounts of (a) and (b) together are effective in treating NAFLD.
   299) The method of any one of embodiments 231 to 297, wherein the compound of Formula (I) is in the form of a pharmaceutically acceptable salt.
   300) The method of any one of embodiments 231 to 298, wherein the compound of Formula (I) is in the form of a besylate salt.
   301) The method of any one of embodiments 231 to 297, wherein the compound of Formula (I) is in the form of a free base.
   302) The method of any one of embodiments 231 to 300, wherein the statin is in the form of a pharmaceutically acceptable salt.
   303) The method of any one of embodiments 231 to 300, wherein the statin is in the form of a free base.
   304) A pharmaceutical composition comprising

(c) the compound of Formula (I), or a pharmaceutically acceptable salt thereof,

(d) a statin, or a pharmaceutically acceptable salt thereof, and
one or more pharmaceutical excipients.
   305) The pharmaceutical composition of embodiment 303, wherein the compound of Formula (I) is in the form of a pharmaceutically acceptable salt; and the statin is in the form of a pharmaceutically acceptable salt or a free base.
   306) A pharmaceutical combination comprising

(c) the compound of Formula (I), or a pharmaceutically acceptable salt thereof, and

(d) a statin, or a pharmaceutically acceptable salt thereof,

for concurrent or sequential administration for use in the treatment of non-alcoholic fatty liver disease (NAFLD).

307) The pharmaceutical combination of embodiment 305, wherein the compound of Formula (I) is in the form of a pharmaceutically acceptable salt; and the statin is in the form of a pharmaceutically acceptable salt or a free base.
308) A pharmaceutical combination of embodiment 305 or 306, further comprising at least one pharmaceutically acceptable carrier.
309) A method of treating non-alcoholic fatty liver disease (NAFLD) in a subject in need thereof comprising administering to the subject

(e) the compound of Formula (I), or a pharmaceutically acceptable salt thereof, and

(f) a PCSK9 inhibitor,
wherein the amounts of (a) and (b) together are effective in treating NAFLD.
   310) A method of treating a subject, the method comprising:

selecting a subject having non-alcoholic fatty liver disease (NAFLD); and

administering

(c) the compound of Formula (I), or a pharmaceutically acceptable salt thereof,

(b) a PCSK9 inhibitor, to the selected subject, wherein the amounts of (a) and (b) together are effective in treating NAFLD.

311) A method of treating a subject, the method comprising:

identifying a subject having non-alcoholic fatty liver disease (NAFLD); and

administering

(c) the compound of Formula (I), or a pharmaceutically acceptable salt thereof, and

(b) a PCSK9 inhibitor, to the selected subject, wherein the amounts of (a) and (b), together are effective in treating NAFLD.

312) The method of any one of embodiments 308 to 310, wherein the compound of Formula I is

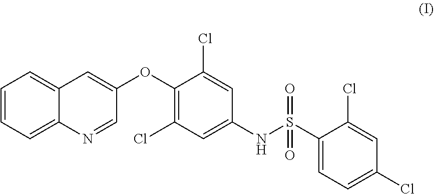 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
313) The method of any one of embodiments 308 to 311, wherein the treatment of NAFLD comprises a reduction in hepatic steatosis.
314) The method of any one of embodiments 308 to 312, wherein the treatment of NAFLD comprises a reduction in hepatic inflammation.
315) The method of any one of embodiments 308 to 313, wherein the NAFLD activity score (NAS) following administration is 7 or less.
316) The method of any one of embodiments 308 to 314, wherein the NAS is 5 or less.
317) The method of any one of embodiments 308 to 315, wherein the NAS is 3 or less.
318) The method of any one of embodiments 308 to 316, wherein the subject has hepatic cirrhosis associated with the NAFLD.
319) The method of any one of embodiments 308 to 317, wherein the subject has hepatic cirrhosis as a comorbidity.
320) The method of any one of embodiments 308 to 318, wherein the subject has hepatic cirrhosis caused by the NAFLD.
321) The method of any one of embodiments 308 to 319, wherein the NAFLD is NAFL with attendant liver cirrhosis.
322) The method of any one of embodiments 308 to 320, wherein the treatment of the NAFLD comprises treatment of liver cirrhosis.
323) The method of any one of embodiments 308 to 321, wherein the treatment of NAFLD decreases the level of serum bile acids in the subject.
324) The method of any one of embodiments 308 to 322, wherein the treatment of NAFLD comprises treatment of pruritus.
325) The method of any one of embodiments 308 to 316, wherein the NAFLD is simple nonalcoholic fatty liver (NAFL).
326) The method of embodiment 324, wherein the treatment of NAFL comprises treatment of pruritus.
327) The method of embodiment 324, wherein the treatment of NAFL decreases the level of serum bile acids in the subject.
328) The method of any one of embodiments 308 to 319 or 321 to 323, wherein the NAFLD is nonalcoholic steatohepatitis (NASH).
329) The method of any one of embodiments 308 to 319, 321 to 323, or 327, wherein the NAFLD is NASH with attendant liver cirrhosis.
330) The method of any one of embodiments 308 to 319, 321 to 323, or 327 to 328, wherein the treatment of NASH decreases the level of serum bile acids in the subject.
331) The method of any one of embodiments 308 to 319, 321 to 323, or 327 to 329, wherein the treatment of NASH comprises treatment of pruritus.
332) A method of treating fibrosis in a subject in need thereof comprising administering to the subject

(c) the compound of Formula (I), or a pharmaceutically acceptable salt thereof, and

(d) a PCSK9 inhibitor,
wherein the amounts of (a) and (b) together are effective in treating NAFLD.
   333) The method of embodiment 331, wherein the compound of Formula I is

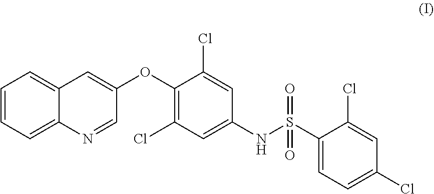 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
334) The method of embodiment 331 or 332, wherein the fibrosis is cirrhosis.
335) The method of any one of embodiments 331 to 333, wherein the fibrosis is associated with NAFLD.
336) The method of any one of embodiments 331 to 334, wherein the fibrosis is caused by NAFLD.
337) The method of any one of embodiments 331 to 335, wherein the NAFLD is NASH.
338) The method of any one of embodiments 331 to 336, wherein the treatment of fibrosis comprises a decrease in the stage of fibrosis, a lack of progression of the fibrosis, or a slowing in the progression of the fibrosis.
339) The method of any one of embodiments 331 to 337, wherein the treatment of fibrosis comprises a decrease in the stage of fibrosis.
340) The method of any one of embodiments 331 to 338, wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.
341) The method of any one of embodiments 308 to 339, wherein the PCSK9 inhibitor is selected from the group consisting of alirocumab, evolocumab, bococizumab, 1D05-IgG2, RG-7652, LY3015014, and inclisiran.
342) The method of any one of embodiments 308 to 340, wherein the PCSK9 inhibitor is alirocumab.
343) The method of any one of embodiments 308 to 341, wherein (a) and (b) are administered concurrently.
344) The method of any one of embodiments 308 to 341, wherein (a) and (b) are administered sequentially in either order.
345) The method of any one of embodiments 308 to 343, wherein the PCSK9 inhibitor is administered at a dose from about 50 to about 450 mg.
346) The method of any one of embodiments 308 to 344, wherein the PCSK9 inhibitor is administered at a dose from about 150 to about 200 mg.
347) The method of any one of embodiments 308 to 344, wherein the PCSK9 is administered at a dose from about 200 to about 250 mg.
348) The method of any one of embodiments 308 to 344, wherein the PCSK9 inhibitor is administered at a dose of about 250 to about 300 mg.
349) The method of any one of embodiments 308 to 344, wherein the PCSK9 inhibitor is administered at a dose of about 25 to about 50 mg.
350) The method of any one of embodiments 308 to 344, wherein the PCSK9 inhibitor is administered at a dose of about 40 to about 65 mg.
351) The method of any one of embodiments 308 to 344, wherein the PCSK9 inhibitor is administered at a dose of about 55 to about 80 mg.
352) The method of any one of embodiments 308 to 344, wherein the PCSK9 inhibitor is administered at a dose of about 70 to about 95 mg.
353) The method of any one of embodiments 308 to 347, wherein the PCSK9 inhibitor is administered to the subject monthly.
354) The method of any one of embodiments 308 to 343 or 348-352, wherein the PCSK9 inhibitor is administered to the subject twice a month.
355) The method of any one of embodiments 308 to 353, wherein the adiponectin level in the subject is increased by at least about 30%, at least about 68%, at least about 175%, or at least about 200%.
356) The method of any one of embodiments 308 to 354, wherein the level of one or more biomarkers indicative of one or more of liver damage, inflammation, fibrosis, and/or cirrhosis is decreased.
357) The method of embodiment 355, wherein the increase is by at least about 175%.
358) The method of any one of embodiments 308 to 356, wherein the compound of Formula (I), a pharmaceutically acceptable salt thereof, is administered prophylactically.
359) The method of any one of embodiments 308 to 357, wherein the compound of Formula (I), or a pharmaceutically acceptable salt thereof, is administered at a dose from about 0.1 to about 15 mg.
360) The method of any one of embodiments 308 to 358, wherein the compound of Formula (I), or a pharmaceutically acceptable salt thereof, is administered at a dose from about 1 to about 10 mg.
361) The method of any one of embodiments 308 to 359, wherein the compound of Formula (I), or a pharmaceutically acceptable salt thereof, is administered at a dose from about 2 to about 6 mg.
362) The method of any one of embodiments 308 to 358, wherein the compound of Formula (I), or a pharmaceutically acceptable salt thereof, is administered at a dose from about 0.5 to about 3 mg.
363) The method of any one of embodiments 308 to 361, wherein the compound of Formula (I), or a pharmaceutically acceptable salt thereof, is administered at a dose of about 3 mg.
364) The method of any one of embodiments 308 to 361, wherein the compound of Formula (I), or a pharmaceutically acceptable salt thereof, is administered at a dose of about 2 mg.
365) The method of any one of embodiments 308 to 359 or 361, wherein the compound of Formula (I), or a pharmaceutically acceptable salt thereof, is administered at a dose of about 1 mg.
366) The method of any one of embodiments 308 to 364, wherein the compound of Formula (I), or a pharmaceutically acceptable salt thereof, is administered to the subject daily, every other day, three times a week, twice a week, weekly, every other week, twice a month, or monthly.
367) The method of any one of embodiments 308 to 365, wherein the compound of Formula (I), or a pharmaceutically acceptable salt thereof, is administered to the subject daily.
368) The method of any one of embodiments 308 to 358, wherein the compound of Formula (I), or a pharmaceutically acceptable salt thereof, is administered to the subject daily and the dose of the compound of Formula (I) is about 3 mg.
369) The method of any one of embodiments 308 to 358, wherein the compound of Formula (I), or a pharmaceutically acceptable salt thereof, is administered at a dose from about 0.1 to about 10.0 mg per day.
370) The method of any one of embodiments 308 to 358 or 368, wherein the compound of Formula (I), or a pharmaceutically acceptable salt thereof, is administered at a dose from about 0.1 to about 3 mg per day.
371) The method of any one of embodiments 308 to 358 or 368 to 369, wherein the compound of Formula (I), or a pharmaceutically acceptable salt thereof, is administered at a dose of about 0.5 mg per day.
372) The method of any one of embodiments 308 to 358 or 368 to 369, wherein the compound of Formula (I), or a pharmaceutically acceptable salt thereof, is administered at a dose of about 1 mg per day.
373) The method of any one of embodiments 308 to 358 or 368 to 369, wherein the compound of Formula (I), or a pharmaceutically acceptable salt thereof, is administered at a dose of about 2 mg per day.
374) The method of any one of embodiments 308 to 372, wherein the method further comprises performing a liver biopsy to determine the NAFLD activity score of the biopsy sample obtained from the subject.
375) A method of treating non-alcoholic fatty liver disease (NAFLD) in a subject in need thereof consisting essentially of administering to the subject

(e) the compound of Formula (I), or a pharmaceutically acceptable salt thereof, and

(f) a PCSK9 inhibitor,
wherein the amounts of (a) and (b) together are effective in treating NAFLD.
   376) The method of any one of embodiments 308 to 374, wherein the compound of Formula (I) is in the form of a pharmaceutically acceptable salt.
   377) The method of any one of embodiments 308 to 375, wherein the compound of Formula (I) is in the form of a besylate salt.
   378) The method of any one of embodiments 308 to 374, wherein the compound of Formula (I) is in the form of a free base.
   379) A pharmaceutical combination comprising

(e) the compound of Formula (I), or a pharmaceutically acceptable salt thereof, and

(f) a PCSK9 inhibitor,

for concurrent or sequential administration for use in the treatment of non-alcoholic fatty liver disease (NAFLD).

380) The pharmaceutical combination of embodiment 378, wherein the compound of Formula (I) is in the form of a pharmaceutically acceptable salt.
381) The pharmaceutical combination of embodiment 378 or 379, further comprising at least one pharmaceutically acceptable carrier.

### EXAMPLES

The following examples further illustrate the invention. For example, the efficacy of CHS-131, alone or in combination with an additional agent such as an ASK1 inhibitor, an ACC inhibitor, a caspase inhibitor, a MCR antagonist, a statin, a PCSK9 inhibitor, or a pharmaceutically acceptable salt or solvate of any of the foregoing, to treat NAFLD is demonstrated in the following examples.

### General Procedures

The effects of treatment with CHS-131 (Compound of Formula (I)), alone and in combination with other therapeutic agents, to treat NASH are evaluated in mice. Various models can be used, such as the DIO-NASH model. Subjects are divided into groups for treatment and evaluation. Groups can include, controls (e.g. subjects on or off diets that are not administered a therapy), subjects administered monotherapy (e.g. CHS-131; an ASK1 inhibitor, an ACC inhibitor, a caspase inhibitor, a MCR antagonist, a statin, a PCSK9 inhibitor, or a pharmaceutically acceptable salt or solvate of any of the foregoing), subjects administered a combo-therapy (e.g. CHS-131 and compound selected from an ASK1 inhibitor, an ACC inhibitor, a caspase inhibitor, a MCR antagonist, a statin, a PCSK9 inhibitor, or a pharmaceutically acceptable salt or solvate of any of the foregoing), and subjects administered a positive control therapy. Metabolic parameters, hepatic pathology, and NAFLD Activity Score including fibrosis stage are evaluated.

Each animal is administered the respective compositions (e.g. vehicle, monotherapy, combo-therapy) starting on Day 0 and ending on Day 82-84. Samples, as described in Table 3, are collected for analysis.

TABLE 3
Samples collected over course of study

| Sample | Usage | Groups | Time Point | Method |
|---|---|---|---|---|
| Liver pre- | Stratification and | All | 3 weeks | Dissection |
| biopsy | randomization, | | before start | |
| | NAFLD Activity | | of study | |
| | Score, Fibrosis | | | |
| | Stage, Col1a1 | | | |
| BG Baseline | Blood Glucose | All | 1 week | Tail Vein |
| | before start | | | |
| | of study | | | |
| Plasma insulin | Plasma insulin | All | 1 week | Tail Vein |
| baseline | | | before start | |
| | | | of study | |
| OGTT | Blood Glucose | All | Week 7-8 | Tail Vein |
| IPTT | Blood Glucose | All | Week 9/10 | Tail Vein |
| BG week 12 | Blood Glucose | All | Week 12 | Tail Vein |
| Plasma insulin | Plasma insulin | All | Week 12 | Tail Vein |
| week 12 | | | | |
| Terminal | ALT/AST/TG/ | All | Week 12 | Tail Vein |
| ALT/AST/ | TC/BUN | | | |
| TG/TC/BUN/ | | | | |
| creatinine | | | | |
| Liver post- | NAFLD Activity | All | Termination | Dissection |
| biopsy | Score, and fibrosis | | | |
| | stage and steatosis | | | |
| | stage and Collal | | | |
| | and Galectin-3 | | | |
| | and a-SMA | | | |
| | quantification | | | |
| Liver TG/TC | Liver triglyceride | All | Termination | Dissection |
| | and total cholesterol | | | |
| Liver HP | Liver hydroxyproline | All | Termination | Dissection |
| Liver RNA | RNAseq (optional) | All | Termination | Dissection |
| Liver | Evaluation | All | Termination | Dissection |
| Muscle | Evaluation | All | Termination | Dissection |
| Epididymal fat | Evaluation | All | Termination | Dissection |
| Subcutaneous | Evaluation | All | Termination | Dissection |
| fat | | | | |
| Kidney | Evaluation | All | Termination | Dissection |
| Brain | Evaluation | All | Termination | Dissection |
| Heart | Evaluation | All | Termination | Dissection |
| Terminal | Evaluation | All | Termination | Cardiac |
| plasma | | | | Puncture |

ALT is alanine transaminase;
a-SMA is alpha-smooth muscle actin;
AST is aspartate transaminase;
BG is blood glucose;
BUN is blood urea nitrogen;
Col1a1 is collagen 1a1;
OGTT is oral glucose tolerance test;
IPITT is intraperitoneal insulin tolerance test;
TG is triglycerides;
TC is total cholesterol;
HP is hydroxyproline

An overview of sample analyses that are performed during the study are listed in Tables 4-6, below.

TABLE 4
In vivo pharmacology

| Analysis | | | Period or | |
|---|---|---|---|---|
| Name | Groups | Samples | Frequency | Comments |
| Bodyweight | All | Whole | QD | na |
| | | animal | | |
| Food intake | All | Whole | QD week 0 + 1 | AM |
| | | animal | QW (24 h) | |
| | | | Week 2-12 | |
| Echo MRI | All | Whole | Week 1 | na |
| baseline | | animal | | |
| Echo MRI | All | Whole | Week 11 | na |
| week 11 | | animal | | |
| Liver | All | Whole liver | Termination | na |
| weight | | weight (wet | | |
| | | weight) | | |

TABLE 5
Histology

| Analysis Name | Groups | Samples | Comments |
|---|---|---|---|
| Fibrosis stage | All | Liver pre-biopsy | PSR staining |
| | | Liver post-biopsy | Re-staining of pre-biopsy |
| NAFLD | All | Liver pre-biopsy | HE staining |
| Activity | | Liver post-biopsy | Re-staining of pre-biopsy |
| Score | | | |
| Col1a1 | All | Liver pre-biopsy | IHC (randomization) |
| | | Liver post-biopsy | IHC |
| Galectin-3 | All | Liver post-biopsy | IHC |
| quantification | | | |
| Steatosis | All | Liver post-biopsy | HE staining |
| quantification | | | |
| a-SMA | All | Liver post-biopsy | IHC |
| quantification | | | |

TABLE 6
Assays

| Analysis Name | Groups | Samples |
|---|---|---|
| Plasma insulin | All | Plasma insulin baseline |
| baseline | | |
| Plasma insulin | All | Plasma insulin baseline |
| week 12 | | |
| Plasma ALT | All | Terminal ALT/AST/TG/TC/BUN/Creatinine |
| Plasma AST | All | Terminal ALT/AST/TG/TC/BUN/Creatinine |
| Plasma TG | All | Terminal ALT/AST/TG/TC/BUN/Creatinine |
| Plasma TC | All | Terminal ALT/AST/TG/TC/BUN/Creatinine |
| Plasma BUN | All | Terminal ALT/AST/TG/TC/BUN/Creatinine |
| Plasma creatinine | All | Terminal ALT/AST/TG/TC/BUN/Creatinine |
| Liver triglycerides | All | Liver TG/TC |
| Liver total | All | Liver TG/TC |
| cholesterol | | |
| Liver | All | Liver HP |
| hydroxyproline | | |

NAFLD Activity Score (NAS) and Fibrosis stage are evaluated as follows. Liver samples are fixed in formalin, paraffin embedded and sections are stained with hematoxylin and eosin (H&E) and Sirius Red. Samples are scored for NAS and fibrosis stage (outlined below) using of the clinical criteria outlined by Kleiner et al. 2005. Total NAS score represents the sum of scores for steatosis, inflammation, and ballooning, and ranges from 0-8.

TABLE 7
Total NAS scoring

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

Adopted from: Design and validation of a histological scoring system for non-alcoholic fatty liver disease, Kleiner et al., Hepatology 41; 2005.

For lobular inflammation, inflammation is evaluated by counting the number of inflammatory foci per field using a 200× magnification (min. 5 fields per animal). A focus is defined as a cluster, not a row, of >3 inflammatory cells. Acidophil bodies are not included in this assessment, nor is portal inflammation. Fibrosis stage is evaluated separately from NAS.

### IHC and Steatosis Quantification

Quantitative assessment of immunoreactivity is evaluated as follows. IHC-positive staining is quantified by image analysis using the Visiomorph software (Visiopharm, Denmark). Visiomorph protocols are designed to analyze the virtual slides in two steps: 1. Crude detection of tissue at low magnification (1× objective). The liver capsule is excluded. 2. Detection of IHC-positive staining (e.g., green; collagen 1 IHC), tissue (e.g., red) and fat (e.g., pink) at high magnification (10× objective). The quantitative estimate of IHC-positive staining is calculated as an area fraction (AF) according to the following formula:

$$A {F}_{IHC \text{-} pos} = \frac{A r e {a}_{IHC \text{-} pos .}}{A r e {a}_{fat} + A r e {a}_{tissue} + A r e {a}_{IHC \text{-} pos}}$$

Quantitative assessment of steatosis is evaluated as follows. Steatosis is quantified on H&E stained slides by image analysis using the Visiomorph software (Visiopharm, Denmark). Visiomorph protocols are designed to analyse the virtual slides in two steps: 1. Crude detection of tissue at low magnification (1× objective). 2. Detection of steatosis (pink) and tissue (blue) at high magnification (20× objective). The quantitative estimate of steatosis is calculated as an area fraction (AF) according to the following formula:

$$A {F}_{steatosis} = \frac{A r e {a}_{steatosis}}{A r e {a}_{tissue} + A r e {a}_{steatosis}}$$

### Example 2

This study assesses the effects of treatment with CHS-131 (Compound of Formula (I)), alone and in combination with other therapeutic agents, to treat NASH. Metabolic parameters, hepatic pathology, and NAFLD Activity Score including fibrosis stage are evaluated in male DIO-NASH mice. The other therapeutic agents are: (i) an ASK1 inhibitor, or a pharmaceutically acceptable salt thereof (e.g., selonsertib); (ii) an ACC inhibitor, or a pharmaceutically acceptable salt thereof; (iii) a caspase inhibitor, or a pharmaceutically acceptable salt thereof (e.g., emricasan); (iv) a MCR antagonist, or a pharmaceutically acceptable salt thereof; (v) a statin, or a pharmaceutically acceptable salt thereof (e.g., atorvastatin); or (vi) a PCSK9 inhibitor, or a pharmaceutically acceptable salt thereof (e.g., alirocumab, evolocumab, bococizumab, or inclisiran).

Abbreviations used herein include: Alanine aminotransferase (ALT), Amylin liver NASH (AMLN), Aspartate aminotransferase (AST), Body weight (BW), Carboxy Methylcellulose CMC( ) Collagen 1A1 (Col1a1), Diet Induced obesity (DIO), Galectin-3 (Gal-3), Hematoxylin & Eosin (HE), Immunohistochemistry (IHC), Hydroxyproline (HP), Nonalcoholic fatty liver disease (NAFLD), NAFLD Activity Score (NAS), Nonalcoholic steatohepatitis (NASH), Per oral (PO), Total cholesterol (TC), Triglycerides (TG), Alpha-smooth muscle actin (α-SMA).

### Materials and Methods

### Mouse Model, NASH Induction and Randomization

### Mouse Strain

The animals used are male C57BL/6JRj mice supplied by JanVier (France) at 5 weeks of age.

### NASH Induction

The Diet-induced-obesity (DIO)-NASH mouse model is induced by feeding male C57BL/6JRj mice a high fat diet containing 40% fat with trans-fat, 20% fructose and 2% cholesterol (AMLN diet or D09100301, Research Diets Inc., USA). Induction of NASH is started at 5 weeks of age and mice are fed the AMLN diet for 36 weeks prior to study start resulting in NASH, which is confirmed by pre-biopsy prior to study start as described below.

### PRE-Biopsy Procedure and Randomization

Three weeks prior to study start, a pre-biopsy is performed to confirm NASH and for study inclusion of NASH-affected mice only. Briefly, mice are anesthetized with isoflurane (2-3%) in 100% oxygen. A small abdominal incision is made in the midline and the left lateral lobe of the liver exposed. A cone shaped wedge of liver tissue (approximately 50 mg) is excised from the distal portion of the lobe and fixated in 10% neutral buffered formalin (4% formaldehyde) for histopathological analyses. The cut surface of the liver is instantly electro-coagulated using bipolar coagulation (ERBE VIO 100 electrosurgical unit). The liver is returned to the abdominal cavity, the abdominal wall sutured, and the skin closed with staplers. For post-operative recovery, mice received carprofen (5 mg/mL-0.01 mL/10 g) administered subcutaneously on the day of operation and on post-operation day 1 and 2.

After surgery, the animals are evaluated daily on general health and body weight. In previous evaluations, an animal having sham surgery (just the abdominal incision) had the same body weight loss as an animal with a liver biopsy; around 10%. No evidence of greater pain (visceral pain) are observed in the animals where a biopsy is taken, compared to sham-operated animals. Signs of concerning pain or suffering has not been observed previously, and no animals had to be terminated (internal observations). The pre-biopsy is analyzed to evaluate liver steatosis score and fibrosis stage for study inclusion as outlined by Kleiner et al. (2005) (Table 1). In addition, liver Collagen 1a1 (Col1a1) quantified by morphometry is used to perform a stratified randomization of NASH-affected animals into study groups (see description of histopathological stains and analyses below).

### Formulation of Compounds

### Test Substances

CHS-131 and the other therapeutic agents are prepared appropriately for dosing (e.g., CHS-131 is suspended in 1% Methyl cellulose (MC) in deionized water). Dosages are prepared weekly and protected from light.

### Route and Dose of Drug Administration

CHS-131 is administered at a dose of 10 mg/kg (low) or 30 mg/kg (high) once a day (AM).

All compounds are administered at dose volume of 5 mL/kg via oral gavage (passed through the mouth into the stomach where the dosage is deposited) or subcutaneous or intraperitoneal injection. The suspensions are stirred for 60 minutes before and during dosing.

### Tolerance Tests

### Intraperitoneal Insulin Tolerance Test

Mice are fasted 6 hours prior to intraperitoneal insulin administration (0.5 Unit/kg, rapid acting insulin NovoRapid). At the various time points after insulin administration, blood samples are collected into heparinized glass capillary tubes and immediately suspended in glucose/lactate system solution buffer (EKF-diagnostics, Germany). Blood glucose (BG) is measured using a BIOSEN c-Line glucose meter (EKF-diagnostics, Germany) according to the manufacturer's instructions. After the last blood sample, the animals are returned to the normal feeding schedule. The order of the animals is randomized before the procedure and mice are dosed with compounds just after the −60 minutes blood sample.

### Oral Glucose Tolerance Test

Animals are fasted 6 hours prior to oral glucose administration (2 g/kg). At the various time points after glucose administration, blood samples are collected into heparinized glass capillary tubes and immediately suspended in glucose/lactate system solution buffer (EKF-diagnostics, Germany). Blood glucose (BG) is measured using a BIOSEN c-Line glucose meter (EKF-diagnostics, Germany) according to the manufacturer's instructions. After the last blood sample, the animals are returned to the normal feeding schedule. The order of the animals are randomized before the procedure and mice are dosed with compounds just after the −60 minutes blood sample.

### EchoMRI Body Composition

The body composition of the mice is assessed by an EchoMRI 3-1 Body composition analyzer (EchoMRI, US). Non-anaesthetised mice is placed in a plastic tube inside the MRI scanner for approximately 80 seconds. The body composition is expressed as fat mass, fat free mass (lean mass) and water.

### Termination and Sample Collection

### Blood Sampling and Plasma Preparation

For plasma biochemistry, tail blood is drawn directly through the capillary of a Microvette/Vacuette of the right dimension and anticoagulant and mixed by inversion 5 times. Blood is placed at 4° C. until centrifugation at 3000×g for 10 minutes at 4° C. The plasma supernatants are transferred to new tubes and immediately frozen on dry ice and stored at −80° C. until analysis.

### Termination

Animals are terminated after 12 weeks of treatment in a non-fasting state. Animals are put under isoflurane anesthesia, the abdominal cavity is opened, and cardiac blood is drawn directly into a Vacuette of the right dimension and anticoagulant and mixed by inversion 5 times. Blood is placed at 4° C. until centrifugation at 3000×g for 10 minutes at 4° C. The plasma supernatants are transferred to new tubes and immediately frozen on dry ice and stored at −80° C. Upon necropsy, the whole liver is collected and weighed. The liver is sampled for histological and biochemical analyses as described below.

### Liver Sampling and Sample Preparation

The liver post-biopsy for histological analyses is removed by dissection from the left lateral lobe, fixated in 4% formalin for 20-24 h, and subsequently embedded in paraffin. Liver biopsies for liver triglycerides and total cholesterol are dissected from the medial lobe, snap frozen in liquid nitrogen, and stored at −80° C., while liver biopsies for hydroxyproline are dissected from the caudal lobe (the entire lobe), snap frozen in liquid nitrogen and stored at −80° C. Finally, a liver sample for RNA isolation and gene expression analysis is dissected from the left lateral lobe, snap frozen in liquid nitrogen, and stored at −80° C. until processing.

### Measurement of Plasma and Liver Biochemistry

### Measurement of Plasma Biochemistry

Plasma alanine transaminase (ALT) (Roche Diagnostics), aspartate transaminase (AST) (Roche Diagnostics), triglycerides (TG) (Roche Diagnostics), total cholesterol (TC) (Roche Diagnostics), creatinine (Roche Diagnostics), and urea (Roche Diagnostics) are measured using commercial kits on the Cobas c 501 autoanalyzer according to the manufacturer's instructions. Mouse insulin is measured in single determinations using the MSD platform (Meso Scale Diagnostics).

### Measurement of Liver Biochemistry

For liver hydroxyproline (HP; a protein marker of fibrosis) quantification, liver samples are homogenized in 6 M HCl and hydrolyzed to degrade collagen. The samples are centrifuged, and the hydroxyproline content measured in duplicates in the supernatant, using a colorimetric assay (Quickzyme Biosciences) according to the manufacturer's instructions.

For liver TG and TC quantification, samples are homogenized, and TG and TC extracted in 5% NP-40 by heating twice to 90° C. The samples are centrifuged, and the TG and TC content measured in the supernatant, using commercial kits (Roche Diagnostics) on the Cobas c501 autoanalyzer according to the manufacturer's instructions.

### Histological Tissue Preparation and Staining Procedures

### Histological Tissue Preparation

Liver biopsies fixated in formalin are infiltrated over-night in paraffin in an automated Miles Scientific Tissue-TEK VIP Tissue Processor and subsequently embedded in paraffin blocks, which are trimmed and from which 3 μm thick sections are cut on a Microm HM340E Microtome. Slides with paraffin-embedded sections are de-paraffinated in xylene and rehydrated in a series of graded ethanol prior to histochemical or immunohistochemical (IHC) staining.

### Histochemical Stains

For Hematoxylin & Eosin (HE) staining, slides are incubated in Mayer's Hematoxylin, washed in tap water, stained in Eosin Y solution, hydrated, mounted with Pertex and allowed to dry before scanning.

For Sirius red staining, slides are incubated in Weigert's iron hematoxylin, washed in tap water, stained in Picro-Sirius red and washed twice in acidified water. Excess water is removed by shaking the slides after which the slides are dehydrated in three changes of 100% ethanol, cleared in xylene, mounted with Pertex and allowed to dry before scanning.

### Immunohistochemical Stains

Protein markers of fibrosis (Col1a1), fibrogenesis (α-SMA) and inflammation (Gal-3) are assessed by immunohistochemistry. α-SMA and collagen type I increase in regulation of quiescent hepatic stellate cell activation into myofibroblast-like cells where activated hepatic stellate cells are the main collagen producing cells in the liver (Carpino et al 2005, Hou and Syn 2018) whereas Gal-3 is involved in mediating inflammatory response and considered as a macrophage activation marker (Sciacchitano et al, 2018). For morphometric quantification of liver Col1a1 (using antibody from Southern Biotech, Cat. #1310-01), alpha-smooth muscle actin (α-SMA; using antibody from Abcam, Cat. #Ab124964) and Galectin-3 (using antibody from Biolegend, Cat. #125402), IHC staining is performed using standard procedures. Briefly, after antigen retrieval and blocking of endogenous peroxidase activity, slides are incubated with primary antibody. For all IHC stains, the primary antibody is detected using a polymeric HRP-linker antibody conjugate and visualized using DAB as chromogen. Finally, sections are counterstained in hematoxylin and cover-slipped before scanning.

### NAFLD Activity Score and Fibrosis Stage

For scoring of NAFLD Activity Score (NAS) and fibrosis stage, HE and Sirius red stained liver sections, respectively, are scored by a histopathology specialist as outlined in Table 10 using the clinical criteria outlined by Kleiner et al. (2005). Total NAS score represents the sum of scores for steatosis, lobular inflammation, and ballooning degeneration scores, and ranges from 0-8.

TABLE 10

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

For steatosis score, percentage refers to percentage of hepatocytes affected by steatosis as evaluated on low to medium power examination.

For lobular inflammation, inflammation is evaluated by counting the number of inflammatory foci per field using a 200× magnification (min. 5 fields per animal). A focus is defined as a cluster, not a row, of >3 inflammatory cells. Acidophil bodies are not included in this assessment, nor is portal inflammation.

For hepatocellular ballooning degeneration, degenerated hepatocytes with a cleared cytoplasm, enlargement, swelling, rounding and reticulated cytoplasm are identified.

Fibrosis stage is evaluated separately from NAS.

### IHC and Steatosis Quantification

Quantitative assessment of immunoreactivity is evaluated as follows. IHC-positive staining is quantified by image analysis using the Visiomorph software (Visiopharm, Denmark). Visiomorph protocols are designed to analyze the virtual slides in two steps: 1. Crude detection of tissue at low magnification (1× objective). The liver capsule is excluded. 2. Detection of IHC-positive staining (e.g. green; collagen 1 IHC), tissue (e.g. red) and fat (e.g. pink) at high magnification (10× objective). The quantitative estimate of IHC-positive staining is calculated as an area fraction (AF) according to the following formula:

$$A {F}_{IHC \text{-} pos} = \frac{A r e {a}_{IHC \text{-} pos .}}{A r e {a}_{fat} + A r e {a}_{tissue} + A r e {a}_{IHC \text{-} pos}}$$

Quantitative assessment of steatosis is evaluated as follows. Steatosis is quantified on H&E stained slides by image analysis using the Visiomorph software (Visiopharm, Denmark). Visiomorph protocols are designed to analyze the virtual slides in two steps: 1. Crude detection of tissue at low magnification (1× objective). 2. Detection of steatosis (pink) and tissue (blue) at high magnification (20× objective). The quantitative estimate of steatosis is calculated as an area fraction (AF) according to the following formula:

$$A {F}_{steatosis} = \frac{A r e {a}_{steatosis}}{A r e {a}_{tissue} + A r e {a}_{steatosis}}$$

### Statistical Tests

For single-timepoint continuous data, the data are fitted to a one-factor linear regression model with the treatment groups as categorical, independent (predictor) variables and Dunnett's test is used to compare treatments to the Vehicle control.

Data regarding liver fibrosis, absolute body weight, relative body weight, MRI body weight, daily food intake, cumulative food intake, absolute fat tissue mass, relative fat tissue mass, absolute lean tissue mass, relative lean tissue mass, absolute free water mass, relative free water mass, fasted blood glucose, fasted plasma insulin, glucose tolerance as assessed by oral glucose tolerance test, insulin sensitivity as assessed by intraperitoneal insulin tolerance test, terminal plasma total cholesterol, terminal plasma ALT and AST, plasma urea at termination, absolute liver weight, relative liver weight, relative and total liver total cholesterol at termination, relative and total terminal liver triglycerides, relative liver hydroxyproline levels at termination, change in NAFLD activity score, relative and total liver steatosis, relative and total liver Col1a1 content, relative and total liver α-SMA levels at termination, and relative and total liver Galectin-3 levels at termination are collected for the following treatment groups:

Treatment
CHOW vehicle + vehicle
NASH vehicle + vehicle
CHS-131 Low + Vehicle
CHS-131 High + Vehicle
Vehicle + an ASK1 inhibitor as described herein
Vehicle + an ACC inhibitor as described herein
Vehicle + a caspase inhibitor, as described herein
Vehicle + a MCR antagonist, as described herein
Vehicle + a statin, as described herein
Vehicle + a PCSK9 inhibitor, as described herein
CHS-131 High + an ASK1 inhibitor as described herein
CHS-131 High + an ACC inhibitor as described herein
CHS-131 High + a caspase inhibitor, as described herein
CHS-131 High + a MCR antagonist, as described herein
CHS-131 High + a statin, as described herein
CHS-131 High + a PCSK9 inhibitor, as described herein

### Example 3

This study assesses the effects of treatment with CHS-131 (Compound of Formula (I)), alone and in combination with other therapeutic agents, to treat NASH. The other therapeutic agents are: (i) an ASK1 inhibitor, or a pharmaceutically acceptable salt thereof (e.g., selonsertib); (ii) an ACC inhibitor, or a pharmaceutically acceptable salt thereof; (iii) a caspase inhibitor, or a pharmaceutically acceptable salt thereof (e.g., emricasan); (iv) a MCR antagonist, or a pharmaceutically acceptable salt thereof; (v) a statin, or a pharmaceutically acceptable salt thereof (e.g., atorvastatin); or (vi) a PCSK9 inhibitor, or a pharmaceutically acceptable salt thereof (e.g., alirocumab, evolocumab, bococizumab, or inclisiran).

Metabolic parameters, hepatic pathology, and NAFLD Activity Score including fibrosis stage are evaluated in ob/ob mice. In addition to the description below, this study may include sample collection, testing, measurement, and evaluation (e.g. histology, biochemical, gene expression, genetic), and analysis as described in the examples above.

ob/ob mice are homozygous for a spontaneous Lep^{th} point mutation in the gene encoding leptin and are consistently fibrosis prone when cholesterol (2%) and trans-fatty acids (45% of total fat amount) are added to a high-caloric diet. These mice will develop steatohepatitis and fibrosis within a shorter timeframe (≤12 weeks) compared with wild-type C57BL/6 mice fed the same diet (≥26 weeks). See, e.g., Kristiansen, et al., World J. Hepatol., Vol. 8, pp. 673-684 (2016). The ob/ob mice also display a more significant insulin resistant and NASH phenotype than the high-caloric diet, well suited for evaluating potential anti-NASH therapeutics. Protocols for evaluating treatment of NASH in mouse models are found in Tølbøl, et al., World J Gastroenterol. 2018 Jan. 14; 24(2):179-194, Roth, et al., Sci Rep. 2019 Jun. 21; 9(1):9046, and Boland, et al., World J Gastroenterol. 2019 Sep. 7; 25(33):4904-4920, which are hereby incorporated by reference in their entirety.

In this study, ob/ob-NASH mice are divided into 4 ob/ob-NASH groups (e.g. n=14 for each group) with dosing for 12 weeks (PO, QD). Male B6.V-Lep^{ob}/JRj mice are fed 40% HFD, 20% fructose, 2% Cholesterol (GAN) diet for 12+ weeks prior to study start.

All mice entering the experiment are pre-biopsied at week −4 and stratified based on liver biopsy with only animals with fibrosis stage ≥1, inflammation score ≥2 and steatosis score ≥2 being included in the study. Animals are randomized into groups based on fibrosis stage as measured by picosirius red (PSR) staining. Total of 12 weeks of PO, QD dosing. The four groups are as follows: 1) Vehicle; 2) CHS-131, 30 mg/kg; 3) an ASK1 inhibitor, or a pharmaceutically acceptable salt thereof (e.g., selonsertib); 4) an ACC, or a pharmaceutically acceptable salt thereof; 5) a caspase inhibitor, or a pharmaceutically acceptable salt thereof (e.g., emricasan); 6) a MCR antagonist, or a pharmaceutically acceptable salt thereof; 7) a statin, or a pharmaceutically acceptable salt thereof (e.g., atorvastatin); 8) a PCSK9 inhibitor, or a pharmaceutically acceptable salt thereof (e.g., alirocumab, evolocumab, bococizumab, or inclisiran); 9) CHS-131, 30 mg/kg+an ASK1 inhibitor, or a pharmaceutically acceptable salt thereof (e.g., selonsertib); 10) CHS-131, 30 mg/kg+an ACC, or a pharmaceutically acceptable salt thereof; 11) CHS-131, 30 mg/kg+a caspase inhibitor, or a pharmaceutically acceptable salt thereof (e.g., emricasan); 12) CHS-131, 30 mg/kg+a MCR antagonist, or a pharmaceutically acceptable salt thereof; 13) CHS-131, 30 mg/kg+a statin, or a pharmaceutically acceptable salt thereof (e.g., atorvastatin); or 14) CHS-131, 30 mg/kg+a PCSK9 inhibitor, or a pharmaceutically acceptable salt thereof (e.g., alirocumab, evolocumab, bococizumab, or inclisiran).

Body weight is measured daily during the study period. Four hour fasting plasma glucose and HbA1c are measured at baseline, week 6, and week 12. Fasting plasma insulin and terminal plasma ALT/AST/GGT/and lipids are also measured at baseline and at week 12.

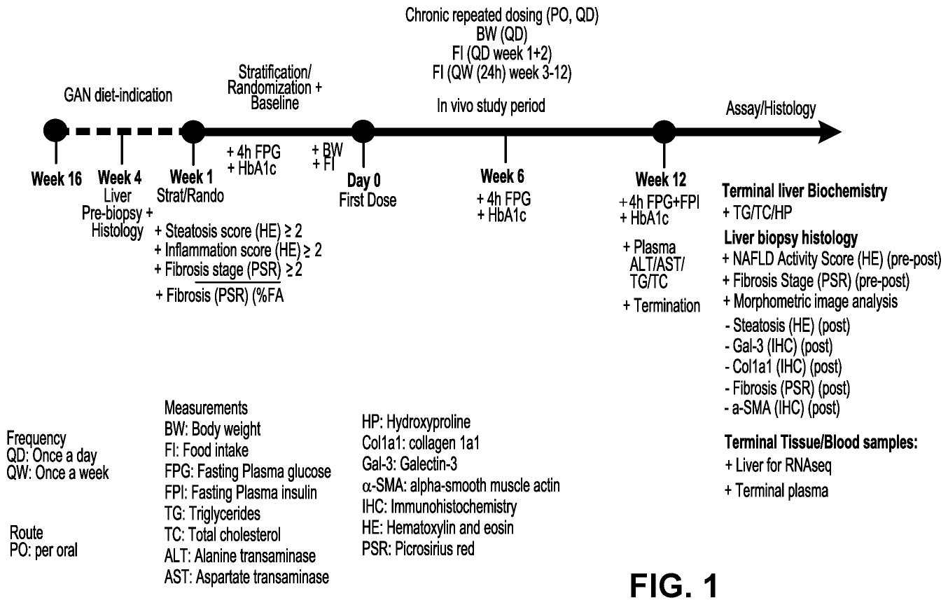

Terminal liver removal, weighing, and sampling at week 12 includes 1) FFPE (histology), 2) biochemical analysis, and 3) RNAseq analysis. Liver biopsy histology includes determination of 1) pre-to-post NAFLD Activity Score including Fibrosis Stage, 2) post steatosis (HE), 3) post Galectin-3 (IHC), an inflammation biomarker; other marker of an inflammatory response such as eicosanoids, hydroxyeicosatetraenoic acids (HETEs) and prostaglandins, are also measured, 4) post-fibrosis (PSR), 5) fibrosis biomarkers, including post Col1a1 (IHC), 6) post α-SMA (IHC). Additional fibrosis biomarkers are optionally measured including Pro-C3, C3M, Pro-C6 and C6M (Nordic Biosciences, Herlev, Denmark) which may characterize an observed anti-fibrotic effect. Liver TG/TC/HP content is also determined. Total adiponectin is measured at baseline and end-of-study. A study outline is shown in FIG. 1.

1-75. (canceled)

76. A method of treating non-alcoholic fatty liver disease (NAFLD) in a subject in need thereof comprising administering to the subject
(a) the compound of Formula (I), 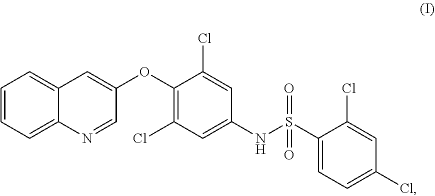 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a compound selected from the group consisting of an ASK1 inhibitor, an ACC inhibitor, a caspase inhibitor, a MCR antagonist, a statin, a PCSK9 inhibitor, or a pharmaceutically acceptable salt or solvate of any of the foregoing,
wherein the amounts of (a) and (b) together are effective in treating NAFLD.

77. The method of claim 76, wherein the NAFLD is nonalcoholic steatohepatitis (NASH).

78. The method of claim 76, wherein the NAFLD is NASH with attendant liver cirrhosis.

79. The method of claim 77, wherein the treatment of NASH decreases the level of serum bile acids in the subject.

80. The method of claim 77, wherein the treatment of NASH comprises treatment of pruritus.

81. The method of claim 76, wherein the NAFLD activity score (NAS) following administration is 7 or less; or is 5 or less; or is 3 or less.

82. The method of claim 76, wherein the compound is an ASK1 inhibitor selected from the group consisting of: selonsertib, MSC 2032964A, NQDI 1, TC ASK 10, AGI-1067, GS-444217, K811, K812, and SRT-015.

83. The method of claim 76, wherein the compound is an ACC inhibitor selected from the group consisting of:

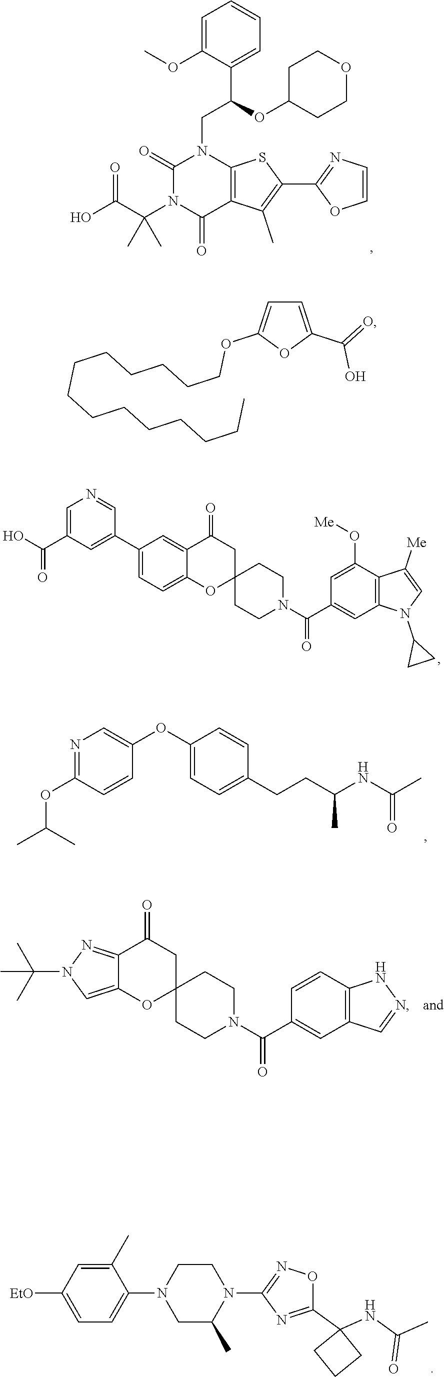

84. The method of claim 76, wherein the compound is a caspase inhibitor selected from the group consisting of: emricasan,

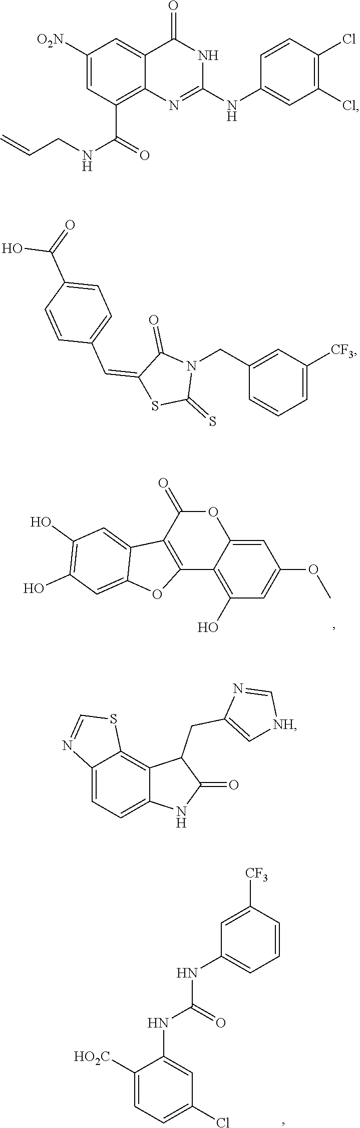 [SMILES: C=CCNC(=O)c1cc([N+](=O)[O-])cc2c(=O)[nH]c(Nc3ccc(Cl)c(Cl)c3)nc12 ; O=C(O)c1ccc(/C=C2/SC(=S)N(Cc3cccc(C(F)(F)F)c3)C2=O)cc1 ; COc1cc(O)c2c(c1)oc(=O)c1c3cc(O)c(O)cc3oc21 ; O=C1Nc2ccc3ncsc3c2C1Cc1c[nH]cn1 ; O=C(Nc1cccc(C(F)(F)F)c1)Nc1cc(Cl)ccc1C(=O)O]

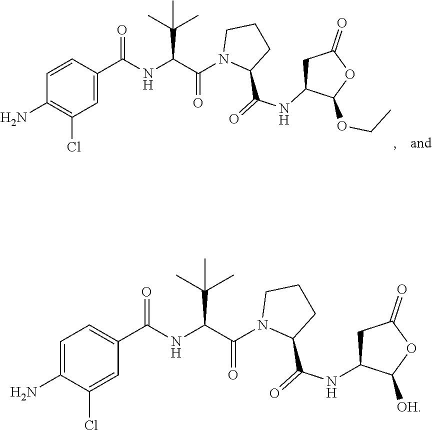 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C ; CC(C)(C)[C@H](NC(=O)c1ccc(N)c(Cl)c1)C(=O)N1CCC[C@H]1C(=O)N[C@H]1CC(=O)O[C@H]1O]

85. The method of claim 76, wherein the compound is a MCR antagonist selected from the group consisting of: spironolactone, finerenone, eplerenone, canrenone, mexrenone, amlodipine, apararenone, benidipine, esaxerenone, felodipine, nefedipine, nimodipine, and nitrendipine.

86. The method of claim 76, wherein the compound is a statin selected from the group consisting of: atorvastatin, cerivastatin, fluvastatin, lovastatin, mevastatin, pitavastatin, pravastatin, rosuvastatin, and simvastatin.

87. The method of claim 76, wherein the compound is a PCSK9 inhibitor selected from the group consisting of: alirocumab, evolocumab, bococizumab, 1D05-IgG2, RG-7652, LY3015014, and inclisiran.

88. A method of treating fibrosis in a subject in need thereof comprising administering to the subject
(a) the compound of Formula (I), 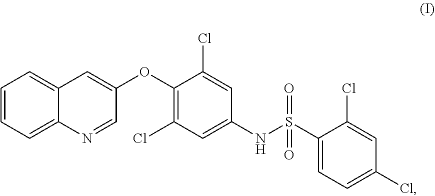 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof, and
(b) a compound selected from the group consisting of an ASK1 inhibitor, an ACC inhibitor, a caspase inhibitor, a MCR antagonist, a statin, a PCSK9 inhibitor, or a pharmaceutically acceptable salt or solvate of any of the foregoing,
wherein the amounts of (a) and (b) together are effective in treating NAFLD.

89. The method of claim 88, wherein the compound is an ASK1 inhibitor selected from the group consisting of: selonsertib, MSC 2032964A, NQDI 1, TC ASK 10, AGI-1067, GS-444217, K811, K812, and SRT-015.

90. The method of claim 88, wherein the compound is an ACC inhibitor selected from the group consisting of:

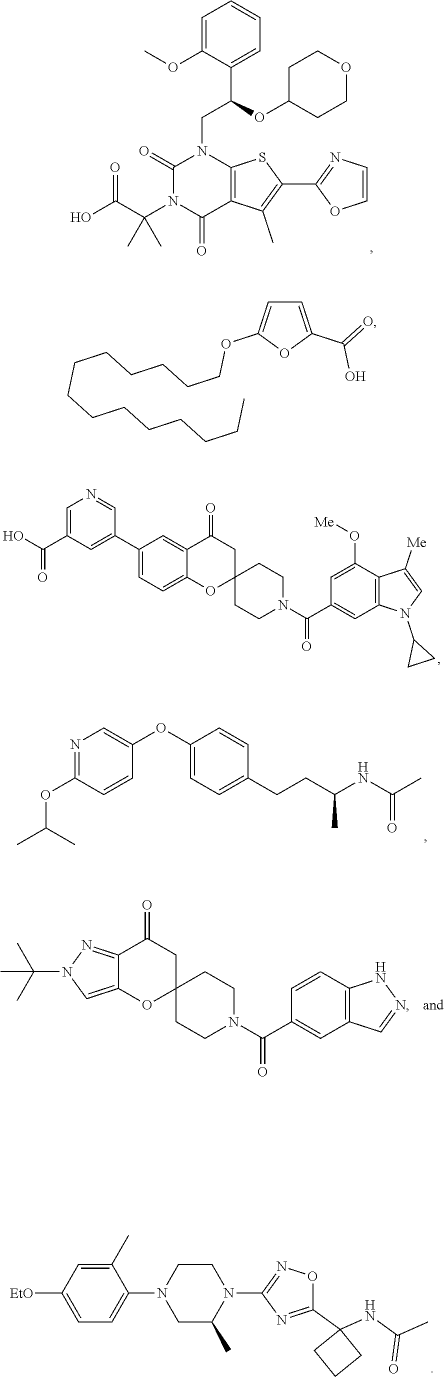

91. The method of claim 88, wherein the compound is a caspase inhibitor selected from the group consisting of: emricasan,

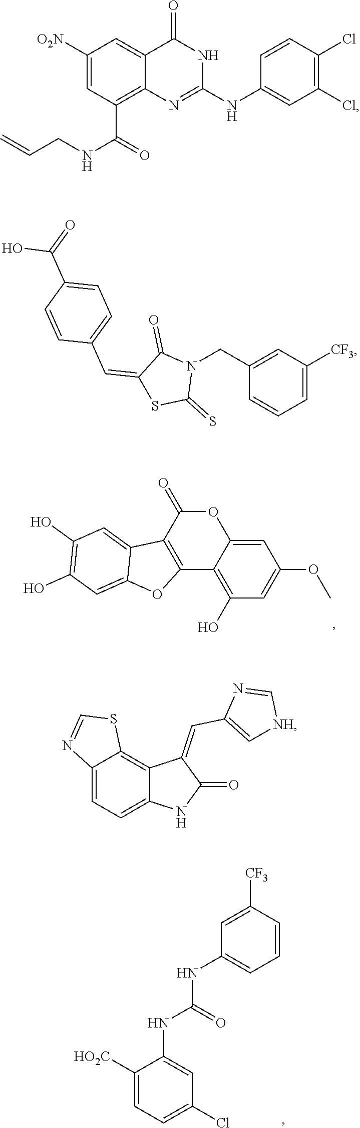 [SMILES: C=CCNC(=O)c1cc([N+](=O)[O-])cc2c(=O)[nH]c(Nc3ccc(Cl)c(Cl)c3)nc12 ; O=C(O)c1ccc(/C=C2/SC(=S)N(Cc3cccc(C(F)(F)F)c3)C2=O)cc1 ; COc1cc(O)c2c(c1)oc(=O)c1c3cc(O)c(O)cc3oc21 ; O=C1Nc2ccc3ncsc3c2/C1=C/c1c[nH]cn1 ; O=C(Nc1cccc(C(F)(F)F)c1)Nc1cc(Cl)ccc1C(=O)O]

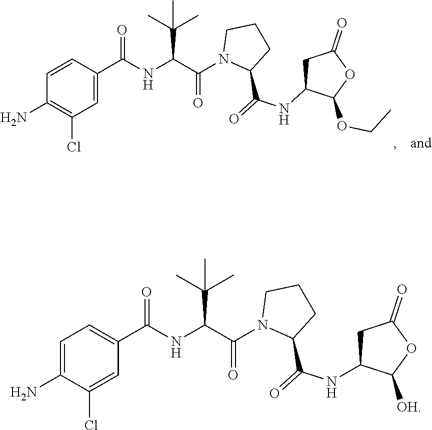 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C ; CC(C)(C)[C@H](NC(=O)c1ccc(N)c(Cl)c1)C(=O)N1CCC[C@H]1C(=O)N[C@H]1CC(=O)O[C@H]1O]

92. The method of claim 88, wherein the compound is a MCR antagonist selected from the group consisting of: spironolactone, finerenone, eplerenone, canrenone, mexrenone, amlodipine, apararenone, benidipine, esaxerenone, felodipine, nefedipine, nimodipine, and nitrendipine.

93. The method of claim 88, wherein the compound is a statin selected from the group consisting of: atorvastatin, cerivastatin, fluvastatin, lovastatin, mevastatin, pitavastatin, pravastatin, rosuvastatin, and simvastatin.

94. The method of claim 88, wherein the compound is a PCSK9 inhibitor selected from the group consisting of: alirocumab, evolocumab, bococizumab, 1D05-IgG2, RG-7652, LY3015014, and inclisiran.

95. A pharmaceutical composition comprising
(a) the compound of Formula (I), 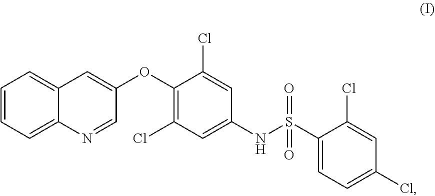 [SMILES: O=S(=O)(Nc1cc(Cl)c(Oc2cnc3ccccc3c2)c(Cl)c1)c1ccc(Cl)cc1Cl]
or a pharmaceutically acceptable salt or solvate thereof,
(b) a compound selected from the group consisting of an ASK1 inhibitor, an ACC inhibitor, a caspase inhibitor, a MCR antagonist, a statin, a PCSK9 inhibitor, or a pharmaceutically acceptable salt or solvate of any of the foregoing, and
one or more pharmaceutical excipients.
